(19) United States
(12) Patent Application Publication
(10) Pub. No.: US 2018/0263539 A1
(43) Pub. Date: Sep. 20, 2018

(54) WEARABLE SENSOR ARRAYS FOR IN-SITU BODY FLUID ANALYSIS

(71) Applicants: The Regents of the University of California, Oakland, CA (US); The Board of Trustees of the Stanford Junior University, Stanford, CA (US)

(72) Inventors: Ali Javey, Lafayette, CA (US); Wei Gao, San Jose, CA (US); Ronald W. Davis, Palo Alto, CA (US); Sam Emaminejad, Emeryville, CA (US)

(21) Appl. No.: 15/758,327

(22) PCT Filed: Sep. 27, 2016
(86) PCT No.: PCT/US16/53988

§ 371 Date: Mar. 7, 2018

Related U.S. Application Data
(60) Provisional application No. 62/233,955, filed on Sep. 28, 2015.

(51) Int. Cl. A61B 5/145; A61B 5/00

(52) U.S. Cl. CPC A61B 5/14517; A61B 5/0008; A61B 5/14546; A61B 5/14532; A61B 5/14539; A61B 5/6833

(57) ABSTRACT

A wearable sensing platform includes sensors and circuits to sense aspects of a user's state by analyzing bodily fluids, such as sweat and/or urine, and a user's temperature. A sensor array senses a plurality of different body fluid analytes, optionally at the same time. A signal conditioner is coupled to the sensor array. The signal conditioner conditions sensor signals. An interface is configured to transmit information corresponding to the conditioned sensor signals to a remote computing device. The wearable sensing platform may include a flexible printed circuit board to enable the wearable sensing platform, or a portion thereof, to conform to a portion of the user's body.

### STATEMENT REGARDING FEDERALLY SPONSORED R&D

This invention was made with government support under Number P01 HG000205 awarded by the National Institute of Health. The government has certain rights in the invention.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### PARTIES OF JOINT RESEARCH AGREEMENT

The present disclosure was made by or on behalf of the below listed parties to a joint research agreement. The joint research agreement was in effect on or before the date the present disclosure was made and the present disclosure was made as a result of activities undertaken within the scope of the joint research agreement. The parties to the joint research agreement are: 1) The Regents of the University of California, and 2) The Board of Trustees of the Leland Stanford Junior University.

### BACKGROUND

### Field of the Invention

The present invention relates to devices that measure physiological parameters of a user.

### Description of the Related Art

Wearable electronics have been developed that can be worn by user's to continuously and closely monitor an individual's activities, such as walking and running, Such wearable electronics may include physiological sensors configured to sense certain physiological parameters of the wearer, such as heart rate, as well as motion sensors, GPS radios, and altimeters.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

An aspect of the disclosure relates to wearable biometric monitoring system comprising: a first sensor configured to sense a first sweat analyte; a second sensor configured to sense a second sweat analyte at substantially the same time as the first sensor is measuring the first sweat analyte; a signal conditioner coupled to the first sensor and the second sensor, the signal conditioner configured receive and condition sensor signals from the first sensor and the second sensor, the signal conditioner comprising one or more amplifiers and one or more filters; and an interface configured to transmit information corresponding to the conditioned sensor signals to a remote computing device.

An aspect of the disclosure relates to wearable biometric monitoring system comprising: a flexible substrate; a plurality of sweat analyte sensors affixed to the flexible substrate, the plurality of sweat analyte sensors configured to sense a plurality of different sweat analytes of a wearer at substantially the same time, the plurality of sweat analyte sensors comprising at least a first sweat analyte sensor configured to sense a metabolite and a second sweat analyte configured to sense an electrolyte; a temperature sensor configured to measure skin temperature of the wearer; a signal conditioner affixed to the flexible substrate, the signal conditioner coupled to the plurality of sweat analyte sensors, the signal conditioner configured receive and condition sensor signals from the plurality of sweat analyte sensors, the signal conditioner comprising one or more amplifiers and one or more filters; an analog and digital converter configured to convert the conditioned sensor signals from an analog domain to a digital domain, and a digital processor configured to digitally process the converted sensor signals in the digital domain, the analog and digital converter and the digital processor affixed to the flexible substrate; an interface configured to transmit information corresponding to the conditioned sensor signals to a remote computing device, the interface affixed to the flexible substrate; and a battery configured to power at least portions of the wearable biometric monitoring system.

An aspect of the disclosure relates to a method of fabricating a sweat analyte sensing system, the method comprising: patterning a flexible substrate with a sweat analyte sensor array; depositing a metal on the sweat analyte sensor array; depositing an insulating layer on the sweat analyte sensor array; defining electrode areas using photolithography and etching of the insulator; patterning a metal on the electrode areas; and forming reference electrodes corresponding to the electrode areas.

### BRIEF DESCRIPTION OF THE DRAWINGS

Embodiments will now be described with reference to the drawings summarized below. These drawings and the associated description are provided to illustrate example embodiments, and not to limit the scope of the invention.

FIGS. 1a-1d illustrate aspects of an example flexible integrated sensor array (FISA) for multiplexed perspiration analysis.

FIGS. 2a-2h illustrate example wearable sensor characterizations with respect to chronoamperometric responses of glucose sensors and lactate sensors.

FIGS. 3a-3f illustrate an example real-time perspiration analysis with respect to stationary cycling.

FIGS. 4a-4e illustrate an example hydration status analysis with respect to group outdoor running.

FIGS. 5A-5H illustrates an example flexible array fabrication process.

FIGS. 6a-6d illustrate example electrode characterizations.

FIGS. 7a-7d illustrate example user interfaces.

FIGS. 8a-8d illustrate example signal conditioning circuits for different sensor signal-types.

FIGS. 9a-9d illustrate example channel calibrations.

FIG. 9e-g illustrate aspects of an example power delivery system.

FIGS. 10a-10h illustrate example reproducibility and stability charts.

FIGS. 11a-11h illustrate a selectivity study for various example biosensors.

FIGS. 12a-12l illustrate a mechanical deformation study for various example biosensors.

FIG. 13 illustrates an example on-body real-time perspiration analysis during cycling.

FIGS. 14a-14d illustrate an example ex-situ measurement of sweat samples.

FIGS. 15a-15d illustrate example aspects of a wearable sensing system.

FIGS. 16a-16d illustrates example aspects Ca²⁺ sensor performance.

FIGS. 17a-17d illustrate example aspects of pH sensor performance.

FIGS. 18a-18f illustrate example off-body evaluations of Ca²⁺ and pH in various bodily fluids.

FIGS. 19a-19e illustrates an example real-time on-body analysis of human perspiration during a constant-load cycling.

FIG. 20 illustrates the long-term stability of Ca²⁺ and pH sensors.

FIG. 21 illustrates example aspects of sensor performance.

FIG. 22 illustrates example sensor temperature dependence.

FIGS. 23a-23d illustrate an example sensor fabrication process, schematic, and example sensor array for heavy metals analysis.

FIGS. 24a-24h illustrate an example fabrication process for flexible microsensor arrays.

FIGS. 25a-25f illustrate characterization of example Au and Bi microelectrodes for trace metal detection.

FIGS. 26a-26d illustrate an interference study and temperature compensation.

FIGS. 27a-27f illustrate various aspects related to the stability and repeatability of the microsensor arrays.

FIG. 28 illustrates various aspects of on body multiplexed trace metal detection using a microsensor array during a constant load.

FIGS. 29a-29d illustrate an example wristband and calibrated stripping voltammograms.

FIG. 30 illustrates the relationship between peak height and analyte concentration.

FIGS. 31a-31b illustrates data relating to the reproducibility of microsensor arrays.

FIGS. 32a-32d illustrates heavy metal analysis in human body fluids.

### DETAILED DESCRIPTION

Wearable sensor technologies may play a significant role in realizing personalized medicine through continuously (or periodically) monitoring an individual's health state. Disclosed herein are various example devices and sensors that can be used to sense various aspects of a user's physiological state. A wearable sensing platform is disclosed that may include some or all of the different sensors and circuits disclosed herein to sense, analyze, and report various aspects of a user's state.

To this end, human sweat is an excellent candidate for non-invasive monitoring as it contains physiologically rich information. Conventional sweat-based and other non-invasive biosensors either can only monitor a single analyte at a time or lack on-site signal processing circuitry and sensor calibration mechanisms for accurate analysis of the physiological state. Given the complexity of sweat secretion, simultaneous and multiplexed screening of target biomarkers and full system integration advantageously ensures the accuracy of measurements.

Disclosed herein is an optionally mechanically flexible and fully-integrated perspiration analysis system, including a wearable sensing platform, that simultaneously and selectively measures multiple sweat analytes, such as, by way of example, sweat metabolites (e.g. glucose and lactate) and electrolytes (e.g. sodium and potassium ions), optionally as well as the skin temperature to calibrate the sensors' response. Other sweat analyte sensors may be included as well or instead. For example, calcium, heavy metal, pH, and/or protein sensors may be included.

As discussed elsewhere herein, the panel of target analytes and skin temperature may be selected based on their informative role in understanding an individual's physiological state. Measuring and analyzing certain analytes (e.g., sodium, potassium, glucose, lactate, skin temperature, heavy metals, pH, etc.) may then be used to detect and monitor various physiological conditions. For example, excessive loss of sodium and potassium in sweat could result in hyponatremia, hypokalemia, muscle cramps or dehydration. Sweat sodium and potassium could be useful biomarkers for electrolyte imbalance and Cystic Fibrosis diagnosis. Sweat glucose comes from blood glucose. Thus, glucose monitoring is desirable in managing diabetes, and several studies have reported that sweat glucose levels are correlated with blood glucose levels. As such, sweat glucose sensing may serve as a non-invasive way for blood glucose monitoring.

Sweat lactate analysis may be helpful for many potential clinical applications. For example, sweat lactate has been shown to potentially be a very useful early warning indicator of pressure ischemia. Sweat lactate may also be used to monitor physical performance since lactate is a product of anaerobic metabolism. If there is an adequate correlation between blood and sweat lactate levels, the detection of sweat lactate may offer a non-invasive way for blood lactate monitoring. There are also reports on using sweat lactate as a biomarker for panic disorder or Frey's syndrome. Skin temperature is clinically informative of a variety of diseases and skin injuries such as pressure ulcers. Skin temperature is an effective indicator of human sensations and provides significant clinical information about cardiovascular health, cognitive state and malignancy. Additionally, skin temperature measurements may be used to compensate for and to reduce or eliminate the influence of temperature variation on the chemical sensors' readings, optionally through a built-in signal processing functionality, as discussed elsewhere herein.

Aspects of the disclosure bridges the technological gap between signal transduction, conditioning, processing and wireless transmission in wearable biosensors by merging sensors (e.g., plastic-based sensors), that interface with the skin, and silicon integrated circuits consolidated on a circuit board (e.g., a flexible circuit board, which optionally be configured to be worn around or on a wrist, arm, ankle, leg, head, chest, or other body party) for complex signal processing.

The disclosed wearable system may be used to measure the detailed sweat profile of human subjects engaged in prolonged indoor and/or outdoor physical activities, and infer real-time assessment of the physiological state of the subjects. The platform enables a wide range of personalized diagnostic and physiological monitoring applications.

Wearable electronics comprise devices that can be worn or mated with human skin to continuously and closely monitor an individual's activities, without unduly interrupting or limiting the user's motions. Accordingly, as noted above, wearable biosensors may play a significant role in realizing personalized medicine due to their capability in real-time and continuous monitoring of an individual's physiological biomarkers. Current commercially available conventional wearable sensors are only capable of tracking an individual's physical activities and vital signs (e.g. heart rate), and fail to provide insight into the user's health state at molecular levels.

To gain such insight, human sweat is an excellent candidate, as similarly discussed above, as it contains physiologically and metabolically rich information that can be retrieved non-invasively. Sweat analysis is currently used for applications such as disease diagnosis, drug abuse detection, and athletic performance optimization. Disadvantageously, for these applications, the sample collection and analysis are conventionally performed separately, failing to provide a real-time profile of sweat content secretion, while requiring extensive lab analysis using bulky instrumentations. Development of wearable sweat sensors has recently been explored where a variety of biosensors were used to measure analytes of interest (see, e.g., Supplementary Table 1 below).

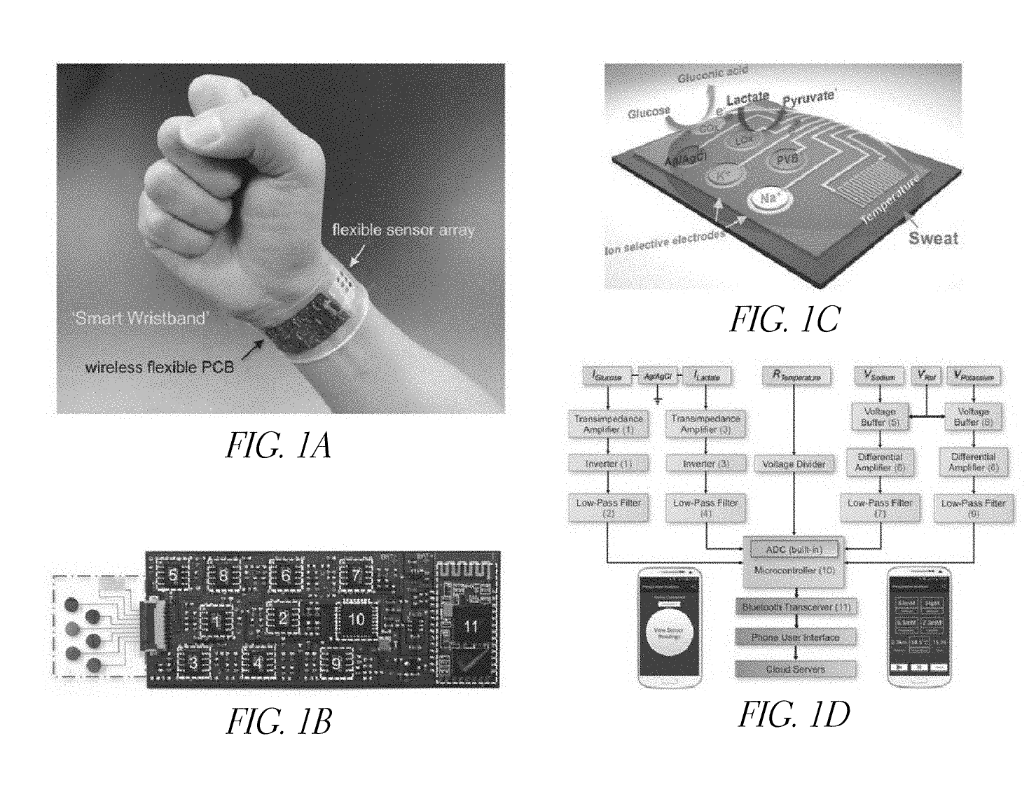

Given the complex nature of sweat and the multivariate mechanisms that are involved in its secretion process, an attractive strategy would be to devise a fully-integrated multiplexed sensing system to extract insightful information from sweat. Aligned with this vision, disclosed herein is a wearable flexible integrated sensing array (FISA) for simultaneous and selective screening of a panel of biomarkers in sweat (see, e.g., FIG. 1a). The disclosed solution bridges the existing technological gap between signal transduction (electrical signal generation by sensors), conditioning (e.g., amplification and filtering), processing (e.g., calibration and compensation), and wireless transmission in wearable biosensors by merging integrated circuit (IC) technologies (e.g., commercially available IC technologies), optionally consolidated on a single flexible printed circuit board (FPCB) (although multiple circuit boards may be used, some or all of which may be flexible), with flexible and conforming sensor technologies which may be fabricated on plastic substrates. This approach decouples the stringent mechanical requirements at the sensor level and electrical requirements at the signal conditioning, processing and transmission levels, and at the same time exploits the unique strengths offered by the underlying technologies. The independent and selective operation of individual sensors is preserved during multiplexed measurements by employing highly specific surface chemistries and electrically decoupling the operating points of each sensor's interface. This platform is a powerful tool to advance large-scale and real-time physiological and clinical studies by facilitating identification of informative biomarkers in sweat.

With reference to FIGS. 1a-1d, FIG. 1a illustrates an image of a wearable FISA on a subject's wrist which an integrated multiplexed sweat sensor array and wireless flexible printed circuit board (FPCB). FIG. 1b illustrates an image of a flattened FISA including a sensor array (in the dashed box on the left side of the FPCBP) and integrated circuit (IC) components. FIG. 1c illustrates an example schematic of the sensor array (including, in this example, glucose, lactate, sodium, potassium and temperature sensors) for multiplexed perspiration analysis. FIG. 1d illustrates an example system level block diagram of the FISA showing the signal transduction (I_{Glucose}, Ag/AgCl, R_{Temperature}, V_{Sodium}, V_{reference}, V_{Potassium}) including sensors that provide measurements utilizing current (I), resistance (R), and voltage (V). FIG. 1a includes signal conditioning of the sensor signals.

For example, the glucose sensor (with current output), has the current output amplified using a trans-impedance amplifier (1), whose output is inverted by an inverter (1), and the output of the inverter is filtered using a low-pass filter (2). By way of further example, the lactate sensor (with current output), has the current output amplified using a trans-impedance amplifier (3), whose output is inverted by an inverter (3), and the output of the inverter is filtered using a low-pass filter (4). The temperature sensor, which has a resistance that various in accordance with temperature, provides a voltage output that is divided down using a voltage divider. The sodium sensor (with voltage output), has its output buffered using a voltage buffer (5), the output of the voltage buffer (5) is amplified using a differential amplifier (6), and the output of the differential amplifier (6) is filtered using a low-pass filter (7). Potassium sensor (with voltage output), has its output buffered using a voltage buffer (8), the output of the voltage buffer (8) is amplified using a differential amplifier (6), and the output of the differential amplifier (6) is filtered using a low-pass filter (8). The analog outputs of the low-pass filters (2), (4), (7), (9), are feed into an analog-to-digital converter (ADC), which may be integrated into a processing device, such as microcontroller (10). The ADC converts the analog signals to digital signals, and the processing device may then process the digitized signals. For example, the processing device may calculate physiologic data using some or all of the data from one or more of the sensors. Optionally, the processing device and signal conditioning circuitry may be integrated into a single device.

A wireless interface (e.g., Bluetooth transceiver 10) may be used to wirelessly communicate or facilitate communication (e.g., of the processed sensor readings) to a remote device (e.g., a mobile device, such as a cell phone, tablet computer, laptop, etc., or a non-mobile device, such as a desktop computer or large screen networked television). Thus, for example, the wireless interface may facilitate or provide connectivity to, for example, relatively local external devices and/or remote devices via the Internet. The remote device may provide user interfaces that display (e.g., in real time and/or at later time) the sensor data and the remote device may upload the sensor data to a cloud system comprising one or more cloud servers or to other devices in association with a user and/or device identifier. The cloud system (or other device) may then store the sensor data (which may have been first processed by the wearable device processing system) in a data store in an account record associated with the user and/or the wearable device for later access and/or for further processing by the cloud system.

FIGS. 1a-1d will now be discussed in greater detail.

In particular, as illustrated in FIG. 1a, an example implementation of the FISA enables simultaneous and selective measurement of a panel of metabolites and electrolytes in human perspiration as well as the skin temperature (e.g., in the context of prolonged indoor and outdoor physical activities). The FISA may optionally include a mechanically flexible polyethylene terephthalate (PET) substrate. By fabricating the sensors on a mechanically flexible polyethylene terephthalate (PET) substrate, a stable sensor-skin contact is formed, while the FPCB technology is exploited to incorporate the critical signal conditioning, processing, and wireless transmission functionalities, optionally using readily available IC components (see, e.g., FIG. 1b). As discussed elsewhere herein, the panel of target analytes and skin temperature may be selected based on their informative role in understanding an individual's physiological state. For example, excessive loss of sodium and potassium in sweat could result in hyponatremia, hypokalemia, muscle cramps or dehydration; sweat glucose is reported to be metabolically related to blood glucose; sweat lactate is a sensitive marker of pressure ischemia; and skin temperature is clinically informative of a variety of diseases and skin injuries such as pressure ulcers. Additionally, skin temperature measurements may be needed to compensate and eliminate (or at least reduce) the influence of temperature variation on the chemical sensors' readings through a built-in signal processing functionality.

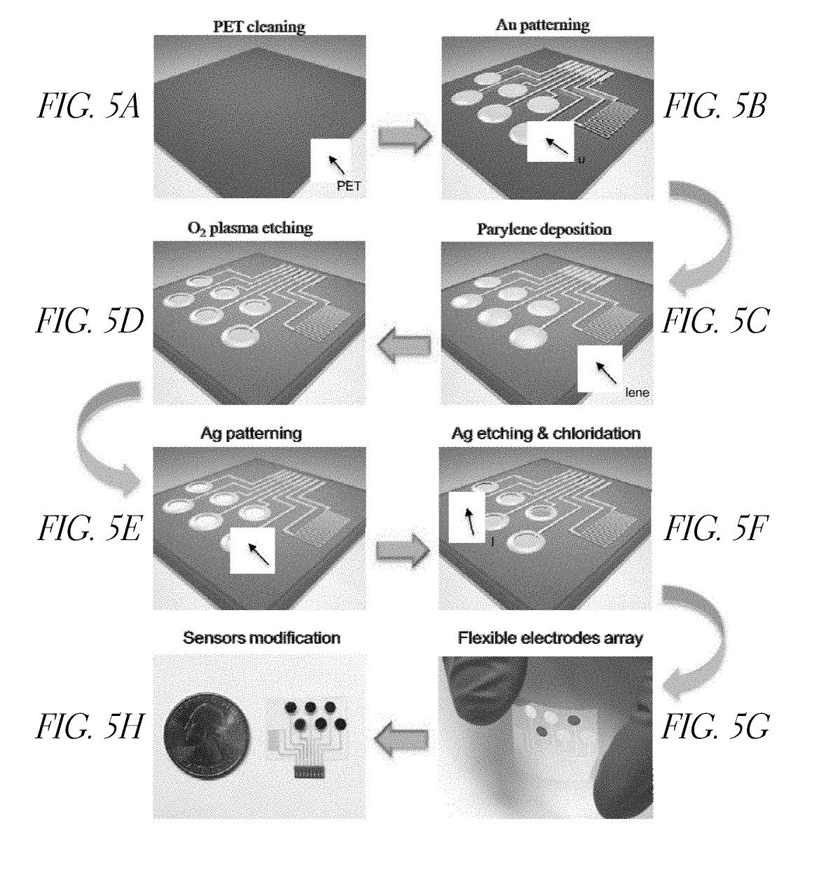

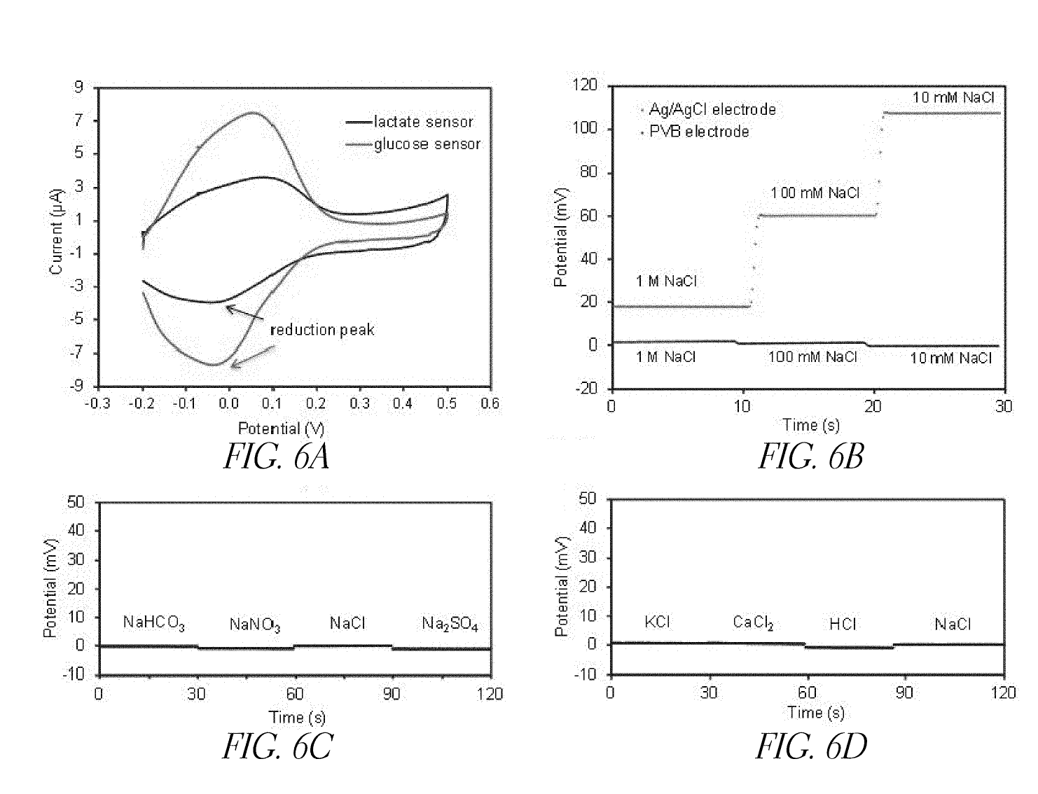

FIG. 1c illustrates the schematic of an example multiplexed sensor array, where, in this example, each electrode is 3 mm in diameter (although other diameters may be used), for sweat analysis. Example fabrication processes are detailed in the Example Techniques and Processes section of the disclosure and in FIGS. 5a-5g). Here, in this example implementation, amperometric glucose and lactate sensors (with current output) are based on glucose oxidase (GOx) and lactate oxidase (LOx) immobilized within a chitosan film. An Ag/AgCl electrode serves, in this example, as a shared reference electrode and counter electrode for both sensors, although other materials may be used. The optional use of Prussian blue (PB) as a mediator in this example minimizes the reduction potentials to ˜0 V (vs. Ag/AgCl) (see, e.g., FIG. 6a, which illustrates Cyclic voltammetry of the amperometric glucose and lactate sensors using Prussian blue as a mediator in PBS—pH 7.2. Scan range: −0.2 V to 0.5 V; scan rate: 50 mV/s), and thus eliminates the need of an external power source to activate the sensors. These enzymatic sensors autonomously generate current signals proportional to the abundance of the corresponding metabolites between the working electrode and the Ag/AgCl electrode.

The measurement of Na⁺ and K⁺ levels are facilitated through the use of ion selective electrodes (ISEs), coupled, in this example, with a polyvinyl butyral (PVB) coated reference electrode to maintain a stable potential in solutions with different ionic strengths (see, e.g., FIGS. 6b-6d). By using poly(3,4-ethylenedioxythiophene) polystyrene sulfonate (PEDOT:PSS) as an ion-to-electron transducer in the ISEs and carbon nanotubes (CNTs) in the PVB reference membrane, robust potentiometric sensors (with voltage output) can be obtained for long-term continuous measurements with negligible voltage drift. A resistance-based temperature sensor is realized, in this example, by fabricating Cr/Au metal microwires. Parylene is used, in this example, as an insulating layer to prevent electrical contact of the metal lines with skin and sweat to ensure reliable sensor reading, although other insulators may be used.

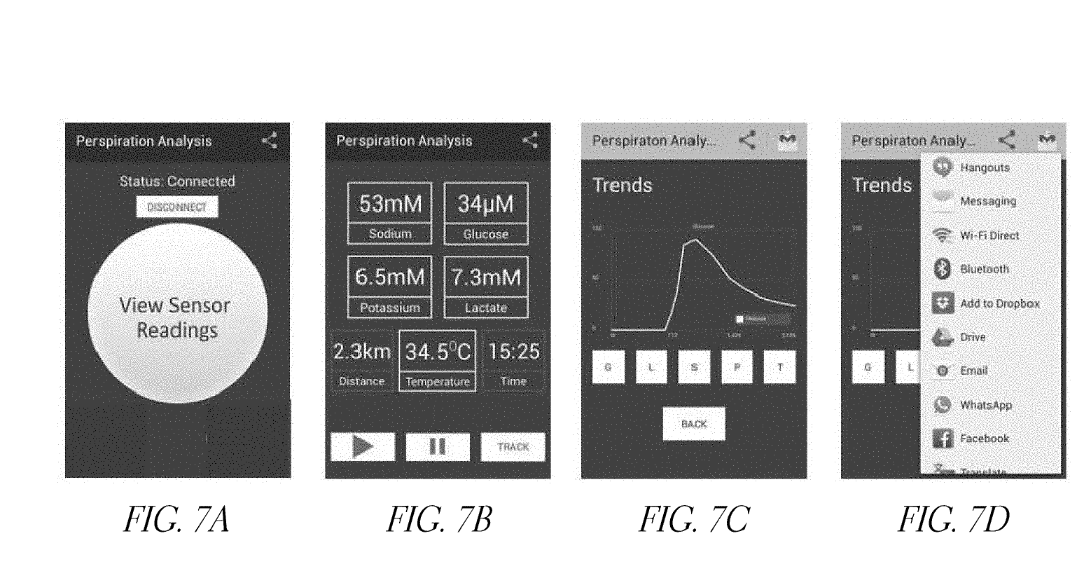

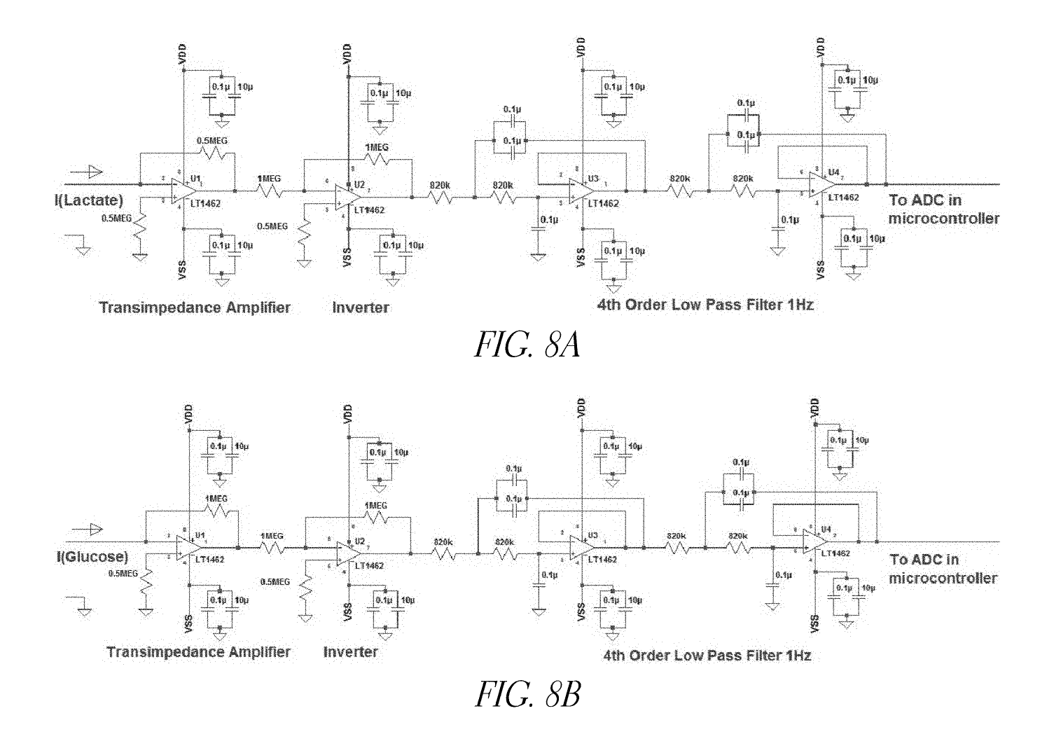

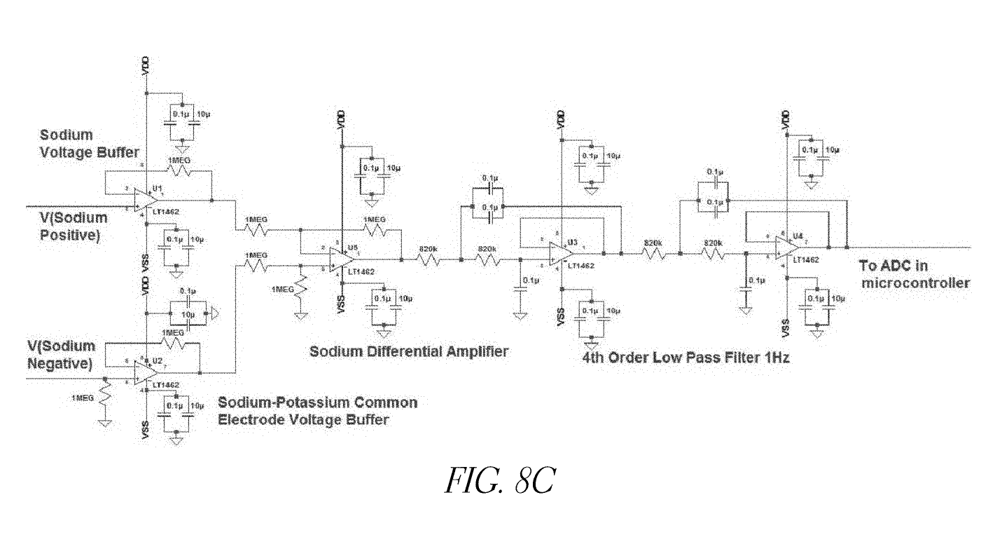

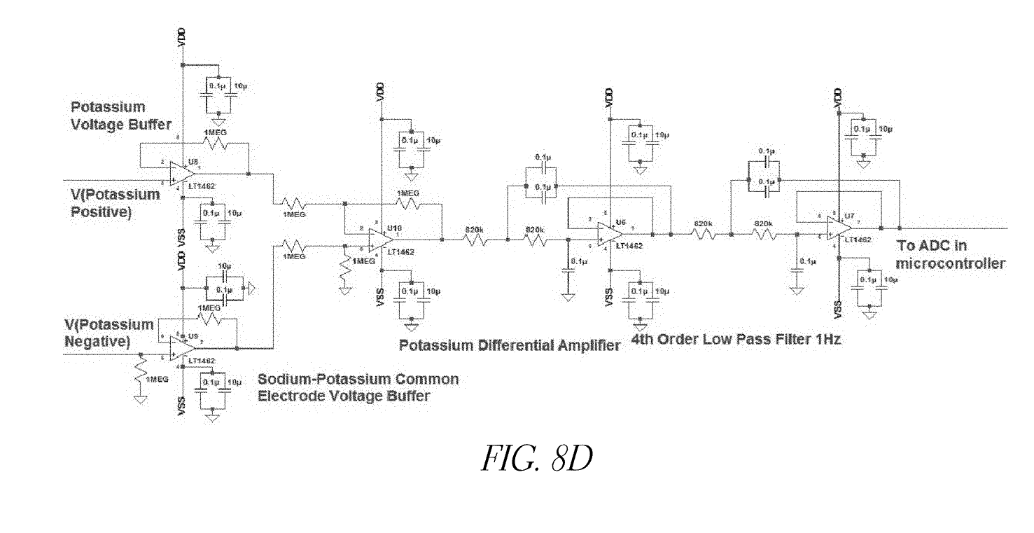

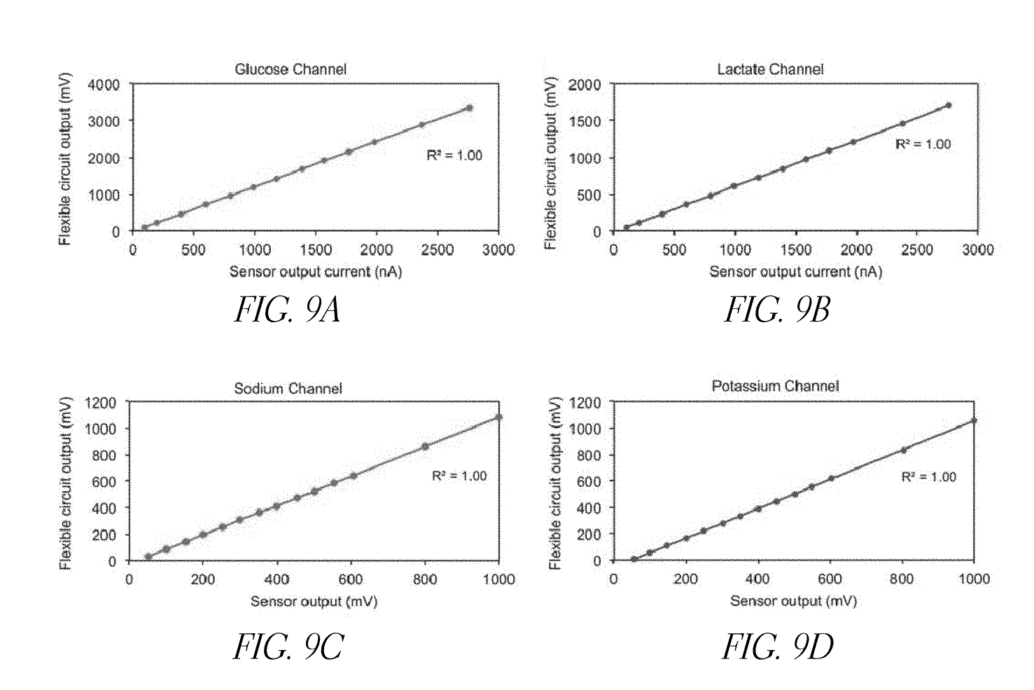

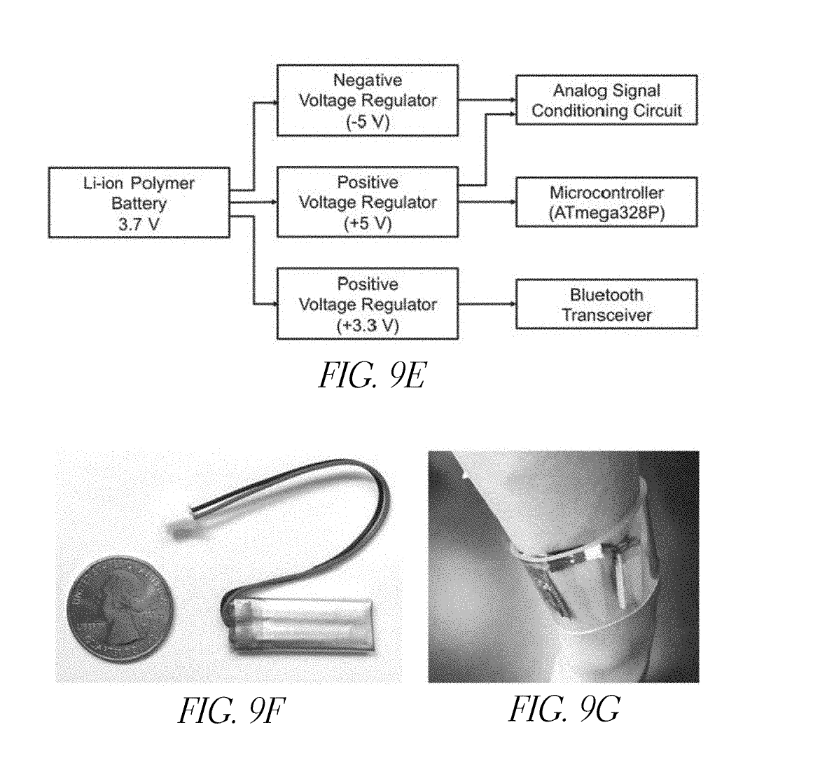

FIG. 1d illustrates an example system level overview of the signal transduction, conditioning, processing, and wireless transmission paths to facilitate multiplexed on-body measurements. The signal-conditioning path for each sensor is implemented with analog circuits and in relation to the corresponding transduced signal. The circuits in this example are configured to ensure that the final analog output of each path is finely resolved while staying within the input voltage range of the analog-to-digital converter (ADC) and/or a signal multiplexer used to multiplex signals to the ADC. Furthermore, a processing device's (e.g., a microcontroller) computational and serial communication capabilities are utilized to calibrate, compensate, and relay the conditioned signals to an interface, which may include an on-board wireless transceiver (e.g., Bluetooth, Bluetooth LP, other personal area network (PAN) interface, Wi-Fi, etc.) and/or a wired interface configured to receive a cable connector. The transceiver facilitates wireless data transmission to a mobile handset, other mobile device, or non-mobile device (e.g., equipped with a Bluetooth or other wireless interface that is compatible with the FISA wireless transceiver) with a mobile application (FIGS. 7a-7d), containing a user-friendly interface for sharing (through email, SMS, a social network platform, etc.) or uploading the data to cloud servers online. The circuit design, calibration, and power delivery diagram of the FISA are described in the Example Techniques and Processes section of the disclosure, and in FIGS. 8a-8d and 9a-9d.

Optionally, the FISA may include a display (e.g., an LCD, OLED, e-ink, or other display). The display may be coupled to the processor and may display various sensor measurements, alerts, and/or information derived from the sensor measurements. For example, the processor may utilize the display to present information on wearer dehydration, and the likelihood or presence of hyponatremia, hypokalemia, muscle cramps, ischemia, and/or pressure ulcers. The information may be displayed in an association with corresponding icons (e.g., alert icons indicating that the user is or is about to suffer an adverse physiological condition). Similarly, the mobile application may be configured to determine and/or display, via the mobile device, similar information. The display may be touch sensitive and/or the FISA may include physical controls, such as physical buttons or knobs. The wearable device may include a microphone configured to receive voice commands and may include a speaker to provide audible confirmation of the voice commands. The wearer may command the FISA to display the information measured and/or determined via analysis via the touch display, voice commands, and/or the physical controls.

Testing of an example implementation of the FISA was performed. The performance of each sensor was monitored separately with respective analyte solutions.

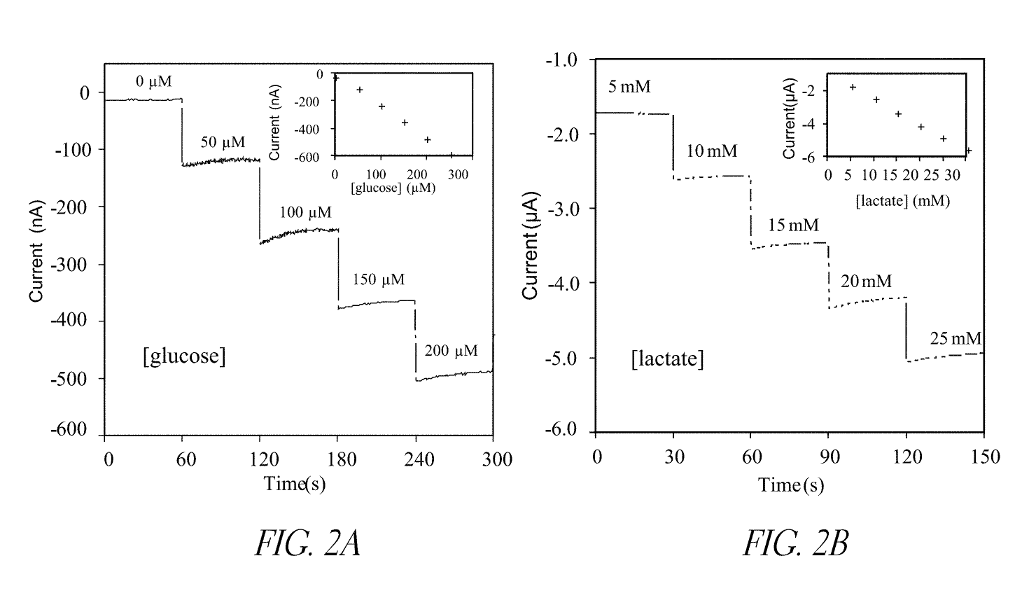

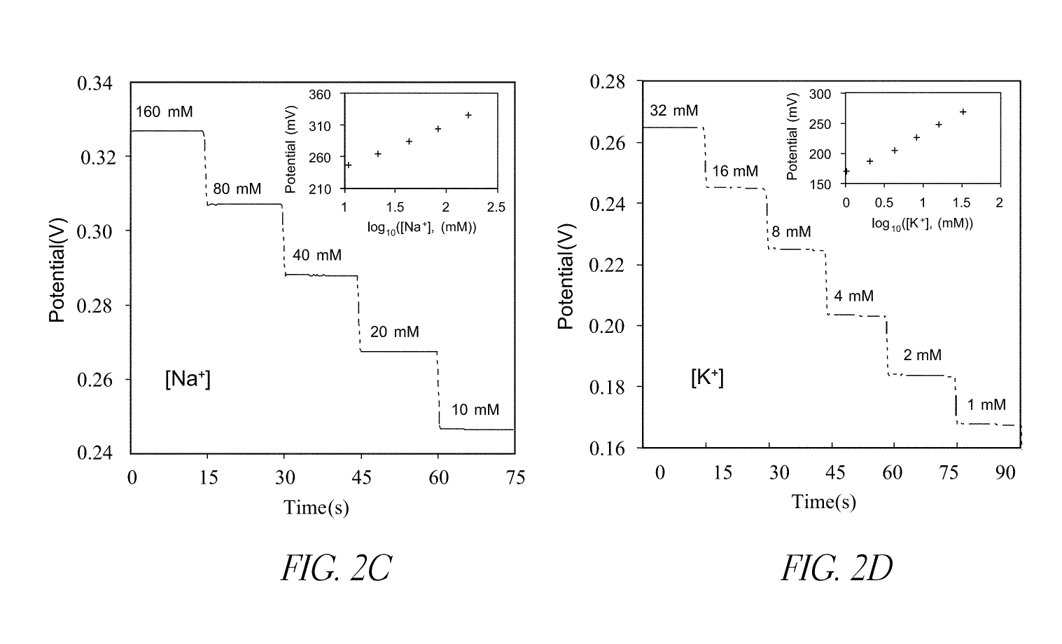

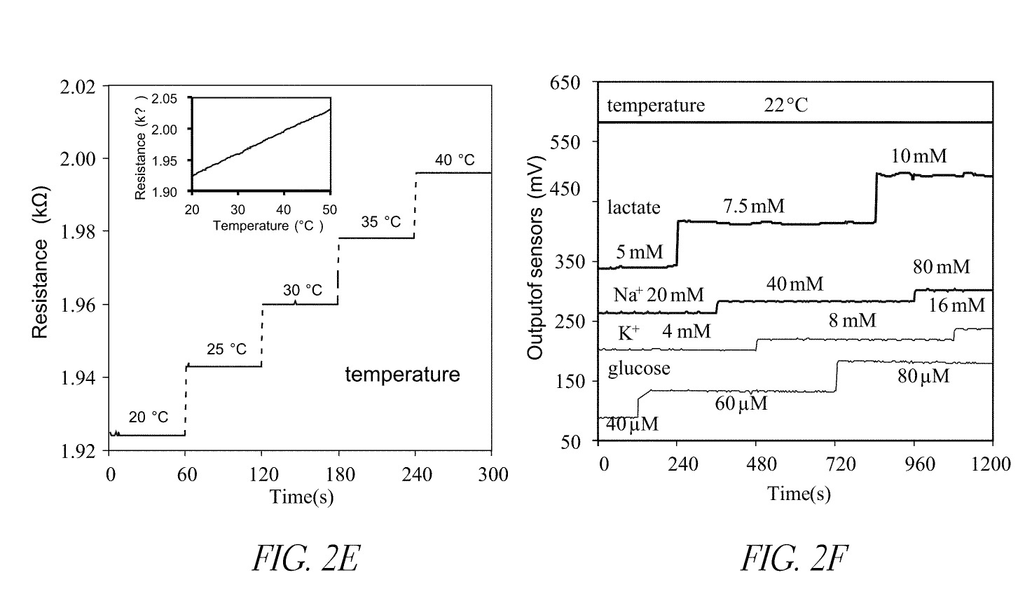

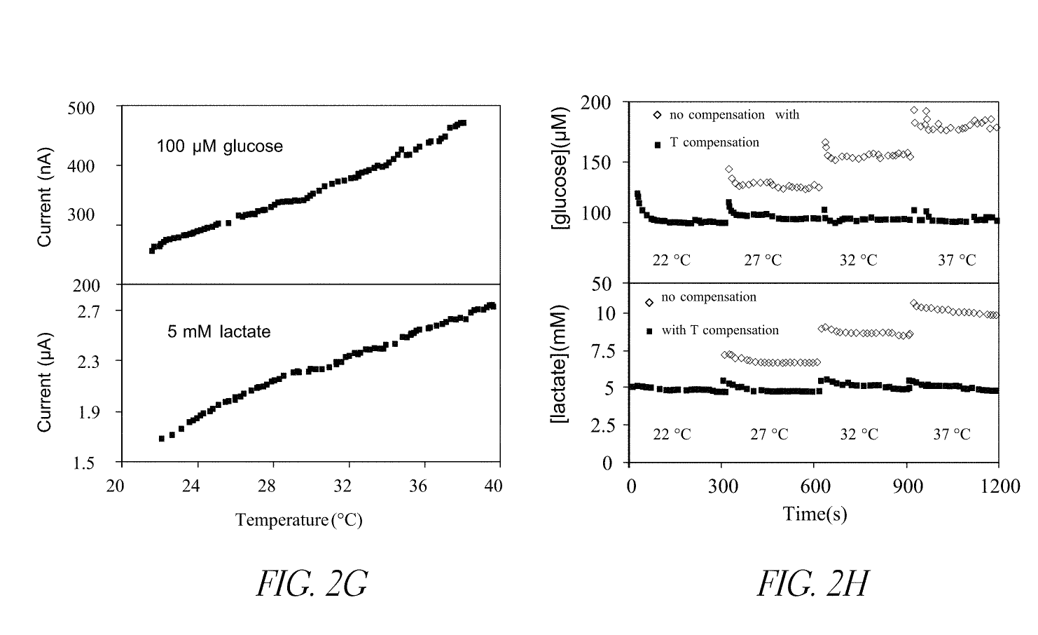

FIGS. 2a-2h illustrate, via current versus time plots, experimental characterizations of the wearable sensors, including the chronoamperometric responses of the glucose sensors (FIG. 2a) and the lactate sensors (FIG. 2b) to the respective analyte solutions in phosphate-buffered saline (PBS); the open circuit potential responses of the sodium sensors (FIG. 2c) and the potassium sensors (FIG. 2d) in NaCl and KCl solutions, via voltage versus time plots; the response, via resistance versus time plots, of the temperature sensor to temperature changes (20-40° C.) in PBS. Insets in FIG. 2a-2e illustrate the corresponding calibration plots of the sensors. In taking the corresponding measurements, data recording was paused for 30 seconds for each solution change with respect to FIGS. 2a-2e. FIG. 2f illustrates system level interference studies of the sensor array, via sensor output (in mV) versus time plots. FIG. 2g illustrates, via a current versus temperature plot, the influence of temperature on the responses of the glucose and lactate sensors. FIG. 2h illustrates system level real-time temperature (T) compensation for the glucose and lactate sensors in 100 μM glucose and 5 mM lactate solutions respectively.

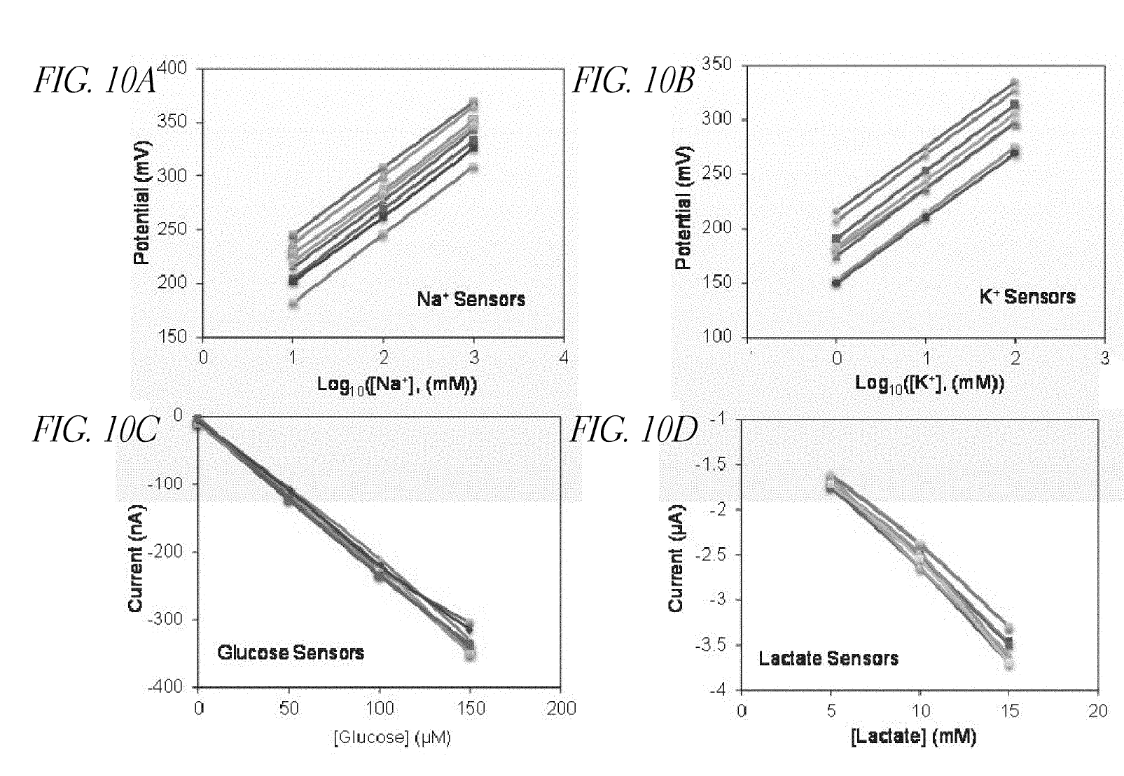

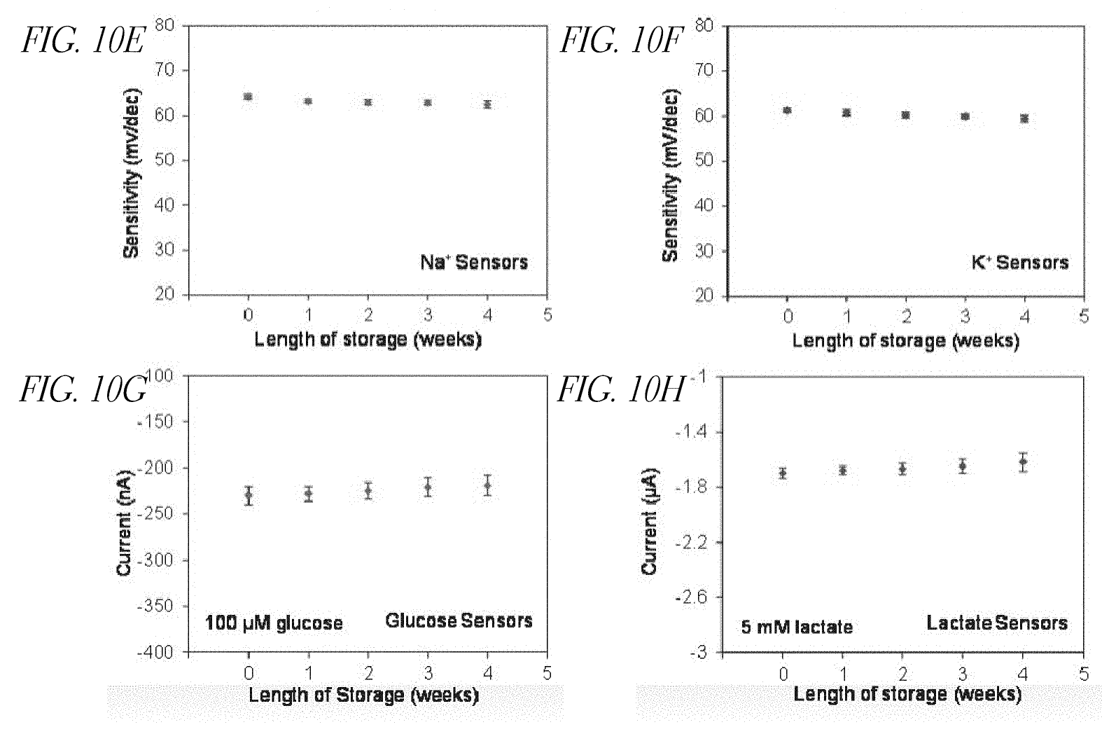

In particular, FIGS. 2a and 2b show representative current responses of the glucose and lactate sensors, measured chronoamperometrically in 0-200 μM glucose solutions and 0-30 mM lactate solutions, respectively. A linear relationship between current and analyte concentrations with sensitivities of 2.35 nA/μM for glucose sensors and 220 nA/mM for lactate sensors was observed. FIGS. 2c and 2d illustrate the open circuit potentials of Na⁺ and K⁺ sensors in the electrolyte solutions with physiologically relevant concentrations of 10-160 mM Na⁺ and 1-32 mM K⁺ respectively. Both ion-selective sensors show a near-Nerstian behavior with sensitivities of 64.2 and 61.3 mV per decade of concentration for Na⁺ and K⁺ sensors, respectively. Results of repeatability and long-term stability studies indicate that the sensitivities of the biosensors are consistent over a period of at least 4 weeks (FIGS. 10a-10h). FIG. 2e displays the linear response of the resistive temperature sensor in the physiological skin temperature range of 20-40° C. with a sensitivity of ˜0.18% per ° C. (normalized to the resistance at 20° C.).

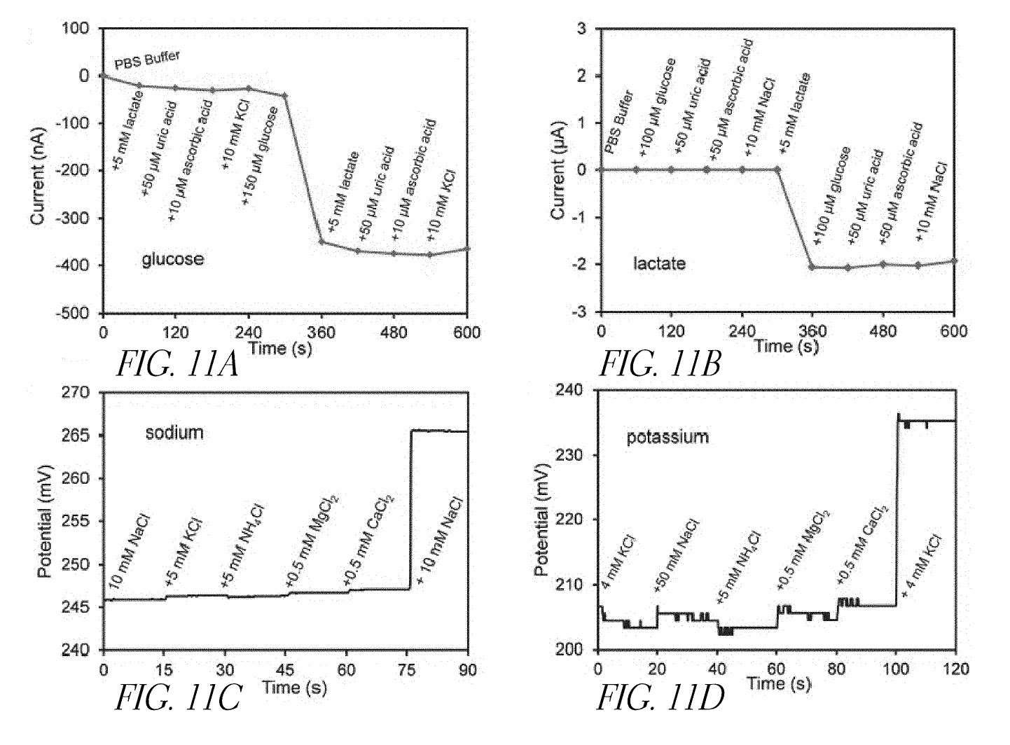

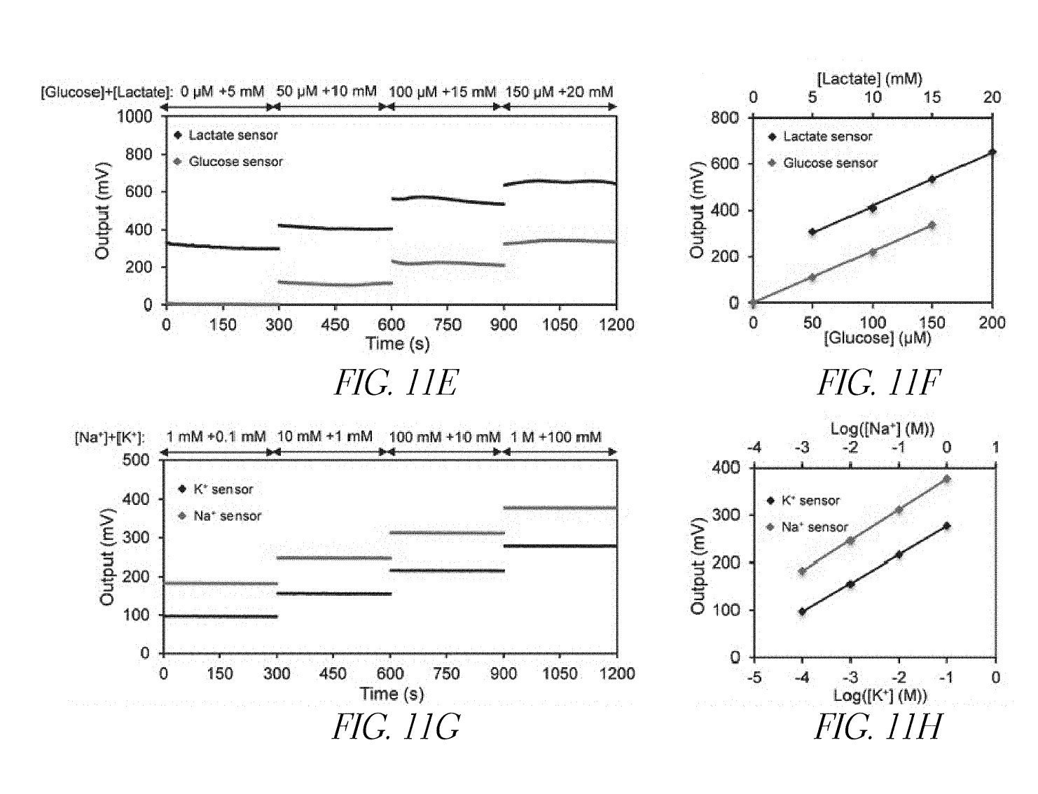

FIG. 11 illustrates an interference study for individual glucose (FIG. 11a), lactate (FIG. 11b), sodium (FIG. 11c) and potassium (FIG. 11d) sensors using an electrochemical working station, where data recording was paused for 30 seconds for the addition of each analyte in FIG. 11c and FIG. 11d. The real-time system level interference study (FIG. 11e) and calibration plot (FIG. 11f) of the amperometric glucose and lactate sensor array with a shared solid-state Ag/AgCl reference electrode are provided. The real-time interference study (FIG. 11g) and calibration plot (FIG. 11h) of potentiometric Na⁺ and K⁺ sensor array with a shared PVB coated reference electrode are provided. Data recording was paused for 30 s for each solution change in FIG. 11e and FIG. 11g. The selectivity of sweat sensors is desirable as various electrolytes and metabolites in sweat can influence the accuracy of the sensor readings. FIGS. 11a-11d demonstrate that the presence of non-target electrolytes and metabolites has negligible interference on the response of each sensor. When five different sensor-types are integrated in the FISA (a glucose sensor, a lactate sensor, a sodium sensor, a potassium sensor, and a temperature sensor in this example), simultaneous system level measurements maintain excellent selectivity upon varying concentrations of each analyte (see, e.g., FIG. 2f and FIGS. 11e-11h). Although temperature has minimal effect on the potentiometric sensors, it significantly influences the performance of the enzymatic sensors. In FIGS. 11g-11g, the graph for the K⁺ sensor is above that of the Na⁺ sensor. In FIGS. 11e-11f, the graph for the lactate sensor is above that of the glucose sensor.

FIG. 2g demonstrates that the responses of glucose and lactate sensors increase rapidly upon elevation of solution temperature from 22 to 40° C., reflecting the effect of increased enzyme activities. System integration enables implementing real-time compensation in order to calibrate the sensor readings based at least in part on temperature variations. Because the response of glucose and lactate for a given concentration is nearly linear with temperature, the compensation can be approximately liner as well. FIG. 2h illustrates that with the increase of temperature, the uncompensated sensor readouts can lead to significant overestimation of the actual concentration of the given glucose and lactate solutions, while the temperature compensation enables accurate and consistent readings. This calibration strategy represents the advantages of system level integration for wearable sensors.

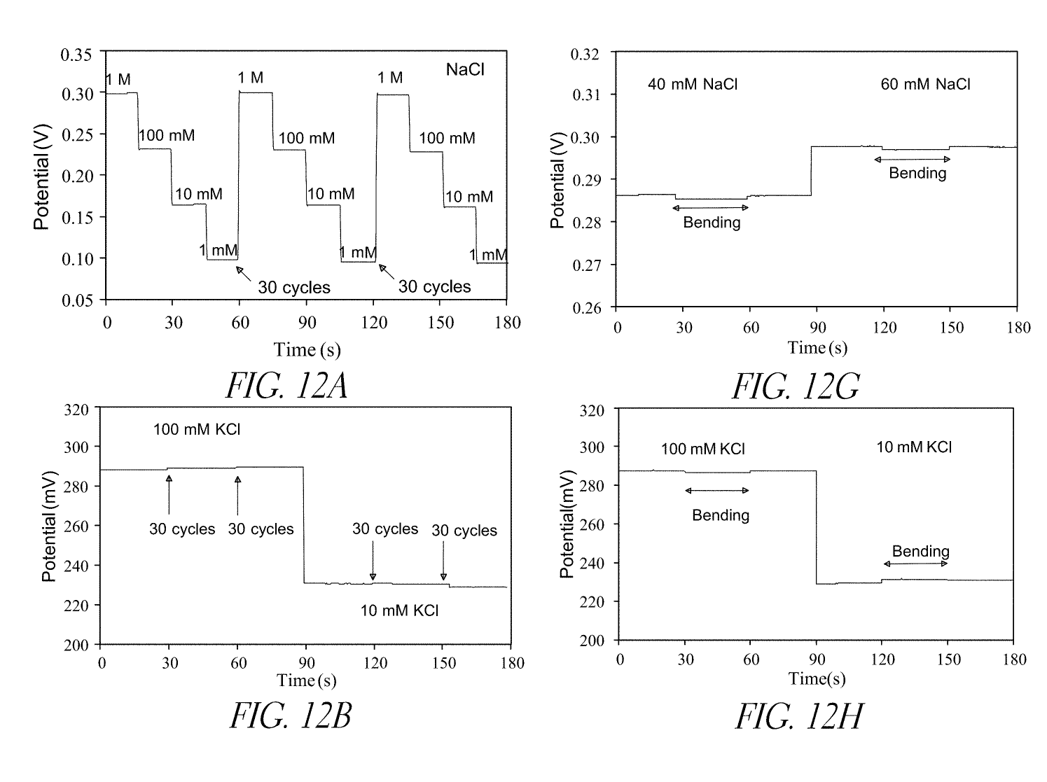

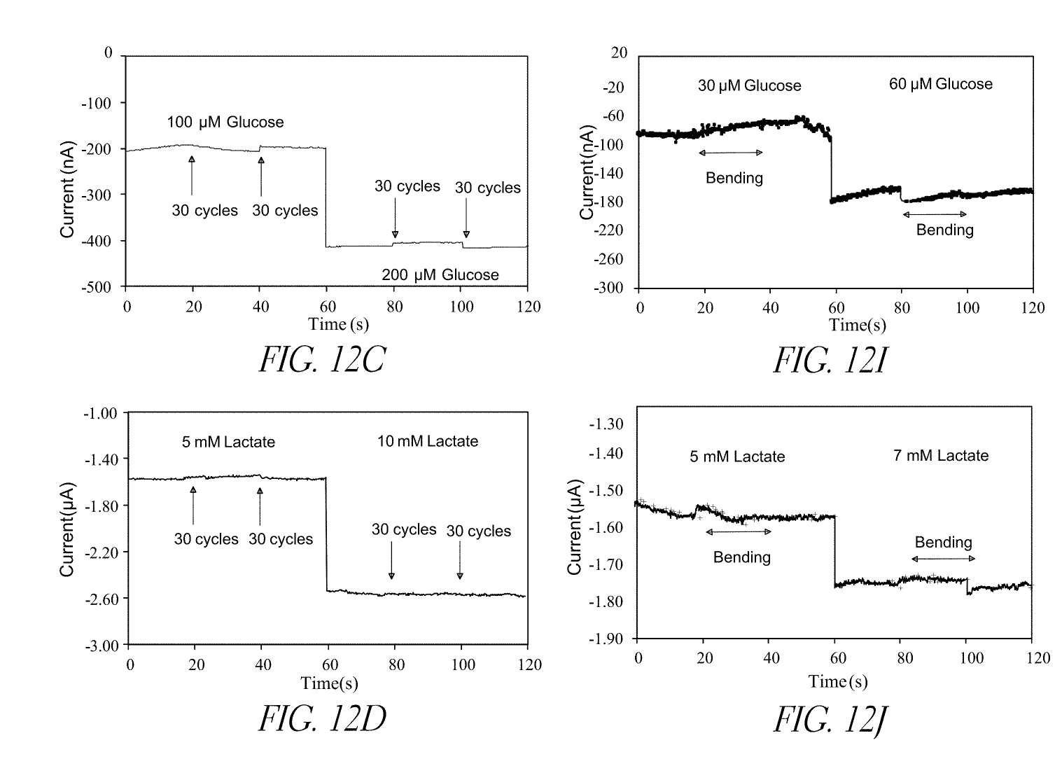

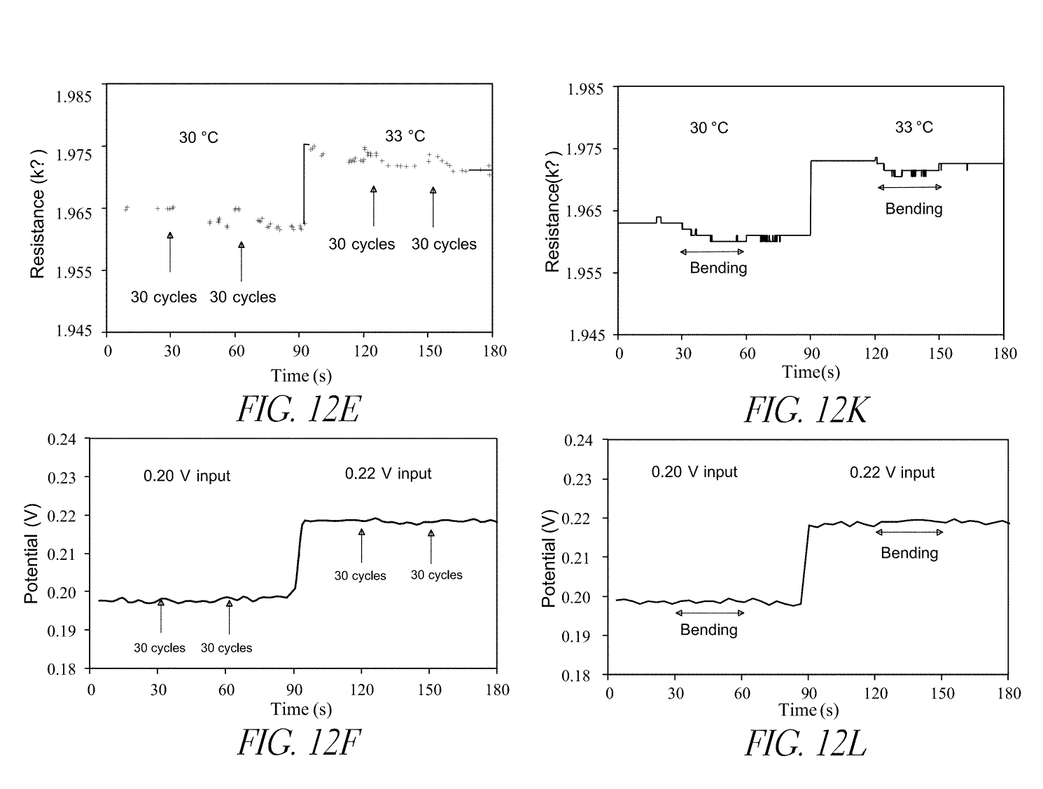

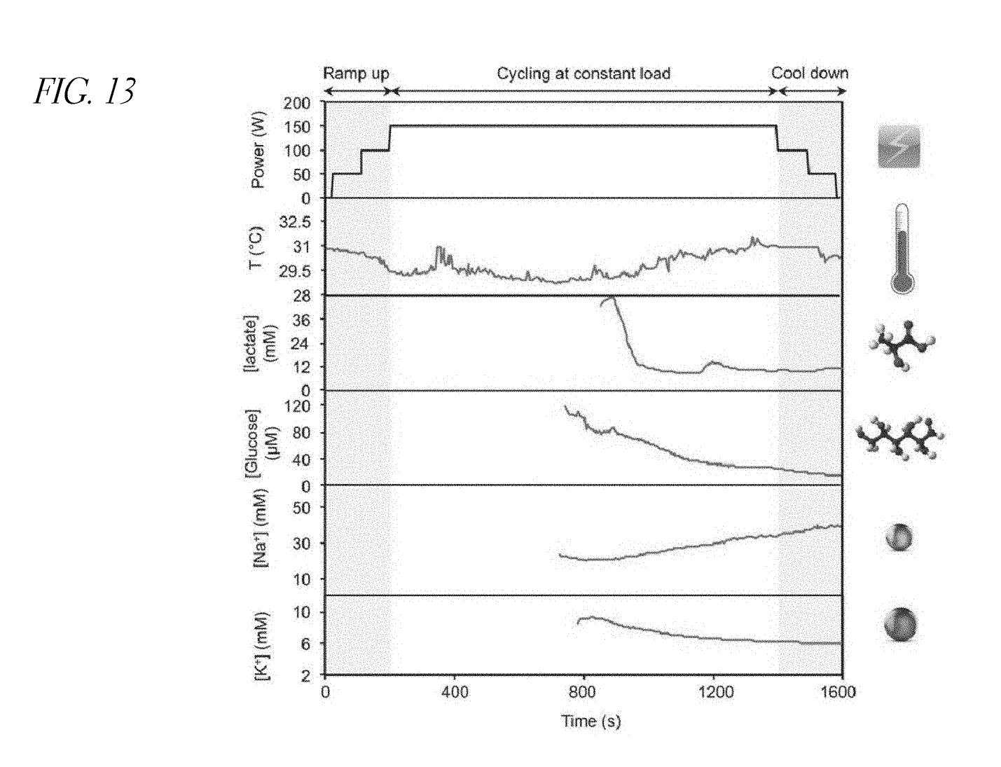

It is desirable for wearable devices to have the ability of withstanding stress from daily human wear and physical exercise in order to be utilized on a daily basis by typical users. A study on mechanical deformation conducted by monitoring the performance of both the sensor array and the FPCB before, during, and after bending (radii of curvature are 1.5 cm and 3 cm, respectively) (see, e.g., FIGS. 12a-12d) depicts minimal output changes in the FISA's responses. In particular, the responses of the sodium (FIG. 12a), potassium (FIG. 12b), glucose (FIG. 12c), lactate (FIG. 12d), temperature (FIG. 12e) sensors and FPCB (FIG. 12f) after 0, 30, and 60 cycles of bending are illustrated. The responses of the sodium (FIG. 12g), potassium (FIG. 12h), glucose (FIG. 12i), lactate (FIG. 12j), and temperature (FIG. 12k) sensors and FPCB (FIG. 12l) during bending are illustrated. The radii of curvature for the bending study of sensors and FPCB were 1.5 cm and 3 cm, respectively. Data recording was paused for 30 s to change the conditions and settings.

The FISAs can be configured to be comfortably worn on various body parts, including, for example, the forehead, wrists, and/or arms.

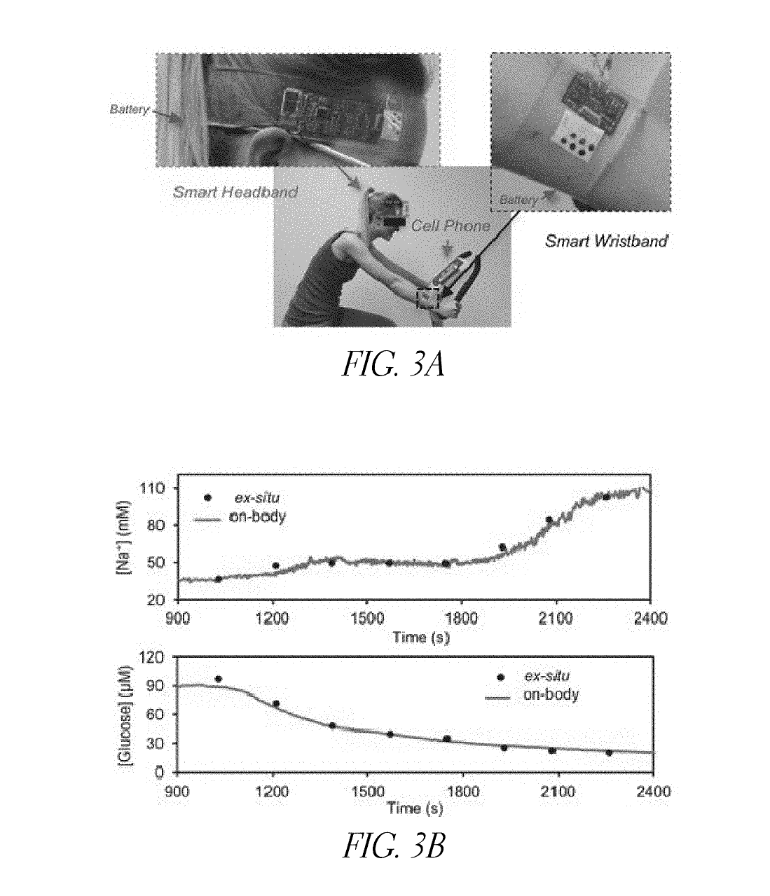

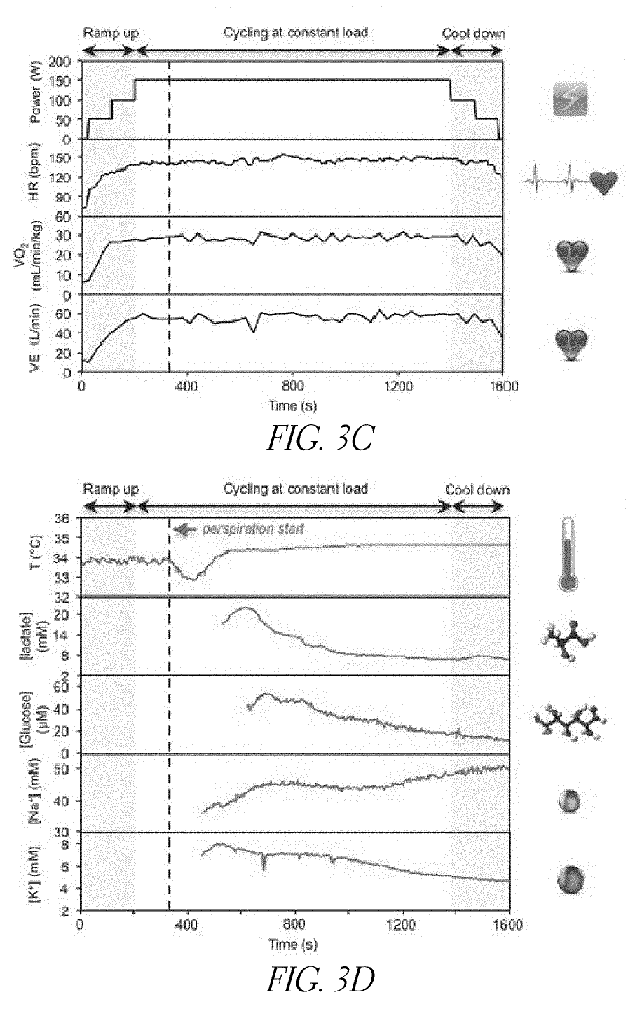

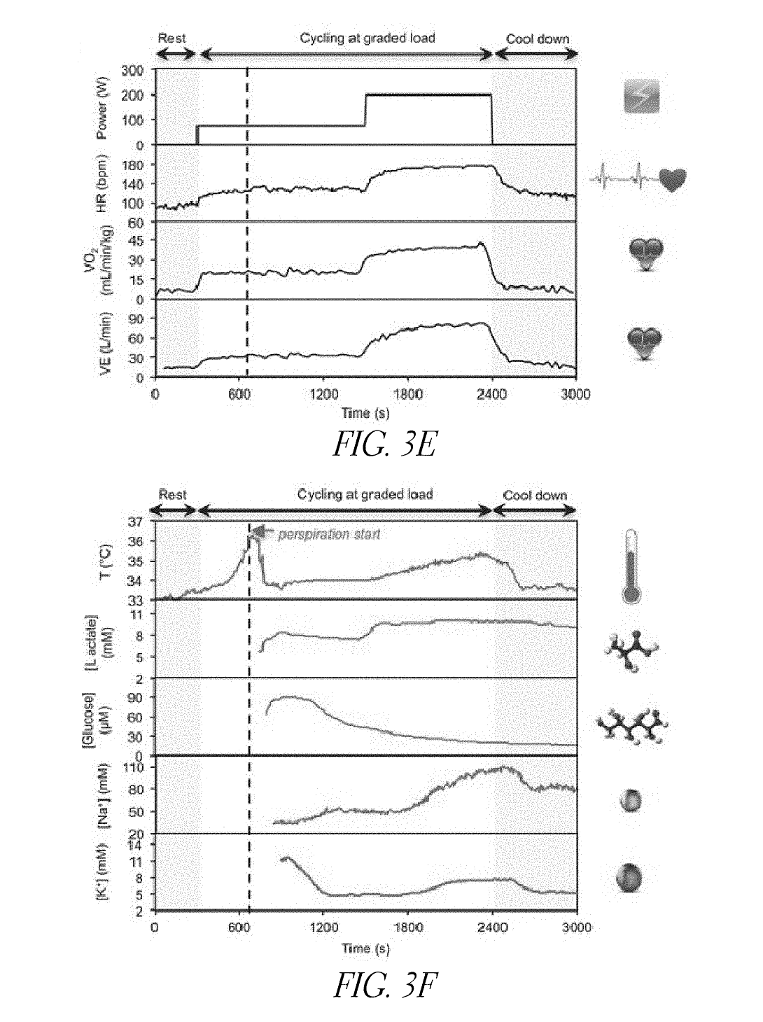

FIGS. 3a-3f illustrates aspects of on-body real-time perspiration analysis performed during stationary cycling. With reference to FIG. 3a, an example depiction is provided of a subject wearing a smart headband and a smart wristband, that incorporate technologies and materials disclosed herein, during stationary cycling. With reference to FIG. 3b, depicted is a comparison of ex-situ calibration data of the sodium and glucose sensors from the collected sweat samples with the on-body readings of the FISA during the stationary cycling exercise detailed in FIG. 3f. FIGS. 3c, 3d, illustrate, for constant-load exercise at 150 W: power output (PO), heart rate (HR), oxygen consumption (VO₂) and minute ventilation (VE) as measured by external monitoring systems (FIG. 3c) and the real-time sweat analysis results of the FISA worn on a subject's forehead (FIG. 3d). FIGS. 3e, 3f, illustrate, for graded-load exercise, involving a dramatic power increase from 75 W to 200 W: PO, HR, VO₂ and VE as measured by external monitoring systems (FIG. 3e) and the real-time analysis results using the FISA worn on a different subject's forehead (FIG. 3f). Data collection for each sensor took place when sufficient sweat sample was present.

In particular, FIG. 3a shows a human subject wearing two FISAs at the same time, packaged as a smart wristband and a smart headband, providing real-time perspiration monitoring on the wrist and forehead simultaneously during stationary leg cycling. To ensure fidelity of sensor readings, the data collection of each channel (sodium and glucose sensors in this example) took place when sufficient sweat sample was present, as evident by stabilization of sensor readings within the physiologically relevant range (see, e.g., the Example Techniques and Processes section of the disclosure). The accuracy of on-body measurements was verified through the comparison of on-body sensor readings from the forehead with ex-situ measurements from collected sweat samples (see, the plot illustrated in FIG. 3b).

Real-time physiological monitoring was performed on a subject during constant-load exercise on a cycle ergometer. In this example, the protocol involved a 3-minute ramp up, a 20-minute cycling at 150 W, and a 3-minute cool down. During the exercise, the heart rate (HR), oxygen consumption (VO₂), and minute ventilation (VE) were measured using external monitoring instruments, and were found to increase proportionally with increasing power output (PO) as shown in FIG. 3c. FIG. 3d illustrates the corresponding real-time measurements on the subject's forehead using a FISA. The skin temperature remains constant at 34° C. up to perspiration initiation at ˜320 s. The dip in temperature at this point indicates the beginning of perspiration and evaporative cooling. With continuation of perspiration process, skin temperature rises at ˜400 s because of muscle heat conductance to skin and then remains stable, while both sweat lactate and glucose decrease gradually (square brackets represent concentration). The decreases in sweat lactate and glucose are expected due to the dilution effect caused by an increase in sweat rate which is visually observed as exercise continues. However, lactate becomes relatively stable after 1100 seconds, indicating the stabilization of physiological responses to continuous, sub-maximal constant exercise power output. Sweat [Na⁺] increases and [K⁺] decreases in the beginning of perspiration, in line with the previous ex-situ studies from the collected sweat samples. Both [Na⁺] and [K⁺] stabilize as the cycling continues. By wearing FISA at different parts of the body, the site-specific variations in electrolytes and metabolites levels can also be monitored and studied simultaneously.

Sweat analyte levels on the wrist follow similar trends but with different concentrations from the forehead (see, e.g., FIG. 13, which illustrates on-body real-time perspiration analysis during stationary cycling using the FISA on a subject's wrist during stationary cycling, with conditions, same as FIGS. 3c and 3d). In this case, because the subject had a lower sweat rate on the wrist, the sensors were activated at a later time.

The physiological response of the subjects due to a sudden change in exercise intensity was also investigated in a graded-load exercise which involved a 5-minute rest, a 20-minute cycling at 75 W followed by a cycling at 200 W PO until volitional fatigue, and a 10-minute recovery (FIGS. 3e and 3f). As demonstrated in FIG. 3e, the dramatic increase in the exercise PO from 75 W to 200 W immediately leads to abrupt elevations of HR, VE and VO₂. Responses of the FISA during 75 W PO follow the profiles similar as those observed during the constant-load study. After the power is raised, the sweat rate visibly increases, followed by a dramatic increase in skin temperature and sweat [Na⁺] as well as a slight increase in [K⁺] (in 3 of 7 subjects, [K⁺] remained stable). The relatively stable behavior of [K+] is reasonable considering its passive ion partitioning mechanism 13. With the cessation of exercise, these physiological responses decrease and then remain stable. No apparent difference is observed for glucose at different PO settings, a finding consistent with the response of blood glucose to graded, short-term exercise. The change of lactate, on the other hand, varies between subjects. This observation can be attributed to the increase in both lactate excretion rate and sweat rate upon the increase of the workload.

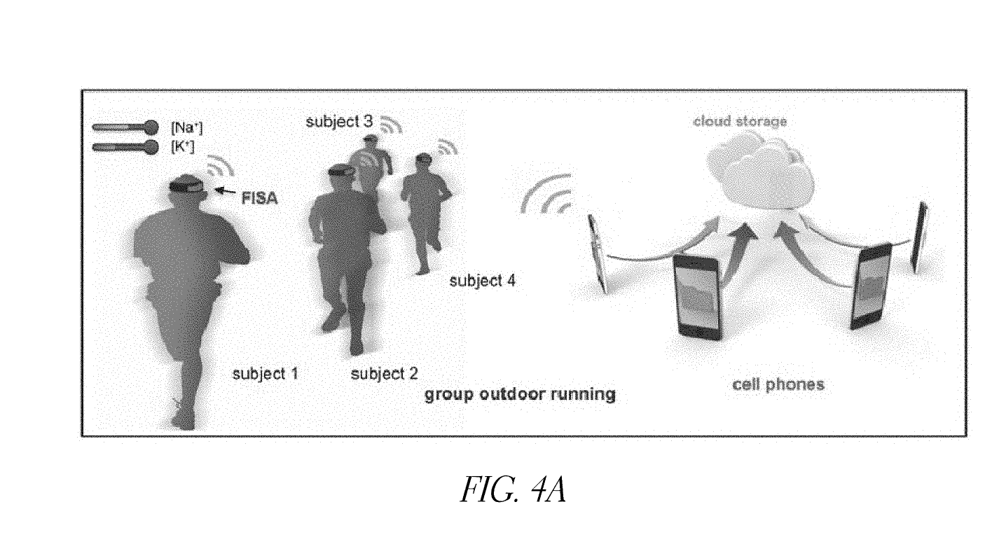

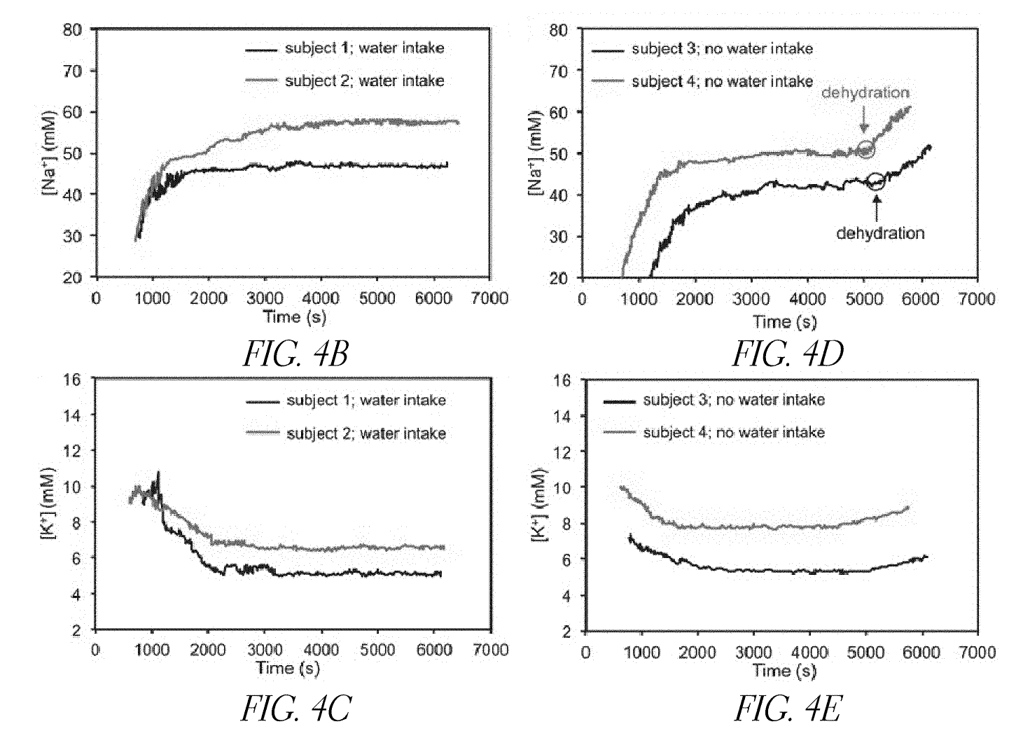

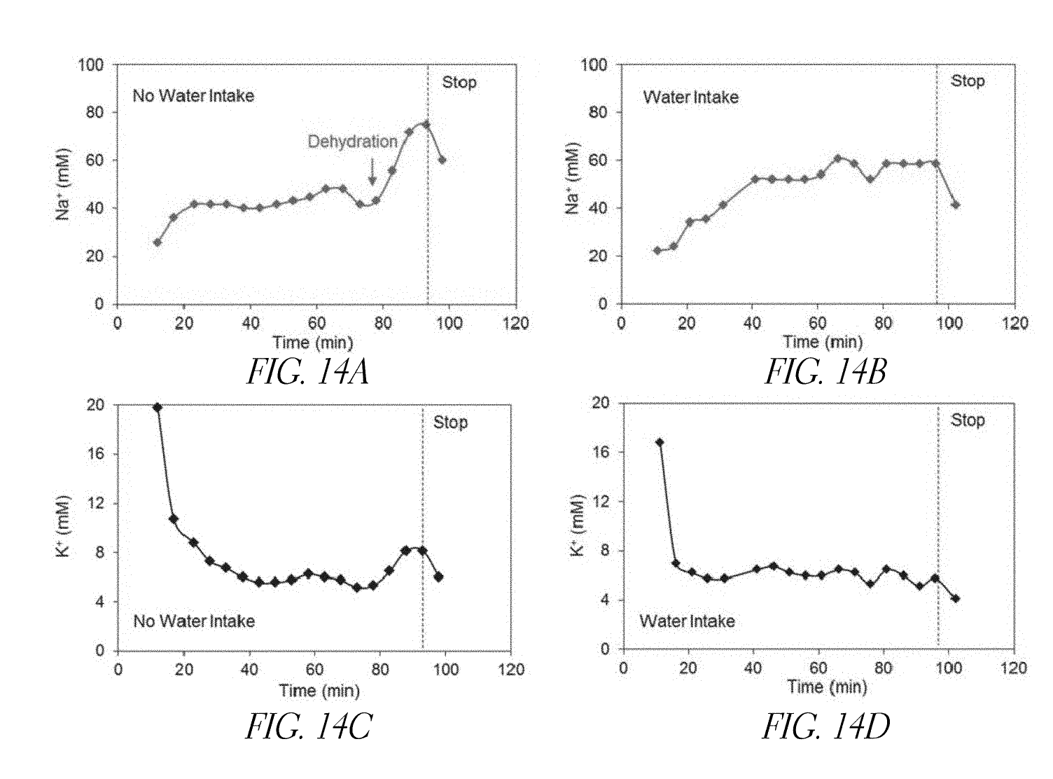

Monitoring hydration status is highly desirable for athletes, as fluid deficit impairs endurance performance and increases carbohydrate reliance. To evaluate the utility of FISA in identification of the dehydration status as an effective and non-invasive approach, real-time sweat [Na⁺] and [K⁺] measurements were conducted simultaneously on a group of subjects engaged in prolonged outdoor running trials (FIG. 4a). FIGS. 4b and 4c show (for subjects 1 and 2, with the graph for subject 1 above that of subject 2) that sweat [Na⁺] and [K⁺] are stable throughout running in euhydration trials (with 150 mL/5 minute water intake) after the initial [Na⁺] increase and [K⁺] decrease. On the other hand, a significant increase in sweat [Na⁺] and a relatively smaller increase in sweat [K⁺] (no clear increase on [K⁺] was observed in 2 out of 6 subjects) were observed in dehydration trials (without water intake) after 80 minutes when subjects had lost a large amount of water (˜2.5% (w/w) dehydration) (FIGS. 4d and 4e for subjects 3 and 4, with the graph for subject 3 above that of subject 4). Ex-situ measurements of [Na⁺] and [K⁺] from collected sweat samples in FIGS. 14a-14d also show similar phenomena.

FIGS. 14a-14d show ex-situ measurement of collected sweat samples using the FISA from a subject during stationary cycling at 150 W, including: the ex-situ results of [Na+] (FIG. 14a) and [K+] (FIG. 14c) from the collected sweat samples from the subject's forehead without water intake (˜2.5% (w/w) dehydration); the ex-situ results of [Na+] (FIG. 14b) and [K+] (FIG. 14d) from the collected sweat samples of the subject's forehead with water intake (150 mL/5 min). These trends are likely caused by increased serum [Na⁺] and [K⁺] with dehydration and increased neural stimulation, a conclusion in agreement with previous ex-situ sweat analyses. Thus, sweat [Na⁺] can potentially serve as an important biomarker for dehydration monitoring. The disclosed wearable platform can enable new fundamental physiology studies and new trends could be observed upon further on-body evaluation. Thus, the wearable platform may be configured to determine, from sensor measurements, the likelihood or presence of wearer dehydration, hyponatremia, hypokalemia, muscle cramps, ischemia, and/or pressure ulcers, and may provide corresponding alerts and reports.

Here, in the illustrated example implementation, skin-conforming plastic-based sensors (5 different sensors in this example, although additional or fewer sensors may be used) and IC components (e.g., conventional commercially available or custom IC components, including more than 10 chips in this example) are merged at high level of integration, to not only measure the output of an array of multiplexed and selective sensors, but to also through signal processing obtain accurate assessment of physiological state of the human subjects. The envisioned application could not have been realized by either of the technologies alone due to their respective inherent limitations. The plastic-based device technologies lack the ability to implement sophisticated electronic functionalities for critical signal conditioning and processing. On the other hand, the silicon IC technology does not provide sufficiently large active areas nor intimate contact to skin needed to achieve stable and sensitive on-body measurements. Importantly, the entire system may be mechanically flexible and self-sustained, thus delivering a practical wearable sensor technology that can be used for prolonged indoor and outdoor physical activities. The same platform can be configured for in-situ analyses of other biomarkers within sweat and other human fluid samples to facilitate personalized and real-time physiological and clinical investigations. The large data sets that are collected through such studies along with voluntary community participation would enable application of data mining techniques to generate predictive algorithms for understanding the health status and clinical needs of individuals and the society as a whole.

### Example Techniques and Processes

Certain example optional techniques and processes, and related optional example materials, circuits, dimensions, and amounts, will now be described. In addition certain measured results of fabricated components and devices will also be described. It is understood that other techniques and processes, and related optional example materials, circuits, dimensions, and amounts, may be used.

Example Materials. Selectophore™ grade sodium ionophore X, bis(2-ethylehexyl) sebacate (DOS), sodium tetrakis[3,5-bis(trifluoromethyl)phenyl] borate (Na-TFPB), high-molecular weight polyvinyl chloride (PVC), tetrahydrofuran (THF), valinomycin (potassium ionophore), sodium tetraphenylborate (NaTPB), cyclohexanone (CHA), polyvinyl butyral resin BUTVAR B-98 (PVB), sodium chloride (NaCl), 3,4-ethylenedioxythiophene (EDOT), poly(sodium 4-styrenesulfonate) (NaPSS), glucose oxidase (GOx) (from Aspergillus niger), chitosan (CS), single-walled carbon nanotubes (SWCNTs), iron (III) chloride, potassium ferricyanide (III), multiwall carbon nanotubes (MWCNTs), block polymer PEO-PPO-PEO (F127), L-Lactate oxidase (LOx) (activity, >80 U/mg), phosphate buffered saline (PBS)—pH 7.2, moisture-resistant polyethylene terephthalate (PET)—100 μm thick.

Example Fabrication of electrode arrays. An example fabrication process of the electrode arrays is demonstrated in FIG. 5. In this example, acetone, isopropanol and 0₂ plasma etching were used for pet cleaning (FIG. 5a). The Cr/Au electrodes for the sensor arrays on PET were patterned by photolithography using positive photoresist (Shipley Microposit S1818) followed by 30/50 nm Cr/Au deposited via e-beam evaporation and lift-off in acetone (FIG. 5b). A 500 nm parylene C insulation layer was then deposited in a SCS Labcoter 2 Parylene Deposition System (FIG. 5c). Subsequently, photolithography was used to define the final electrode area (3 mm-diameter) followed by O₂ plasma etching for 450 s at 300 W to completely remove the parylene (FIG. 5d). E-beam evaporation was then performed to pattern 180 nm Ag on the electrode areas followed by lift-off in acetone (FIG. 5e). The Ag patterns on working electrode area were dissolved in a 6 M HNO3 solution for 1 minute (FIG. 5f). The Ag/AgCl reference electrodes were obtained by injecting 10 μL 0.1 M FeCl3 solution on top of each Ag reference electrode using micropipette for 1 minute. FIG. 5g illustrates an image of the flexible electrode array. FIG. 5h, Optical illustrates image of the multiplexed sensor array after surface modification.

Example Design of electrochemical sensors. For amperometric glucose and lactate sensors, a two-electrode system where Ag/AgCl acts as both reference and counter electrode was chosen to simplify circuit design and to facilitate system integration. The two-electrode system is a common strategy for low current electrochemical sensing. Other sensor configurations may be used. The output currents (between working electrode and Ag/AgCl reference/counter electrode) of the glucose and lactate sensors could be converted to a voltage potential through a transimpedance amplifier. amperometric sensors with larger area provide larger current signal. Considering the low concentration of glucose in sweat, the sensors are designed to be 3 mm in diameter to obtain a high current, although other diameters may be used.

Preparation of Na⁺ and K⁺ selective sensors. The Na⁺ selective membrane cocktail in this example comprises Na ionophore X (1% w/w), Na-TFPB (0.55% w/w), PVC (33% w/w), and DOS (65.45% w/w). In this example, 100 mg of the membrane cocktail was dissolved in 660 μL of THF17. The K⁺ selective membrane cocktail was composed of valinomycin (2% w/w), NaTPB (0.5%), PVC (32.7% w/w), and DOS (64.7% w/w). In this example, 100 mg of membrane cocktail was dissolved in 350 μL of CHA. The ion selective solutions were sealed and stored at 4° C. The solution for the PVB reference electrode was prepared by dissolving 79.1 mg PVB and 50 mg of NaCl into 1 mL methanol 36. 2 mg F127 and 0.2 mg MWCNTs were added into the reference solution to reduce or minimize the potential drift.

Poly(3,4-ethylenedioxythiophene) polystyrene sulfonate (PEDOT:PSS) was chosen for this example implementation as the ion-electron transducer to minimize the potential drift of the ISEs37 and deposited onto the working electrodes by galvanostatic electrochemical polymerization with an external Ag/AgCl reference electrode from a solution containing 0.01 M EDOT and 0.1 M NaPSS. A constant current of 14 μA (2 mA/cm2) was applied to produce polymerization charges of 10 mC onto each electrode.

Ion-selective membranes were then prepared by drop-casting 10 μL of the Na⁺ selective membrane cocktail and 4 μL of the K+ selective membrane cocktail onto their corresponding electrodes. The common reference electrode for the Na⁺ and K⁺ ISEs was modified by casting 10 μl of reference solution onto the Ag/AgCl electrode. The modified electrodes were left to dry overnight. The sensors could be used without pre-conditioning (with a small drift of ˜2-3 mV/h). However, to obtain the best performance for long-term continuous measurements such as dehydration studies, the ion-selective sensors were covered with a solution containing 0.1 M NaCl and 0.01 M KCl through microinjection (without contact to glucose and lactate sensors) for 1 h before measurements. This conditioning process was important to further minimize the potential drift.

Example preparation process for preparation of lactate and glucose sensors. 1% CS solution was first prepared by dissolving CS in 2% acetic acid and magnetic stirring for about 1 h; next, the CS solution was mixed with SWCNTs (2 mg/mL) by ultrasonic agitation over 30 minute to prepare a viscous CS/CNTs solution. To prepare the glucose sensors, the CS/CNTs solution was mixed thoroughly with GOx solution (10 mg/mL in PBS—pH 7.2) by a ratio of 2:1 (v/v). A Prussian blue (PB) mediator layer was deposited onto the Au electrodes by cyclic voltammetry from 0 V to 0.5 V (vs. Ag/AgCl) for 1 cycle at a scan rate of 20 mV/s in a fresh solution containing 2.5 mM FeCl3, 100 mM KCl, 2.5 mM K3Fe(CN)6, and 100 mM HCl. A thinner PB layer can provide better sensitivity which is essential for the low glucose level measurements in sweat. The glucose sensor was obtained by drop-casting 3 μL of the GOx/CS/CNTs solution onto the PB/Au electrode. For the lactate sensors, the PB mediator layer was deposited onto the Au electrodes by cyclic voltammetry from −0.5 V to 0.6 V (vs. Ag/AgCl) for 10 cycles at 50 mV/s in a fresh solution containing 2.5 mM FeCl3, 100 mM KCl, 2.5 mM K3Fe(CN)6, and 100 mM HCl. The thicker PB layer can provide a wider linear response range which is advantageous for the lactate measurement in sweat. 3 μL of CS/CNTs solution was casted onto the PB-Au electrode and dried under ambient environment; the electrode was later covered with 2 μL of LOx solution (40 mg/mL) and finally 3 μL CS/CNTs solution. The sensor arrays were allowed to dry over-night at 4° C. with no light. The solutions were stored at 4° C. when not in use.

Example signal conditioning, processing and wireless transmission circuit design. An example circuit diagram of the analog signal-conditioning block of the flexible integrated sensor array (FISA) is shown in FIG. 8. Signal conditioning circuits are illustrated for (8a) glucose, (FIG. 8b) lactate, (FIG. 8c) sodium and (FIG. 8d) potassium channels. In this example, an ATmega328P (Atmel 8-bit) microcontroller is utilized that can be programmed on-board through an in-circuit serial programming interface. This microcontroller is compatible with the popular Arduino development environment, and is commonly used in autonomous systems with low power and low cost requirements. Through exploiting the microcontroller's built-in 10-bit analog-to-digital converter (ADC) block as well as its computational and serial communication capability, the signals are relayed (as transduced by the sensor module and as conditioned by the analog circuitry) to the Bluetooth transceiver. It is understood that other processors/controllers, ADCs, and/or wireless protocols and transceivers can be used, and that they may be further integrated together or may be separate components.

The example conditioning path for each sensor is implemented in relation to the corresponding sensing mode. In the case of the amperometric-based glucose and lactate sensors, the originally generated signal is in the form of electrical current. Therefore, in the respective signal conditioning paths, a transimpedance amplifier stage is used to convert the signal current into voltage. In the electrical current measurements, the direction of the current is from the shared Ag/AgCl reference/counter electrode toward the working electrode of each of the glucose and lactate sensors, which would result in a negative transimpedance output voltage. Hence, for both glucose and lactate paths, the transimpedance amplifiers are followed by inverter stages to make the respective voltage signals positive, since the example ADC stage only is configured to receive positive input values (although other ADCs may be used the are configured to received negative input values, or both negative and positive input values). The feedback resistors in each of the transimpedance sections was chosen (1 MΩ for the glucose path and 0.5 MΩ for the lactate path) such that the converted voltage signal could be finely resolved, while staying within the input voltage range of the ADC stage of the microcontroller. The current sensing signal paths are capable of measuring current levels as low as 1 nA, which was significantly lower than the minimum signal in the measurements (˜10s of nA).

In this example implementation, with the transimpedance amplifier at the front-end, the Ag/AgCl reference/counter electrode of the amperometric-based sensors needed to be grounded, which prevent grounding the shared PVB reference electrodes in the potentiometric-based sensors, as the potential difference between the Ag/AgCl reference and PVB electrodes changes in the presence of different chloride ions concentrations (FIG. 6b, which shows the potential stability of a PVB coated Ag/AgCl electrode and a solid-state Ag/AgCl reference electrode (vs. commercial aqueous Ag/AgCl electrode) in different NaCl solutions), with the graph for PVB coated Ag/AgCl electrode below that of the that the Ag/AgCl reference electrode. In the case of the ISEs-based sensors, the generated signals are essentially the voltage differences between the PVB coated shared reference electrode and the working electrode of the respective sensors. Therefore, without grounding the PVB electrode the difference in potential of the floating ISE working and shared electrodes directly is measured. To this end, the signal conditioning paths of the potentiometric-based sensors included a voltage buffer interfacing the respective working and reference electrodes, followed by a differential amplifier to effectively implement an instrumentation amplifier configuration. With this approach the voltage sensing and current sensing paths are electrically isolated. Furthermore, the differential sensing stage also helped with minimizing the unwanted common-mode interferences which would have otherwise degraded the fidelity of the sensor readings. FIGS. 6c-6d illustrate the stability of a PVB coated reference electrode in solutions containing 50 mM NaCl and 10 mM of different anionic (FIG. 6c) and cationic (FIG. 6d) salts. FIG. 6a illustrates the graph of the potential v. current for the glucose sensor (with the higher peaks) and the lactate sensor.

Also, the high impedance nature of the ISE-based sensors makes advantageous the use of high impedance voltage buffers to ensure accurate open voltage measurement as intended.

The analog signal conditioning paths include a corresponding unity gain four-pole low pass filters, each with a −3 dB frequency at 1 Hz to minimize the noise and interference in the measurements. Utilizing active filters in in the system also provides flexibility in tuning the gain in the signal-conditioning path if needed or desired. The low pass filters are connected to the ADC stage of the microcontroller, to facilitate the conversion of the filtered analog signals to their respective digital forms. In an example implementation, each of the analog signal conditioning paths were electrically characterized to validate the linear output response of the channels with respect to the corresponding electrical input signals mimicking the sensor output signals. For this characterization, electrical current was applied as an input to the glucose and lactate channel terminals to model the respective amperometric-based sensor output and differential voltage was applied at the terminals of the sodium and potassium channels to model the corresponding potentiometric-based sensor output. As illustrated in FIGS. 9a-9d, all four signal-conditioning channels (Glucose, Lactate, Sodium, and Potassium) demonstrated excellent linear response (R2=1). To eliminate the non-ideal effects such as voltage offset and to obtain precise signal readings, the exact numerical linear relationship between output vs. input was obtained to map the original input signal to the analog circuit readouts, which in turn allowed for subsequent signal calibration and processing at the software level. Upon processing and averaging the data, the microcontroller relays the data to the wireless (e.g., Bluetooth) module for wireless transmission.

The example power delivery to the FISA. The FISA is powered, in this example, by a single rechargeable lithium-ion polymer battery with a nominal voltage of 3.7 V of a desired capacity (a representative 105 mAh battery is illustrated in FIGS. 9e and 9f), although other batteries and power sources may be used. The included protection circuitry protects the battery against unwanted output shorts and over-charging. Step-up DC/DC converters are used to produce a fixed, regulated output of +5 V for the microcontroller and +3.3 V for the Bluetooth modules. This regulated output also serves as the positive power supply for the analog peripheral components. The negative power supply (−5 V) for the analog peripheral components is implemented through the use of inverting charge pump DC/DC converters that produce negative regulated outputs.

The example mobile application design. A mobile application (Perspiration Analysis App) may accompany and communicate with the FISA system to provide a user-friendly interface for data display and aggregation (see, e.g., FIG. 7). In this example, In order to use certain aspects of this application, first, the user wears one or more FISA wearables and opens the Perspiration Analysis App on the mobile device. The application establishes a secure connection (e.g., a secure Bluetooth connection) to the FISA system. The FISA system transmits to the application (e.g., hosted on a mobile phone, tablet, laptop, desktop, networked television or other computing device), in real time, a stream of data (e.g. sodium, glucose, potassium, lactate, distance walked/run/cycled, time of walk/run/cycled), collected by the FISA system. The application is optionally configured to plot a graph of data values vs. time during the user's physical activities. The data and graphs can be stored on the device, uploaded to cloud servers online, and can be shared via social media. The data and graphs can be further be shared with the user's doctor(s) via the cloud, as an emailed or SMS/MMS attachment or text, or otherwise. The doctor may use such information to track the user's progress and to identify any urgent issues. Additionally, the application keeps track of the duration of exercise as well as the distance traveled. The application may be configured to operate in a various of environments and operating systems, such as Android, iOS, MacOS, Windows, Linux, Unix, etc.

FIG. 7a illustrates the application homepage after Bluetooth pairing of the wearable with a mobile device. FIG. 7b illustrates a real-time data display of sweat analyte levels as well as the skin temperature during exercise, and shows the distance walked/run/cycled, and the exercise time. FIG. 7c illustrates the real-time data progression/trends of an individual sensor. FIG. 7d illustrates available data sharing and uploading options (e.g., upload to an cloud drive, sharing via a social network, sharing with a local device, sharing via text/multimedia messaging, sharing via email, etc.). The application can similarly be used to display information and generate alerts regarding heavy metals, Ca²⁺ and/or pH collected via the wearable as discussed elsewhere herein. For example, the application may be configured to detect, using thresholds or other technique, when a user's readings fall outside to a normal or safe range and generate an alert notifying the user and/or other entities of the event. The application may also identify in the alert a name for the user's condition or a risk of a condition occurring based at least in part on the sensor readings (e.g., hyponatremia, hypokalemia, muscle cramps or dehydration, pressure ischemia, panic disorder, Frey's syndrome, pressure ulcers, Wilson's disease, heart and kidney failure, liver damage, brain disease and disorder, anemia, osteoporosis, respiratory problems, liver problems, kidney problems, Hunter-Russell syndrome, Minamata disease, acrodynia, myeloma, acid-base balance disorder, cirrhosis, renal failure, normocalcaemic hyperparathyroidism, hyperparathyroidism, kidney stones, other conditions discussed herein, and the like).

The characterization of the sensors. A set of example electrochemical sensors was characterized to explore their reproducibility in solutions of target analytes. FIGS. 10a-10d (which illustrate the reproducibility of the sodium (FIG. 10a), potassium (FIG. 10b), glucose (FIG. 10c) and lactate (FIG. 10d) sensors (8 samples for each kind of sensor)) show that Na⁺ and K⁺ sensors had relative standard deviation (R.S.D.) of ˜1% in sensitivity while glucose and lactate sensors had R.S.D. of ˜5% in sensitivity. However, there are differences in absolute potentials values for ISEs in the same solution. Therefore, one-point calibration in a standard solution containing 1 mM KCl and 10 mM NaCl was performed for Na⁺ and K⁺ sensors prior to each use. The measured potential of ISEs at the standard solution was then set as zero by the microcontroller. Such calibration is similar to what is done in the commercial finger-stick glucose sensors. No calibration was needed for the glucose and lactate sensors. Long-term stability of the sensors was also evaluated over a period of 4 weeks using 5 different sensor arrays each week (FIGS. 10e-h, where the error bars represent the standard deviations of the measured data for 5 samples.). It was observed that the Na⁺ and K⁺ sensors had approximately the same sensitivities of 62.5 and 59.5 mV per decade of concentration (mV/dec), respectively, in ambient conditions. The sensitivities of the glucose and lactate sensors similarly were maintained within 5% of their original values over the 4 weeks period when stored at 4° C. The glucose and lactate sensors were characterized chronoamperometrically using a Gamry Electrochemical Potentiostat (FIGS. 2a and 2b). Due to Faraday and capacitive currents 38, the responses of both sensors showed drift initially but stabilized within 1 minute of the data recording. The in vitro temperature compensation experiments (FIG. 2h) were performed continuously using the same sensor in 4 petri dishes containing solutions at different temperatures on different hot plates. The convection and non-uniform distributions of solution temperature could result in noticeable noise in the signal measurements.

For continuous use, all the sensors displayed excellent stability over the entire exercise period. The sensor array could be repeatedly used for continuous temperature and sweat electrolyte monitoring. However, the glucose and lactate responses degraded beyond the exercise period (after two hours) due to decreased enzyme activity. The devised sensor-FPCB interface allows for convenient replacement of the fresh sensor arrays for subsequent use.

Analysis of the effect of mechanical deformation on the sensors was performed by repeatedly bending Na⁺, glucose sensors, and temperature sensors (radius of curvature, 1.5 cm) as well as FPCB (radius of curvature, 3 cm) for a total of 60 cycles (FIG. 12). Performance of the sensors was recorded after every 30 cycles. Continuous measurement on sensor performance while bending and no bending was also performed.

Ex-situ evaluation of the sweat samples. Ex-situ sensor performance was also conducted by testing subjects' sweat samples collected from their forehead. Sweat samples were collected every 2 to 4 minute by scratching their foreheads with microtubes, and subjects' foreheads were wiped and cleaned with gauze after every sweat collection 19. The changes of [Na⁺] and [K⁺] during euhydration and dehydration trials were also studied ex-situ in the same manner. The calibration of the sensor arrays was performed prior to ex-situ measurements using artificial sweat containing 22 mM urea, 5.5 mM lactic acid, 3 mM NH⁴, 0.4 mM Ca²⁺, 50 μM Mg²⁺, and 25 μM uric acid with varying [glucose] from 0 to 200 μM, [K⁺] from 1 to 16 mM and [Na^{+] from} 10 to 160 mM.

The example setup of FISA for on-body testing. A water absorbent thin rayon pad was placed between the skin and the sensor array during on-body experiments to absorb and maintain sufficient sweat for stable and reliable sensor readings, and to prevent direct mechanical contact between the sensors and skin. The pad could only absorb ˜10 μL sweat which was sufficient to provide stable sensor readings. During on-body tests, the newly generated sweat would refill the pad and ‘rinse away’ the old sweat. The on-body measurement results were also consistent with ex-situ tests using freshly collected sweat samples. The refill time was estimated to be less than 1 minute based on the sweat rate (˜3-4 mg/min/cm2) and the pad size (1.5 cm×2 cm). The intrinsic response time of FISA was smaller than body's response time to the changes in physiological conditions. An increase in temperature was observed when the smart headband or smart wristband was worn due to the use of the plastic substrate on skin. While this may result in a small error in measuring the actual skin temperature, it should be noted that this does not have an impact on the measurement of the electrolytes and metabolites due to the on board temperature calibration. To further ensure fidelity of sensor readings, the data collection of each channel took place when sufficient sweat sample was present, as evident by stabilization of sensor readings (within 10% variations between the continuous 5 data points) within the physiologically relevant range ([Na⁺]: 20-120 mM, [K⁺]: 2-16 mM, [glucose]: 0-200 μM, [lactate]: 2-30 mM).

Example on-body sweat analysis. The example on-body evaluation of the FISA was performed in compliance with the protocol that was approved by the institutional review board (IRB) at the University of California, Berkeley (CPHS 2014-08-6636). Twenty six healthy subjects (4 females and 22 males), aged 20-40, were recruited. The study was conducted as three trials: constant workload cycle ergometry, graded workload cycle ergometry, and outdoor running. Constant workload cycle ergometry was conducted on 14 volunteers (4 females and 10 males between the ages of 20 and 40). The graded cycle ergometry was conducted on 7 male volunteers (who were also involved in the constant workload cycle study). 12 male volunteers between the ages of 20 and 40 were recruited for outdoor running study. An electronically braked leg-cycle ergometer (Monark Ergomedic 839E, Monark Exercise AB, Vansbro, Switzerland) was used for cycling trials which included real-time monitoring of heart rate (HR), oxygen consumption (VO₂), and pulmonary minute ventilation (VE). The power output (PO) was calibrated and monitored through the ergometer. HR was measured using a Tickr heart rate monitor (Wahoo fitness), and VO₂ and VE were continuously recorded throughout trials via an open-circuit, automated, indirect calorimetry system (TrueOne metabolic system; ParvoMedics, Sandy, Utah).

The FISAs were packaged in traditional sweatbands during the indoor and outdoor trials. The sensor arrays were calibrated, and the subjects' foreheads and wrists were cleaned with alcohol swabs and gauze before sensors were worn on body. For the constant workload cycling trial subjects were cycling at 50 W with 50 W increments every 90 s to 150 W, and 20 minutes of cycling at 150 W. The PO was then decreased by 50 W every 90 s. The graded workload trial consisted of 5 minutes of seated rest followed by cycling at 75 W for 20 minutes and then cycling at 200 W until fatigue followed by a 10 minutes rest. The outdoor running trial was conducted with a group of 12 subjects in which six were instructed to drink 150 mL water every 5 minutes and six did not drink water throughout the trial. Subjects consented to run until volitional fatigue at a self-selected pace (˜12 km/h) and the Na⁺ and K⁺ sensors responses (from their foreheads) were recorded.

SUPPLEMENTARY TABLE 1
EXAMPLES OF WEARABLE SWEAT BIOSENSORS.

| | | | | Re- |
|---|---|---|---|---|
| | Recognition | | | fer- |
| Platform | element | Methods | Analyte | ence |
| Textiles | Bromocresol | Colorimetry | pH | 1 |
| | purple (BCP) | | | |
| Cotton | Bare carbon | Amperometry | β- | 2 |
| | | | nicotinamide | |
| | | | adenine | |
| | | | dinucleotide | |
| | | | and | |
| | | | hydrogen | |
| | | | peroxide | |
| Polyimide/Lycra | Sodium | Potentiometry | Sodium | 3 |
| blend | ionophore | | | |
| Textiles, | Sodium , | Potentiometry | Sodium, pH | 4 |
| | ionophore | | | |
| Polyimide/Lycra | bromocresol | Colorimetry | | |
| blend | purple | | | |
| | (BCP), | | | |
| Parylene | Lactate oxidase | Transistor based | Lactate | 5 |
| | | conductometry | | |
| (Poly(methyl- | Bromocresol | Colorimetry | pH | 6 |
| methacrylate) | purple (BCP) | | | |
| Cotton yarns | Nonactin, | Potentiometry | Ammonium, | 7 |
| | valinomycin, and | | potassium | |
| | tridodecylamine | | and pH | |
| Polyester | Ag/AgCl | Potentiometry | Chloride | 8 |
| Elastomeric | Carbon | Voltammetry | Uric acid | 9 |
| stamps | | | | |
| Temporary tattoo | Lactate oxidase | Amperometry | Lactate | 10 |
| Temporary tattoo | Polyaniline | Potentiometry | pH | 11 |
| Temporary tattoo | Ammonium | Potentiometry | Ammonium | 12 |
| | ionophore | | | |
| Temporary tattoo | Sodium | Potentiometry | Sodium | 13 |
| | ionophore | | | |
| Temporary tattoo | Bismuth | Stripping | Zinc | 14 |
| | | voltammetry | | |
| Polyimide | Sodium | Potentiometry | Sodium | 15 |
| adhesive patch | ionophore | | | |

### Ca²⁺and pH

The wearable sensing platform described herein can be configured with additional or different features. As discussed herein, optionally the wearable sensing platform can simultaneously and selectively measure detailed profiles of Ca²⁺ and pH in real-time through a fully integrated wearable sensing system that can be worn during the course of normal daily activities.

Homeostasis of ionized calcium in biofluids is critical for human biological functions and organ systems. However, conventionally measurement of ionized calcium for clinical applications is not easily accessible due to its strict procedures and dependence on pH. Further, pH balance in body fluids greatly affects metabolic reactions and biological transport systems. A wearable electrochemical device is disclosed for monitoring (e.g., continuous monitoring) of ionized calcium and pH of body fluids using an array of Ca²⁺ and pH sensors (e.g., a disposable and flexible array of Ca²⁺ and pH sensors) that interfaces with a printed circuit board (e.g., flexible printed circuit board).

The disclosed platform enables real-time quantitative analysis of these sensing elements in body fluids such as sweat, urine, and tears. Accuracy of Ca²⁺ concentration and pH measured by the wearable sensors is validated through inductively coupled plasma-mass spectrometry technique and a commercial pH meter, respectively. Test results show that the wearable sensors have high repeatability and selectivity to the target ions. Real-time on-body assessment of sweat is also performed, and test results indicate that calcium concentration increases with decreasing pH. The disclosed platform can optionally be used in noninvasive continuous analysis of ionized calcium and pH in body fluids for disease diagnosis such as primary hyperparathyroidism and kidney stones.

Calcium is an essential component for human metabolism and minerals homeostasis. Indeed, about 1%-2% of human body weight is made up of calcium. Excessive alternation of ionized calcium levels in biofluids can have detrimental effects on the function and structure of many organs and systems in the human body, including myeloma, acid-base balance disorder, cirrhosis, renal failure, and normocalcaemic hyperparathyroidism. Free Ca²⁺ is conventionally measured in body fluids, such as urine for estimating kidney stone-forming salts. A person's pH can be another significant component for potential disease diagnosis.

For example, kidney stone patients with type II diabetes are reported to have a lower pH than normal individuals. Change in pH of skin, which is due to sweat, has been reported to take part in the development of skin disorders such as dermatitis, ichthyosis, and fungal infections. Additionally, free Ca²⁺ level in biofluids is dependent on pH. Therefore, rigorous processes and rapid analysis of Ca²⁺ with pH correction are conventionally performed in special laboratories within hours of samples extraction for accurate analysis of biofluids. Such applications can become easier by in situ measurement of Ca²⁺ and pH in body fluids through an in-depth data analysis performed using a reliable wearable sensing platform, such as that disclosed herein. However, conventionally, wearable Ca²⁺ sensors for real-time health assessment via body fluids has not been performed. On the other hand, careful analysis of the pH of body fluids is needed for more accurate in situ measurement. The use of flexible electronics and Ca²⁺ sensors having conformal contact with the human body, as disclosed herein, provides a more accurate and reliable epidermal quantitative analysis.

The disclosed integrated wearable sensing system performs real-time multiplexed sensing of human perspiration which enables accurate measurement of sweat analytes through signal processing and calibration. Considering the importance of Ca²⁺ and pH and their relationship in body fluids, it is desirable to simultaneously and selectively measure detailed profiles of Ca²⁺ and pH through an integrated wearable sensing platform during the course of normal daily activities with real-time feedback.

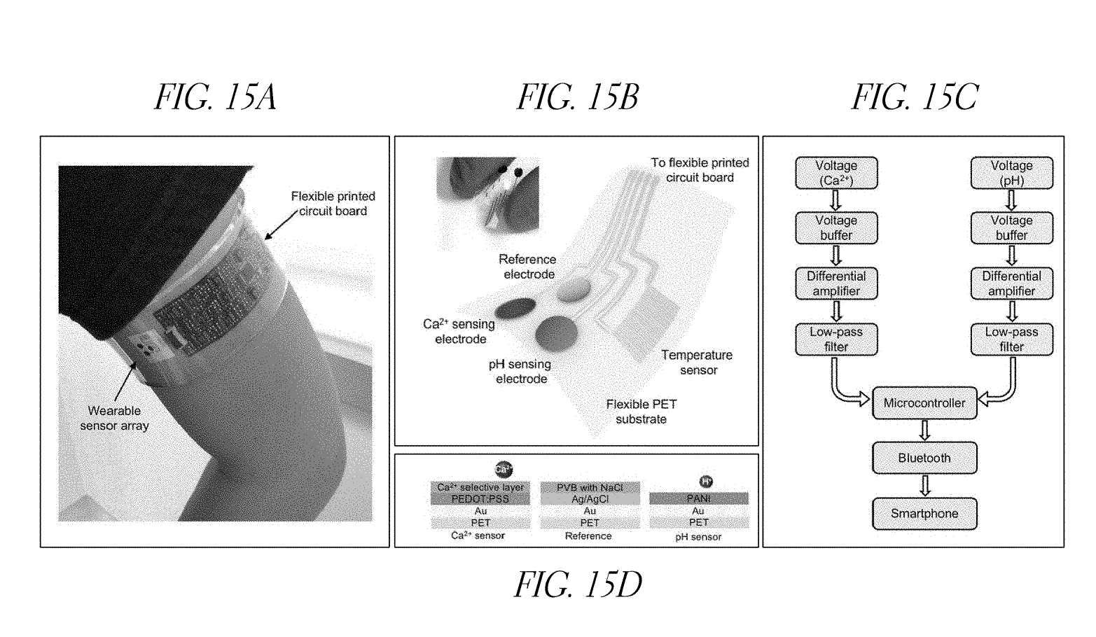

The disclosed wearable sensing system is configured to monitor in real-time Ca²⁺ concentration and pH of body fluids as well as skin temperature (see, e.g., FIG. 15a). The disclosed system provides accurate determination of Ca²⁺ and pH in body fluids including sweat, urine, and/or tears. The disclosed system determines free Ca²⁺ concentration and pH by direct measurement of body fluids, such as sweat generated during cycling, walking or running. Such immediate analysis after fluid secretion reduces or minimizes cross-contamination and avoids delayed sample analysis, while be very convenient. These features also facilitate a closer examination of real-time change in Ca²⁺ concentration with pH, and reduces the need for pH correction in clinical diagnosis such as hypercalcemia and hypocalcemia tests.

Certain figures will now be summarized.

FIG. 15a illustrates an integrated wearable multiplexed sensing system on a subject's arm. FIG. 15b depicts a schematic of a flexible sensor array containing Ca²⁺, pH, and temperature sensors patterned on a flexible PET substrate. The inset depicts a flexible sensor array. FIG. 15c depicts surface membrane compositions of a Ca²⁺, a reference, and pH sensing electrodes. FIG. 15d depicts a schematic of a FPCB system for signal conditioning of Ca²⁺ and pH sensors, data analysis via a processor (e.g., microcontroller, and data transmission to a mobile device wirelessly (e.g., a mobile phone) via a Bluetooth transceiver).

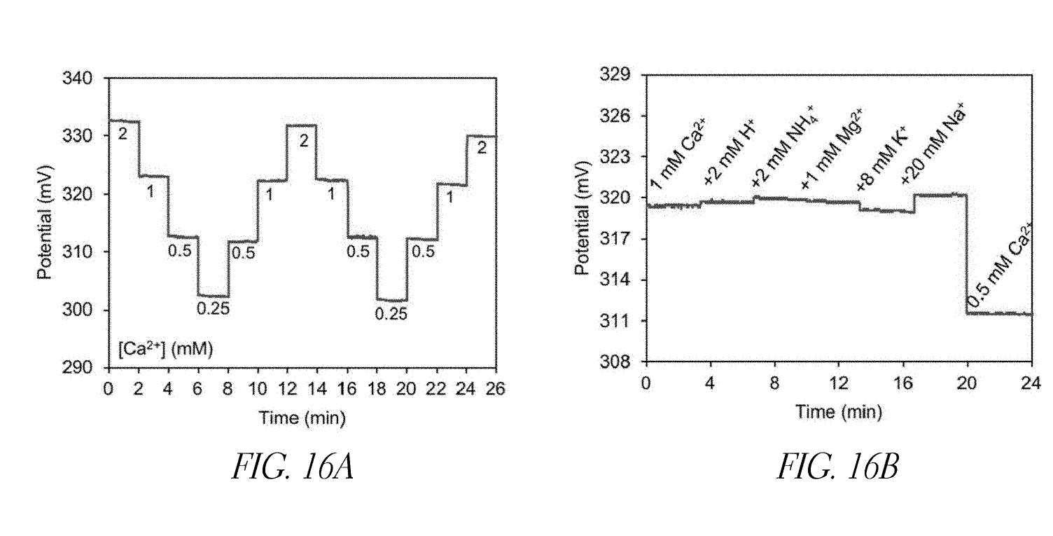

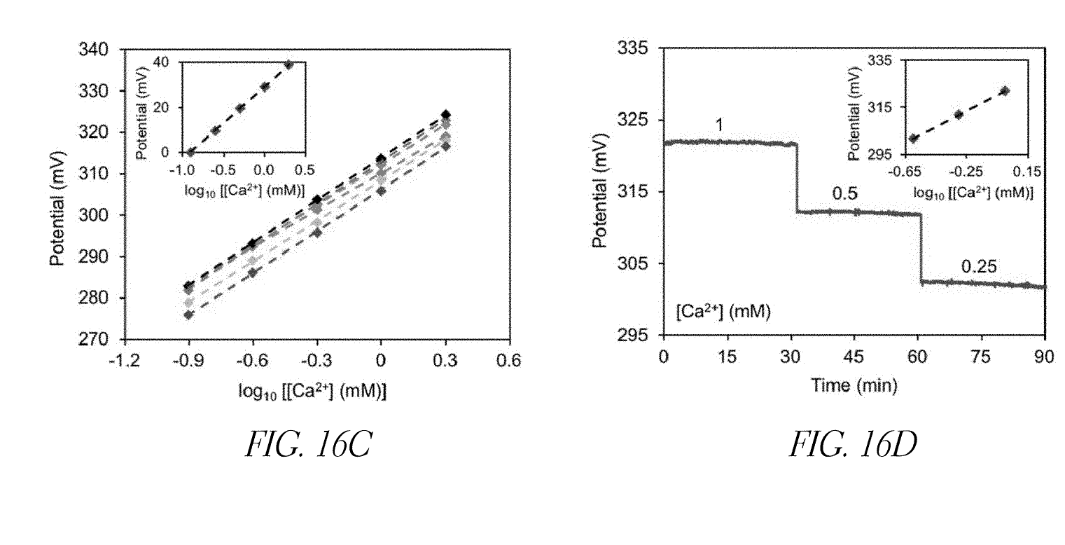

FIG. 16 illustrates aspects Ca²⁺ sensor performance. FIG. 16a illustrates sensitivity and repeatability. FIG. 16b illustrates selectivity. FIG. 16c illustrates reproducibility of Ca²⁺ sensors (n=6). FIG. 16d illustrates long-term stability in 0.01 M acetate buffer of pH 4.6. The inset in FIG. 16c shows an average linear relationship of n=6 between open circuit potential and [Ca²⁺] in logarithmic scale. Potentials at 0.25 mM [Ca²⁺] are set to zero. The inset in FIG. 16d indicates a linear relationship between open circuit potential and [Ca²⁺] in logarithmic scale.

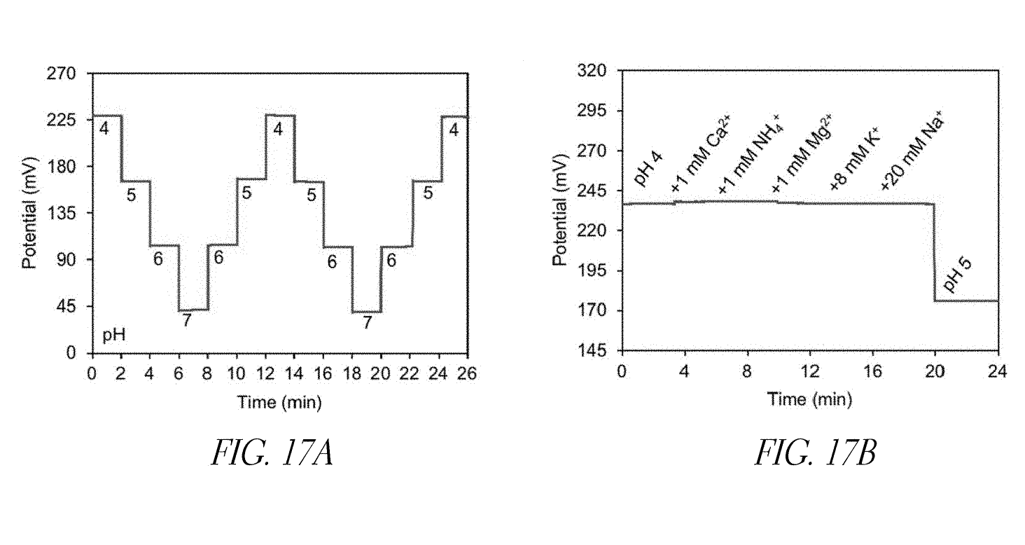

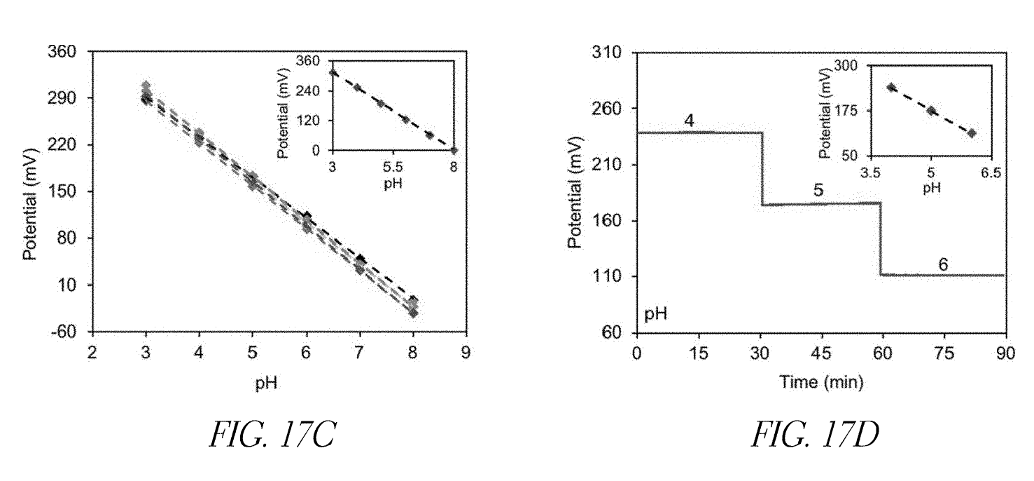

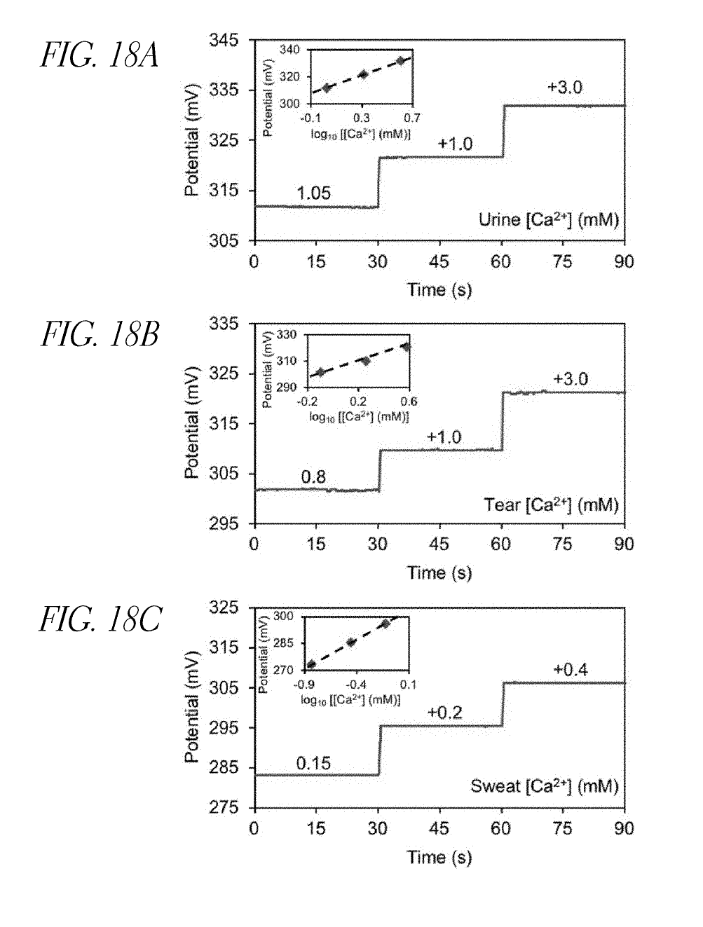

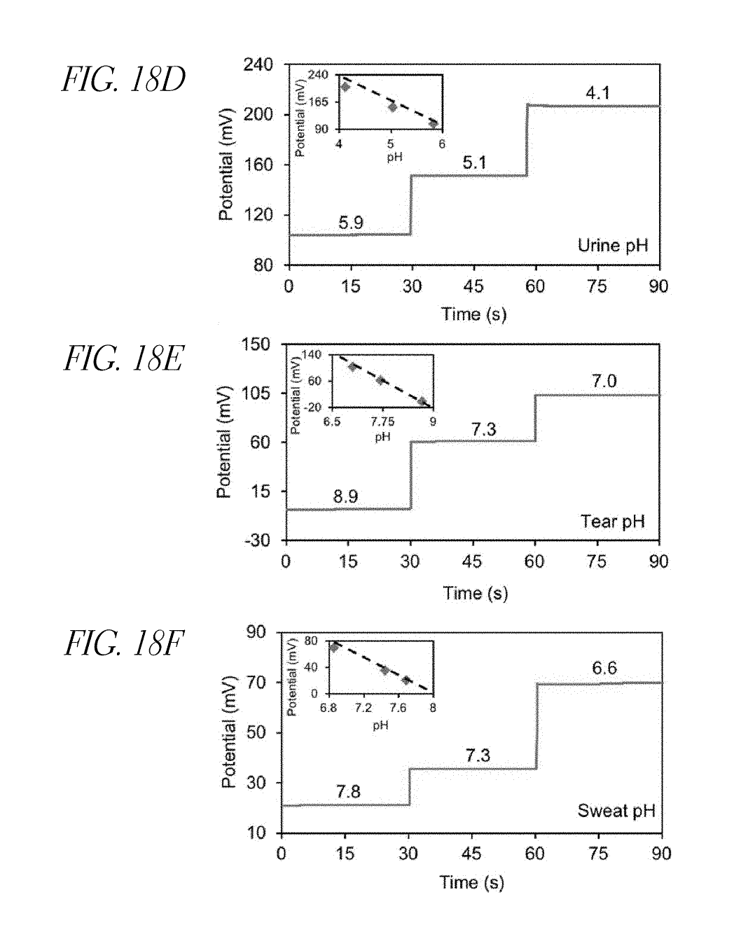

FIG. 17 illustrates aspects of pH sensor performance. FIG. 17a illustrates sensitivity and repeatability. FIG. 17b illustrates selectivity. FIG. 17c illustrates reproducibility of pH sensors (n=6). FIG. 17d illustrates long-term stability in Mcllvaine's buffer. The inset in FIG. 17c shows an average linear relationship of n=6 between open circuit potential and pH. Potentials at pH 8 are set to zero. The inset in FIG. 17d indicates a linear relationship between open circuit potential and pH.

FIG. 18 illustrates off-body evaluations of Ca²⁺ (FIGS. 18a-18c) and pH (FIGS. 18d-18f) sensors in urine (FIG. 18a, d), tear (FIG. 18b, e), and sweat (FIG. 18c, f). Measurements of [Ca²⁺] are performed by consecutively adding Ca²⁺ into raw biofluids. The amounts added are indicated in the figures. Measurements of pH are performed by consecutively adding HCl into raw biofluids. The final pH is determined using a conventional pH meter. The linear regression lines in inset figures correspond to sensors' responses in standard calibration solutions obtained in FIG. 16c for Ca²⁺ measurement and in FIG. 17c for pH measurement.

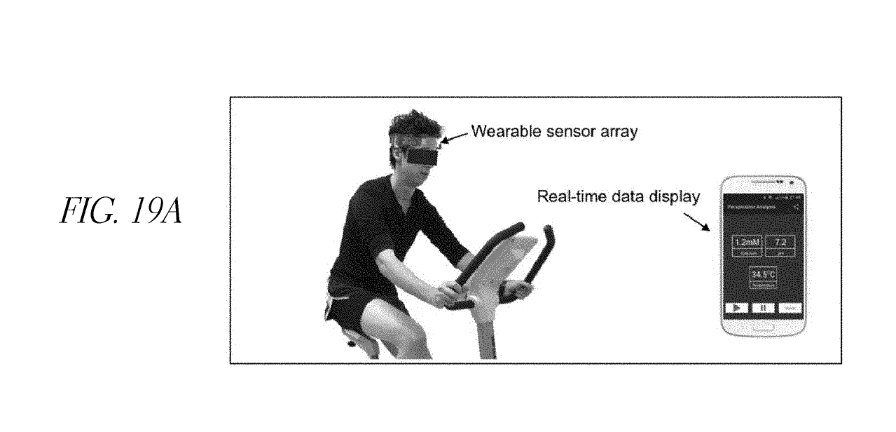

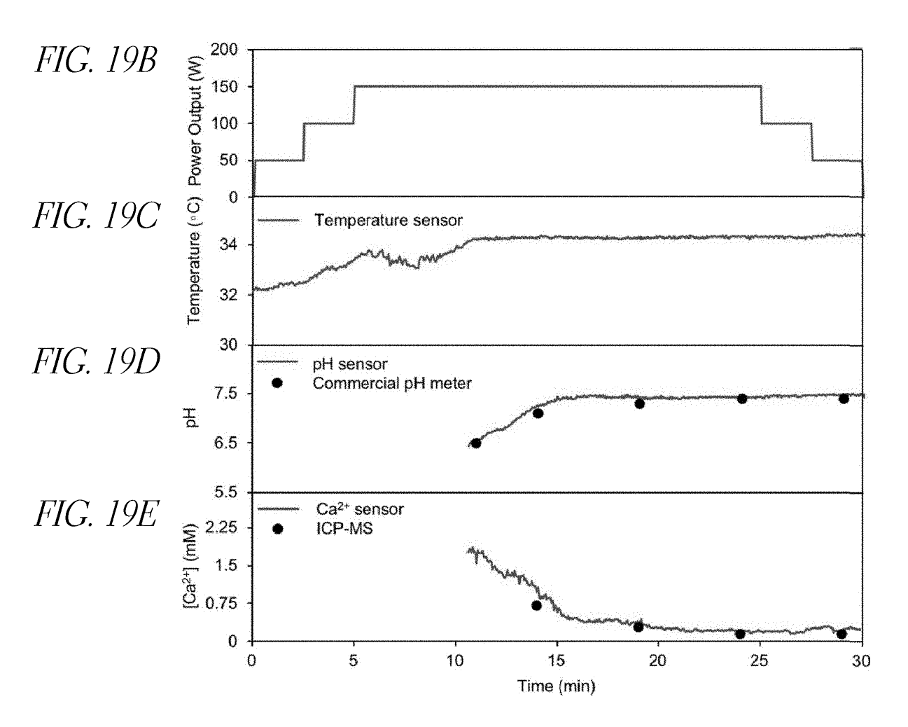

FIG. 19 illustrates real-time on-body analysis of human perspiration during a constant-load cycling. FIG. 19a depicts a wearable multiplexed sensing system worn on a subject's forehead during stationary cycling. Data from the wearable system is transmitted to an application hosted on a user device (e.g., a mobile device, such as a mobile phone, or other computing device) and stored on the user device. FIG. 19b depicts a subject's cycling power output v. time and real-time sweat analysis results. FIG. 19c depicts measures skin temperature v. time. FIG. 19d depicts measured pH v time. FIG. 19e depicts Ca²⁺ concentration measured using the wearable sensing system. Black dots in FIGS. 19d and 19e correspond to measurements performed by a pH meter and by ICP-MS, respectively.

The example wearable sensing system includes an electrochemical platform comprising a Ca²⁺ sensor, a pH sensor, and/or a skin temperature sensor. The sensors may be plastic-based biosensors that are fabricated on a flexible polyethylene terephthalate (PET) substrate by common physical evaporation and electrochemical deposition methods as illustrated in FIG. 15b. Measurements of Ca²⁺ concentrations and pH are based on ion-selective electrodes (ISEs), coupled with a polyvinyl butyral (PVB)-coated Ag/AgCl reference electrode (RE). Electrical potential differences between the ISEs and a RE, proportional to the logarithmic concentration of respective target ions, are measured with the aid of the interfacing signal conditioning circuitry. The Ca²⁺ sensing electrode comprises a thin organic membrane containing electrically neutral carrier calcium ionophore II (ETH 129) and an ion-electron transducer (PEDOT:PSS), and the pH sensing electrode detects H+ by deprotonation at the surface of polyaniline (PANI). The RE is coated with a PVB layer containing saturated NaCl to achieve a stable potential regardless of ionic strengths of test solutions.

Surface membrane compositions of the electro-chemical electrodes are demonstrated in FIG. 15c. The resistive temperature sensor is optionally based on Cr/Au microlines. The flexible sensor array interfaces with a circuit board, such as a flexible printed circuit board (FPCB) that includes signal transduction, conditioning, processing, and wireless transmission. As depicted in FIG. 15d, a differential amplifier is used to measure the voltage output of the Ca²⁺ and pH sensors, which corresponds to the voltage difference between the PVB-coated shared RE and the ISEs. The high impedance of the ISE-based sensors, coupled to a high-impedance voltage buffers in front of the differential amplifier, ensures accurate open circuit voltage measurement. The signal is then passed to a low-pass filter to filter high frequency noise and electromagnetic interferences. The corresponding filtered signals are digitized via an ADC, and read and processed further by a processing device (e.g., a microcontroller). The data is then wirelessly transmitted (e.g., via Bluetooth or WiFi) to remote device (e.g., a mobile device, such as a cell phone, tablet computer, laptop, etc., or a non-mobile device, such as a desktop computer or large screen networked television) and received and displayed via an application hosted on the remote device.

### Results And Discussion With Respect To pH and Ca²⁺

Concentration of Ca²⁺ in human body fluids commonly varies from 0.5 to 3 mM. Due to the limited Ca²⁺ concentration range, sensitivity is important to ensure accurate measurements. ETH 129 is utilized as the Ca²⁺-selective ionophore due to its ability to translocate Ca²⁺ across biological membranes. FIG. 16a illustrates performance aspects of a Ca²⁺ sensor in 0.01 M acetate buffer solutions containing 0.25-2 mM Ca²⁺.

Dynamic response of the Ca²⁺ sensor under consecutive change from high to low and then to high Ca²⁺ concentrations is performed two times. Since Ca²⁺ is a divalent ion, the ideal sensitivity of an electrochemical Ca²⁺ sensor at standard temperature is 29.6 mV/decade of ion concentration, which is half of a monovalent ion, based upon the Nernst equation. The Ca²⁺ sensor shows a near-Nernstian response with an average of 32.7 mV/decade in two complete cycles. The senor shows fast response to changes in Ca²⁺ level with a 3.0% relative standard deviation (RSD) of sensitivity. This indicates that Ca²⁺ detection by the sensor is reproducible and durable under repetitive testing.

Body fluids contain a variety of electrolytes such as Ca²⁺, Mg²⁺, Na+, K⁺, H⁺, and NH4⁺. One desirable aspect of a wearable electrochemical sensor is its ability to selectively discriminate and measure target ions. Thus, the influence of these major electrolytes on sensor's performance is examined. In this study, interfering ions with physiological relevant concentrations (2 mM H⁺, 2 mM NH4⁺, 1 mM Mg²⁺, 8 mM K⁺, and 20 mM Na⁺) are subsequently added into 1 mM Ca²⁺ solution, and measurements are performed after 20 seconds waiting time. The change in potential due to addition of such ions, as demonstrated in FIG. 16b, is significantly smaller than the response for typical physiological [Ca²⁺] variations (e.g., 1 mM to 0.5 mM). This demonstrates that the sensor is selectively responsive to Ca²⁺ and hence makes it feasible for body fluid analysis.

Additionally, it is beneficial for sensors to be reproducible such that reliable analysis can be attained from individual sensors. Six sample sensors were tested in a solution containing 0.125-2 mM of Ca²⁺ concentration range. As displayed in FIG. 16c, the absolute potentials of these six sensors range from 275.9 to 283.0 mV in the presence of 0.125 mM Ca²⁺.

These sensors show sensitivity ranging from 29.8 to 34.2 mV/decade of concentration, with an average sensitivity of 32.2 mV/decade and a RSD of 1.5%. The value of average sensitivity is used as a standard slope for calibration in later studies of biofluids. The variations in the absolute potentials of different sensors (resulted from sensor preparations and manually dropcasting method) are resolved by one-point calibration, as shown in inset of FIG. 16c, in which the open-circuit potential of the sensors in 0.125 mM Ca²⁺ is set to zero by the processor (e.g., microcontroller).

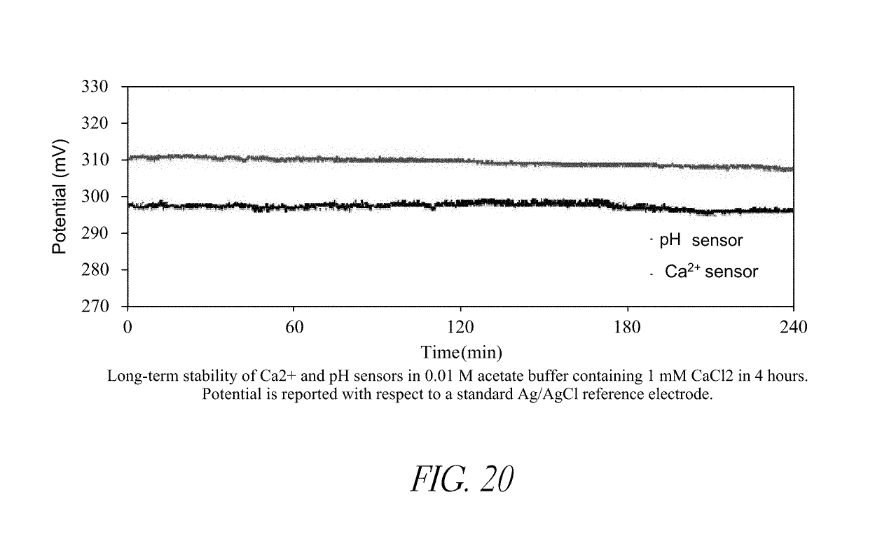

This is similar to commercial pH meters where a standard solution is used to calibrate the measurement before actual measurement is undertaken. In the case of long-term analysis on Ca²⁺ concentration in body fluids, variation due to potential drift can easily conceal the actual measurement results. To test this, the Ca²⁺-selective sensor is kept under 0.25-1 mM Ca²⁺ solutions for a total of 90 minute and under 1 mM solution for 4 hours, as presented in FIGS. 16d and 20, respectively. A sensitivity of 33.7 mV/decade is measured (see, FIG. 16d), and a potential drift of 1.1 mV/h is observed in FIG. 20. These results reveal that the Ca²⁺ sensors can yield a small error of approximately 8% over an hour of continuous measurement without large deviation from the average sensitivity.

Similar to the Ca²⁺ sensors, aspects of the performance of the pH sensors are evaluated. PANI is one medium for pH measurement in body fluids due to its ease of fabrication, reproducibility, and biocompatibility. In this study, H⁺-selective PANI film is electrochemically deposited onto a Au electrode by cyclic voltammetry. The resulting PANI-based pH sensor presented in FIG. 17a was tested repeatedly in McMaine's buffer from pH 4 to 7. Results exhibited an average slope of 62.5 mV/decade with RSD 1.0% in two complete cycles from pH 4 to 7 and then to 4 with one-unit increments.

The pH sensor is also selective to H+ with a potential variation of approximately 3.1% compared to its sensitivity as shown in FIG. 17b. Since pH in human body fluids commonly fluctuates between 3 and 8 depending on specific fluids, sensors are characterized from pH 3 to 8. Reproducibility of six sensors is reported in FIG. 17c. Results show that absolute potentials range from 285.6 to 309.8 mV at pH 3 and sensitivities vary from 60.0 to 65.4 mV/decade of concentration. Regardless of the variation in absolute potentials, these sensors show only a RSD of 2.3% in sensitivity with a 63.3 mV/decade average. This average sensitivity is later used as a standard calibration value for measurement in body fluids. Long-term performance of the pH sensor is captured in FIGS. 17d and 20. The results in FIG. 17d show that sensitivity of pH sensor is 63.7 mV/decade over a 90 minute measurement. FIG. 20 indicates that deviation due to potential drift is 0.7 mV/h which corresponds to 1.1% error in pH value over an hour of continuous measurement.

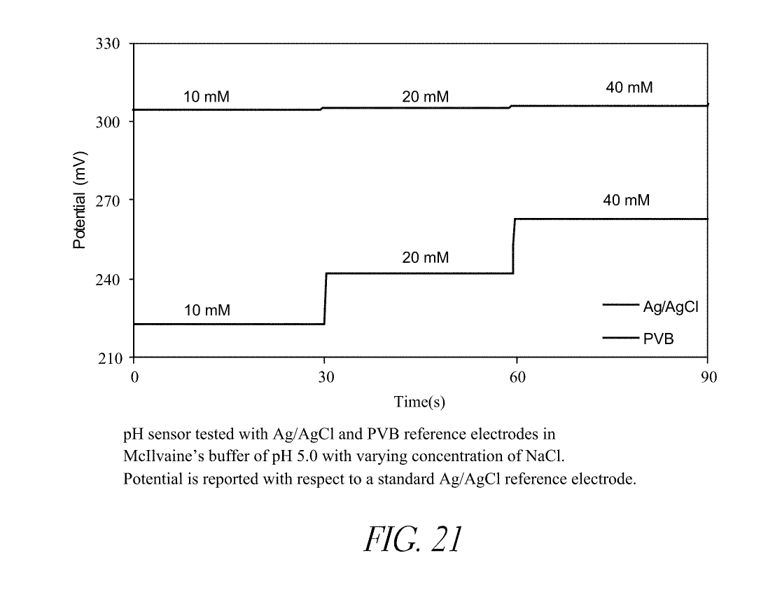

Conventional wearable pH sensors are not sufficiently accurate for detailed quantitative analysis in body fluids due to Cl− influence on solid-state Ag/AgCl RE. Here, pH sensing with Ag/AgCl and PVB-based Ag/AgCl REs is compared under a constant pH 5.0 with varying Cl− concentrations. FIG. 21 depicts that variation of Cl− concentration greatly influences the performance of the sensor with Ag/AgCl RE (shown toward the bottom of the chart), while that with PVB-based RE (shown toward the top of the chart) remains relatively stable. This is because the PVB layer contains Cl− and is immune to change in Cl− concentrations. The above results exhibit that present pH sensors provide an accurate and reliable analysis capability compared to previously reported electrochemical pH sensors.

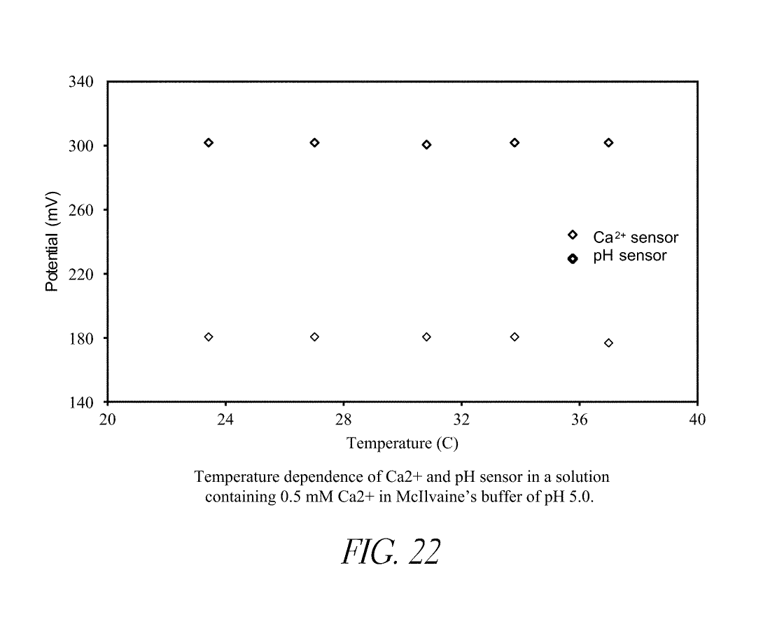

Skin temperature is an effective marker of the thermal state of individuals and is also informative for many skin related diseases (such as ulceration). Performance aspects of Cr/Au-based temperature sensors are discussed above. Such resistive temperature sensors have a sensitivity of 0.18% per ° C. with respect to its baseline resistance at room temperature, although other temperatures sensors with different sensitivity may be used. To investigate the influence of temperature on Ca²⁺ and pH sensors, sensors are tested in temperatures ranging from 23 to 37° C. in McIlvaine's buffer of pH 5.0 containing 0.5 mM Ca²⁺. Unlike enzymatic sensors, in which the performance is greatly influenced by the change in temperature, both Ca²⁺ and pH sensors show no significant response to temperature change as illustrated in FIG. 22. In order to better ensure accurate measurement of body fluids, Ca²⁺ and pH analyses of human sweat and urine samples using the wearable sensors are validated with inductively coupled plasmamass spectrometry (ICP-MS) technique and a commercial pH meter, respectively. Measurements of biofluids using sensors are computed based on a calibration curve obtained from artificial body fluids (detailed in the Experimental Section). Results displayed in Table 2 show Ca²⁺ concentrations and pH in sweat and urine measured by ICP-MS and Ca²⁺-selective sensors and by commercial pH meter and pH sensors.

In this example, measurements of sweat and urine [Ca²⁺] acquired by sensors vary by a maximum of 7.0% and 10%, respectively, from the ICP-MS results. On the other hand, in this example pH sensors show <2.2% and 3.6% variations in sweat and urine from a commercial pH meter. These variations are relatively small, compared to normal range of [Ca²⁺] and pH of body fluids.

To further confirm the accuracy of sensor readings, additional studies were made by adding fixed amounts of Ca²⁺ and H+ into raw sweat, urine, and tear samples, and the change in potential with concentration was examined. FIGS. 18a-c presents a change in potential upon addition of Ca²⁺ in urine, tear, and sweat, respectively. These measurements lie along a standard calibration line obtained in FIG. 16c with a slope of 32.2 mV/decade. Such a method provides an alternate mean to verify the sensors' measurement accuracy. Due to the buffering capacity of body fluids, the actual pH of the solutions is measured with a commercial pH meter after every H+ addition to raw urine, tears, and sweat samples. Measured pH results in urine, tears, and sweat are plotted against the potentials in FIG. 18d-f. Similar to the Ca²⁺ sensors, measurements of pH sensors lie nearly along the calibration line with a slope of 63.3 mV/decade. The study confirms that complex body fluids have relatively minimal interference with sensors' readings.

TABLE 2

| | [Ca²⁺] (mM) | | pH | |
|---|---|---|---|---|
| Bodily Fluid | Ca²⁺ sensor | ICP-MS | pH sensor | pH meter |
| sweat 1 | 0.96 | 0.92 | 4.4 | 4.5 |
| sweat 2 | 0.48 | 0.48 | 7.2 | 7.3 |
| sweat 2 | 0.76 | 0.71 | 8.1 | 8.1 |
| urine 1 | 1.8 | 1.8 | 7.6 | 7.8 |
| urine 2 | 7.2 | 7.1 | 6.3 | 6.5 |
| urine 3 | 1.2 | 1.1 | 6.4 | 6.6 |

Following the ex situ analysis of sweat, urine, and tear, real-time on-body evaluation in human perspiration using the flexible integrated wearable device was also performed. As illustrated in FIG. 19a, in this example, a subject wears a headband embedded with the fully integrated flexible sensing system while cycling. Real-time analysis is then wirelessly transmitted to a device (e.g., a mobile phone or other device) and displayed in an application hosted by the device. On-body assessment of sweat Ca²⁺ and pH was performed with a 5 minute ramp-up and a 20 minute biking at a power of 150 W, followed by a 5 minute cool-down session (FIG. 19b). Sweat is simultaneously collected for analysis using ICP-MS and a commercial pH meter. FIG. 19c shows change in skin temperature with exercise time. Initially, the temperature increases as exercise progresses, and a trough in the measurement curve is observed between 6 and 11 minute of cycling time. This indicates that perspiration begins and initiates the measurements of other ion-selective sensors.

In this example, temperature then remains stable in the rest of the cycling time, as similarly discussed above. FIG. 19d depicts real-time sweat pH profile with exercise time. Initially, the sensors have no response during the first 10 minute because there is not enough sweat generated. After 10 minute into cycling, sweat pH is observed to increase gradually for 5 minute which is mainly due to a decrease of lactic acid concentration in sweat.32 Sweat pH then stabilizes in the remaining 15 minute of exercise. This on-body result had close readings with a commercial pH meter. In FIG. 19e, the Ca²⁺ sensor shows an opposite trend compared to pH. Concentration of Ca²⁺ initially decreases rapidly with increasing pH and stabilizes after 15 min. ICP-MS result also shows a similar trend with slightly lower concentrations than the on-body readings. This result is consistent with literature which reports an inverse relation between concentration of Ca²⁺ and pH.

These on-body results further affirm the utilization of the wearable system in personal health care. Such real-time continuous analysis can alert the wearer regarding excessive loss or rise of electrolytes.

Thus, a fully integrated wearable electrochemical platform for simultaneous in situ analysis of Ca²⁺ and pH in body fluids is disclosed. The wearable system, containing flexible sensors coupled with integrated circuits and a wireless transceiver, enables accurate measurements of characteristics of biofluids, including urine, tear, and sweat with real-time feedback. The disclosed wearable sensing systems offers many advantages over the traditional extensive laboratory analysis for accurate measurement of analytes in complex biofluids. The disclosed sensors' capabilities for long-term quantitative analysis and real-time on-body monitoring can also provide insightful information about Ca²⁺ and pH homeostasis in the human body. Owing to its miniaturization, system integration, and measurement simplification, the disclosed platform manifests a useful wearable sensing system that can be exploited for disease diagnosis where rapid analysis is desired for Ca²⁺ and pH in body fluids.

### Experimental Section With Respect To pH and Ca²⁺

Example Materials. Calcium ionophore II (ETH 129), bis(2-ethylehexyl) sebacate (DOS), sodium tetrakis[3,5-bis(trifluoromethyl)phenyl] borate (Na-TFPB), high-molecular-weight polyvinyl chloride (PVC), [tetrahydrofuran (THF), polyvinyl butyral resin BUTVAR B-98 (PVB), sodium chloride (NaCl), 3,4-ethylenedioxythiophene (EDOT), poly-(sodium 4-styrenesulfonate) (NaPSS), aniline, and moisture-resistant 100 μm-thick PET.

Example Fabrication of Electrode Array. The fabrication process may be the same or similar to that discussed above. The PET may be cleaned with isopropyl alcohol and O₂ plasma etching. An electrode array of 3.2 mm in diameter may be patterned via photolithography and may be thermally evaporated with 30/50 nm of Cr/Au, followed by lift-off in acetone. The electrode array may be additionally coated with 500 nm parylene C insulation layer (e.g., in a SCS Labcoter 2 Parylene Deposition System), and the 3 mm-diameter sensing electrode area may be defined via photolithography. The fabricated array may be further etched with O₂ plasma to remove the parylene layer at the defined sensing area. Then, 200 nm Ag may be deposited via thermal evaporation and lift-off in acetone. It is understood that other processes, dimensions, and materials may be used to fabricate the electrode array.

Preparation of Ca²⁺ Selective Sensors and pH Sensors. Ca²⁺-selective cocktail was prepared by dissolving 100 mg of 33:0.5:65.45:1 wt % ratio of PVC:NaTFPB:DOS:ETH129 in 660 μL THF. The surface of the Ca²⁺-selective electrodes was modified by galvanostatic electrochemical polymerization of 0.01 M EDOT with 0.1 M NaPSS at a constant current of 2 mA·cm-2 to produce polymerization charges of 10 mC. Ten μL (1.4 μL·cm-2) of Ca²⁺-selective cocktail was then drop-casted onto a PEDOT:PSS coated electrode and left to dry overnight in a dark environment. Aniline was distilled at a vapor temperature of 100° C. and a pressure of 13 mmHg before usage. PANI was polymerized in a 0.1 M aniline/0.1 M HCl solution. Au surface was first modified by depositing Au (50 mM HAuCl4 and 50 mM HCl) for 30 s at 0 V, followed by PANI deposition using cyclic voltammetry from −0.2 to 1 V for 25 cycles at 100 mV/s. It is understood that other processes, dimensions, and materials may be used to prepare the sensors.

Evaluation of Ca²⁺ and pH Sensors General Performances. General performance of Ca²⁺ sensors was tested under a 0.01 M acetate buffer solution (pH 4.6) containing varying Ca²⁺ concentrations unless stated otherwise. Interference study was performed by subsequent addition of chloride solutions containing various cations (2 mM H+, 2 mM NH4 +, 1 mM Mg²⁺, 8 mM K+, and 20 mM Na+) into a 1 mM Ca²⁺ solution. pH sensors were tested using McIlvaine's buffer with varying pH to characterize general performances of the sensors.

Interference study was conducted by subsequent addition of chloride solutions containing 1 mM Ca²⁺, 1 mM NH4 +, 1 mM Mg²⁺, 8 mM K+, and 20 mM Na+ into a Mcllvaine's buffer solution of pH 4.0. All measurements were paused while changing solutions, and measurements were done after 20 s waiting period.

Ex Situ Evaluation of Body Fluids. Urine, sweat, and tear samples were collected from volunteer subjects for off-body evaluation.

Sweat and urine were initially tested with ICP-MS to measure [Ca²⁺], and the results were compared with the sensor readings of same sweat and urine samples. Sweat samples were diluted four times with deionized water for ex situ evaluations using ICP-MS and wearable sensors. The results were converted back in Table 2 to reflect raw sweat Ca²⁺ concentrations. [Ca²⁺] measured by the sensor was computed using a calibration curve. The calibration curve was obtained from artificial body fluids containing 50 mM NaCl and 4 mM KCl with 0.25, 0.5, and 1 mM CaCl2 in 0.01 M acetate buffer. pH of the samples was measured with a commercial pH meter (Horiba LAQUA Twin pH meter B-713) and PANI-based pH sensors. PANI-based pH sensor measurement was obtained by using similar methods as the [Ca²⁺] measurement. pH values were computed from a calibration curve obtained from solutions containing 50 mM NaCl and 4 mM KCl with McMaine buffer of pH varying from 4 to 7. To further confirm sensor readings, raw sweat, urine, and tear samples were subsequently added with a fixed amount of Ca²⁺, and initial [Ca²⁺] was back-calculated based on the change in potential with concentration.

The relationship between potential change and logarithmic concentration was analyzed by comparing with a standard calibration curve obtained from FIG. 16c. As in measuring [Ca²⁺], HCl was subsequently added into the original body fluids, and final pH was verified by a commercial pH meter and compared with PANI-based sensor readings. In this case, a pH meter due to buffering capacity of body fluids measured the actual pH of the body fluids. The measured pH results were compared with a standard calibration line obtained from FIG. 14c.

In Situ Assessment of Sweat [Ca²⁺], pH, and Skin Temperature. On-body evaluation of sweat [Ca²⁺] and pH was performed in compliance with the protocol that was approved by the institutional review board at the University of California, Berkeley (CPHS 2014-08-6636). Five healthy male subjects, aged 20-30, were recruited. An electronically braked leg-cycle ergometer (Kettler E3 Upright Ergometer Exercise Bike) was used for stationary cycling trials. Subjects were told to bike for 30 minute at a constant workload cycle ergometry. Subject's forehead was wiped and cleaned with alcohol swab and gauze prior to wearing the sensor. Cycling protocol included a 5 minute ramp-up and a 20 minute biking at a power of 150 W, followed by a 5 minute cool-down session. Data are directly recorded in a mobile phone via a customized application. Sweat was simultaneously collected every 5 minute during cycling to compare on-body data with measurements from the ICP-MS and a pH meter. Collected sweat was diluted four times for ICP-MS measurements.

### Heavy Metals

The wearable sensing platform described herein can be configured with still additional or different features. For example, the wearable sensing platform may be adapted for heavy metal monitoring of body fluids. An aspect of the disclosure relates to a flexible and wearable microsensor array for simultaneous multiplexed monitoring of heavy metals in human body fluids, such as, by way of example, Zn, Cd, Pb, Cu, and Hg ions. The target analytes may be detected, by way of example, via electrochemical square wave anodic stripping voltammetry (SWASV) on Au and Bi microelectrodes.

In an example process, the oxidation peaks of these metals are calibrated and compensated by incorporating a skin temperature sensor. The wearable sensing platform sensor arrays may provide high selectivity, repeatability, and flexibility. Urine samples are collected for heavy metal analysis, and measured results from the microsensors are validated through inductively coupled plasma mass spectrometry (ICP-MS). Real-time on-body evaluation of heavy metal (e.g., zinc and copper) levels in sweat of human subjects by cycling is performed to examine the change in concentrations with time. The wearable sensing platform is configured to provide insightful information about an individual's health state such as heavy metal exposure and aid the related clinical investigations.

By way of background, human body fluids are composed of various electrolytes, proteins, metabolites, as well as heavy metals. A variety of heavy metals can be found in human body fluids (such as blood, sweat, and urine) and are closely related to human health conditions. For example, Cu and Zn are essential trace elements that can have detrimental effects on an individual's health when there is an excess or deficiency. High copper accumulation in human body can lead to Wilson's disease, heart and kidney failure, liver damage, brain disease and disorder, and even death in extreme cases, whereas low levels of copper can cause anemia and osteoporosis. A lethal form of diarrhea and pneumonia can occur when a body has low zinc concentrations, whereas high levels of zinc can be toxic enough to cause liver damage, and even decrease cardiac functionality and pancreatic enzyme count in cases of prolonged exposure.

Additionally, cadmium, lead, and mercury exhibit toxic effects on human body systems including the nervous, immunological, and cardiovascular systems. High levels of cadmium exposure can lead to fatal respiratory tract, liver, and kidney problems. On the other hand, lead poisoning can slow down growth and cause other developmental delay as well as irritability, increased violent behavior, learning difficulties, fatigue, loss of appetite, and hearing loss for children and cause memory loss, infertility, high blood pressure, and decline in mental functioning for adults. Further, mercury poisoning leads to many diseases such as Hunter-Russell syndrome, Minamata disease, and acrodynia, to name a few. Therefore, determining one's exposure to such heavy metals can offer important insights into a person's health. Human sweat and urine are known to be the most important sources for detoxification of heavy metals; therefore, examination of sweat and urine heavy metals can assist toxicological and therapeutic studies.

Conventionally, detection of heavy metals in body fluids is challenging due to their extremely low concentrations (on the order of μg/L). Conventional heavy metals analysis procedures, including atomic absorption spectroscopy (AAS) or inductively coupled plasma mass spectrometry (ICP-MS), rely on inconvenient, bulky and expensive analytical instruments. Further, such instruments may be unavailable in many regions of the world.

The effective preconcentration/deposition step and advanced electrochemical measurements of the accumulated analytes make anodic stripping analysis a highly sensitive and effective electroanalytical technique. The stripping-voltammetric measurements of trace metals in sweat have been reported using collected sweat samples. Disadvantageously, such use of collected sweat may be subject to inaccuracy due to sample contamination and sweat evaporation. In addition, such techniques do not yield real-time information on dynamic events. Given the importance or toxicity of a variety of heavy metals to the individual's health states, it is very attractive to perform simultaneous multiplexed screening of heavy metals in sweat and do proper signal calibrations to ensure accurate measurements.

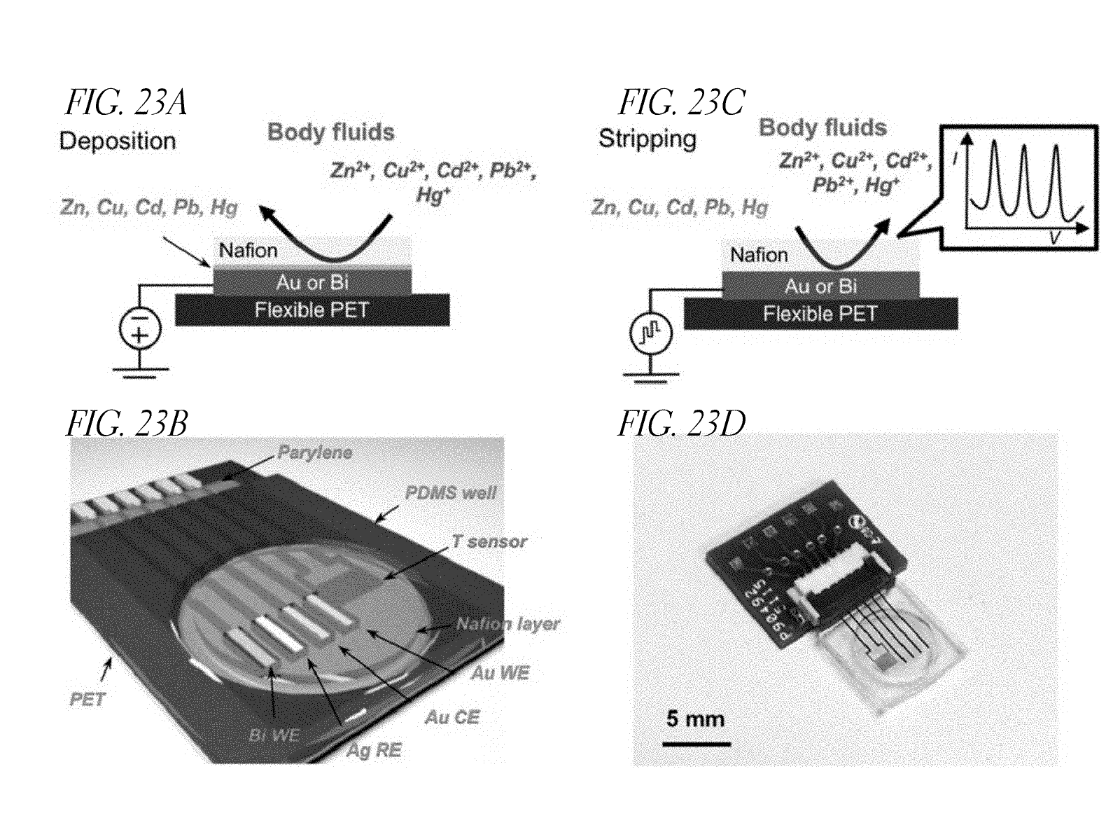

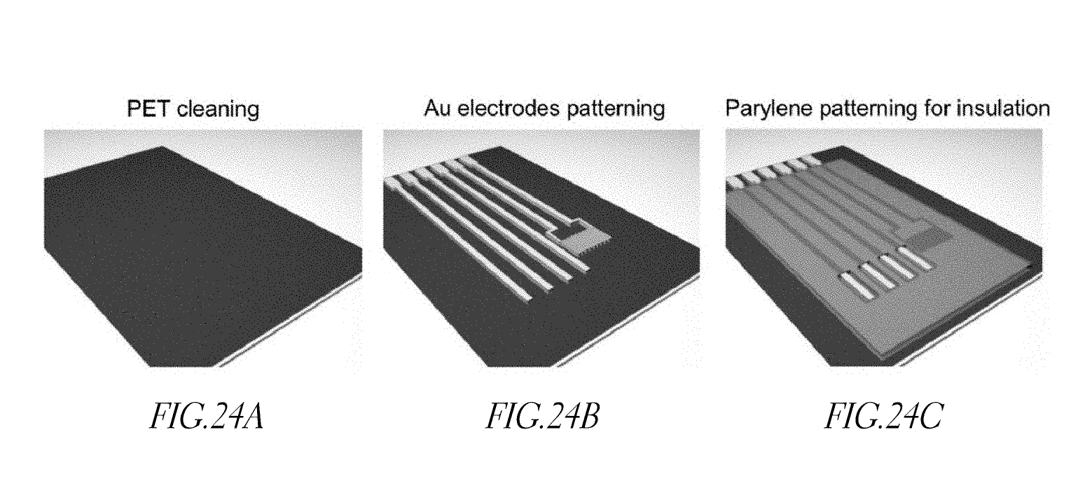

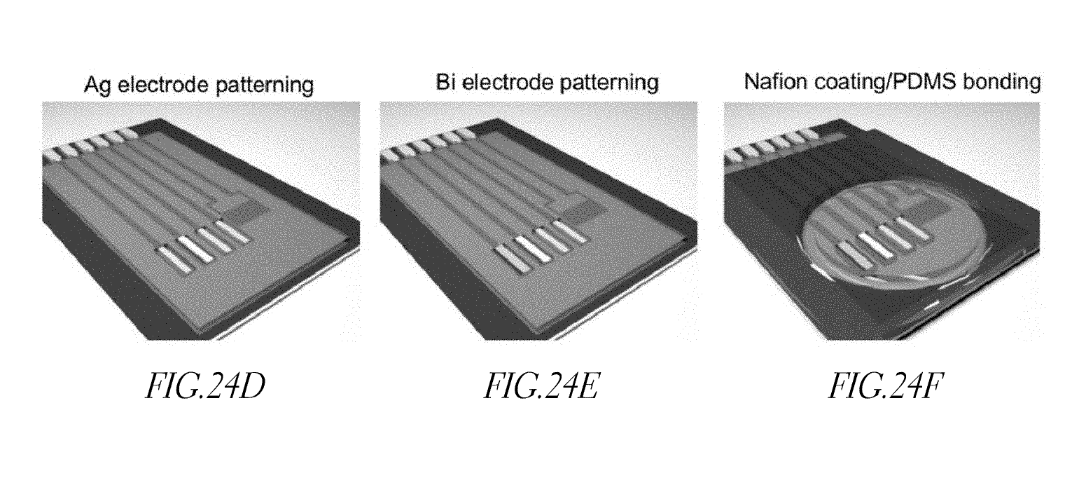

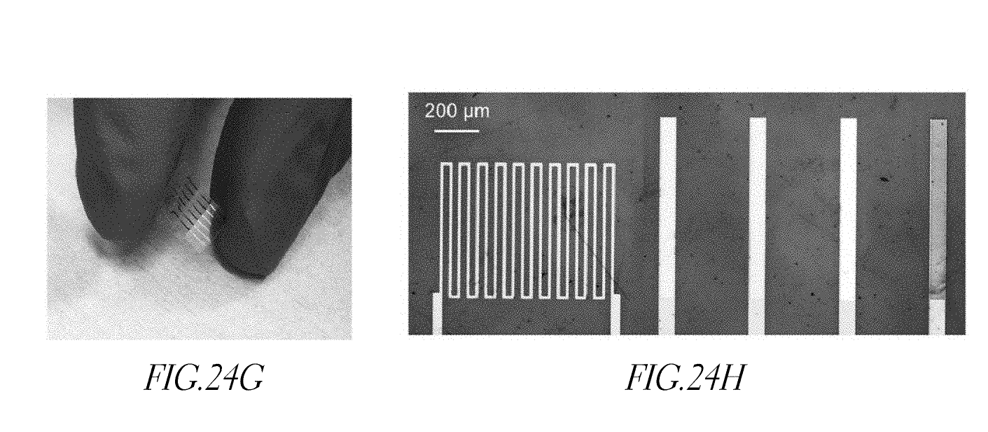

Disclosed herein is a flexible multiplexed trace metals monitoring device to extract useful information on heavy metal levels in body fluids such as sweat and urine. The device can also be used as a wearable device for real-time monitoring of the heavy metals in human sweat. A microsensor array is utilized to simultaneously and selectively measure multiple heavy metals (e.g., Zn, Cd, Pb, Cu, and/or Hg) using, by way of example, square wave anodic stripping voltammetry (SWASV), as well as skin temperature to calibrate heavy metal sensors' readings in real-time (FIG. 23a). The microsensor array includes multiple sensors (FIGS. 24c-24d). In the example illustrated in FIG. 23b, the array includes four micro-electrodes: biocompatible gold and bismuth working electrodes (WE), a silver reference electrode (RE), and a gold counter/auxiliary electrode (CE). In addition, a resistance-based skin temperature sensor based on evaporated Cr/Au microlines is integrated into the system (as similarly discussed elsewhere herein) to compensate the sensors' readings since temperature has a significant influence on the electrochemical processes.

Furthermore, on-body measurements of sweat trace metals during exercise are performed by implementing the integrated sensors directly on human skin. Such real-time assessment of heavy metals in sweat can give early warnings of heavy metal exposure. As similarly discussed elsewhere herein (see, e.g., FIG. 7), the assessments may be communicated to a user device (e.g., a mobile phone) for presentation to the user, and may be shared with others' such as the user's doctor(s) who may use the information to track the user's progress and to identify any urgent issues.

The working electrode is selected to provide for successful stripping analysis. The ideal material for working electrode should offer an effective preconcentration, a favorable redox reaction of the target metal, reproducible and renewable surface, and a low background current over a wide potential range. Although mercury has been the most explored electrode for many stripping applications, it is not desired for wearable biosensors given its toxicity and volatility. However, bismuth and gold electrodes, by way of example, provide good stripping voltammetric performance and biocompatibility and so are employed in developing the disclosed wearable biosensors for heavy metals analysis.

The microsensors arrays are optionally fabricated on a flexible polyethylene terephthalate (PET) substrate through a procedure involving multiple steps of photolithography, evaporation (Cr/Au, Ag, Bi), and lift-off as illustrated in FIG. 24. In this example, a 500 nm layer of parylene C is chosen as an insulation layer to ensure reliable measurement by preventing electrical contact of the conducting metal lines with body fluids and skin. Photolithography and O₂ plasma etching are used to define the electrode area (e.g., 100 μm×1200 μm). After patterning the Ag reference, Bi and Au working electrodes, a thin Nafion coating is used as an antifouling layer to minimize or reduce biofouling of surface-active macromolecules such as proteins. A polydimethylsiloxane (PDMS) well (e.g., 6 mm diameter and 1 mm thickness) was also bonded on top of the sensor array by soft lithography and O₂ plasma etching. This ensures sweat accumulation (e.g., 20-30 μL) such that a stable and reliable stripping analysis can be performed. The microelectrodes array is then connected to a potentiostat through an interface consisting of a flexible printed circuit (FPC) connector (FIG. 23c).

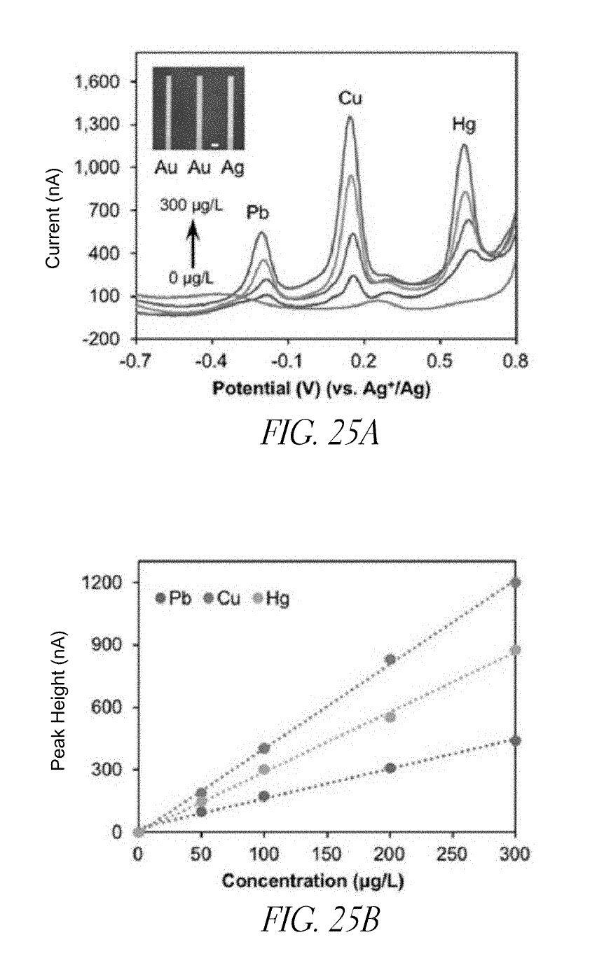

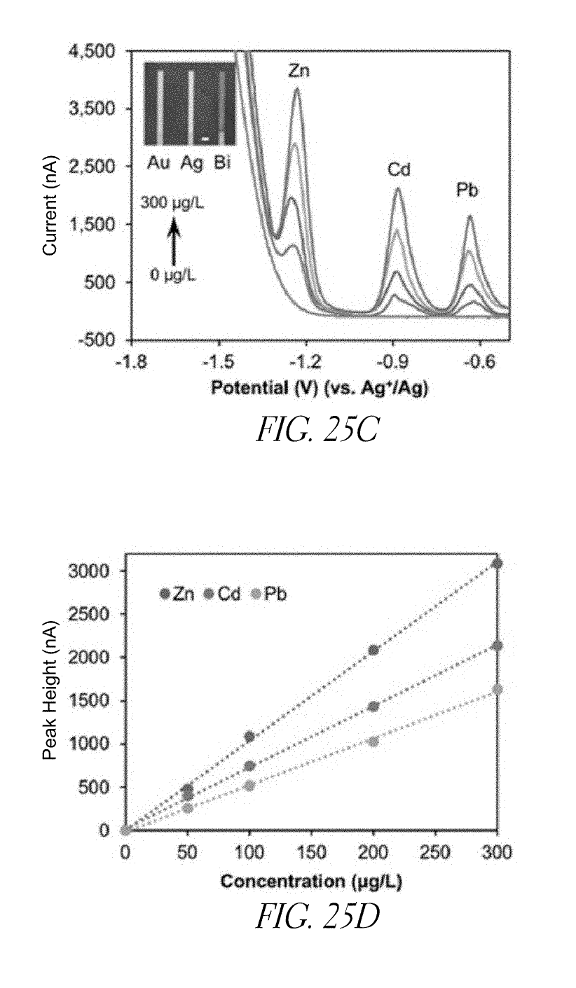

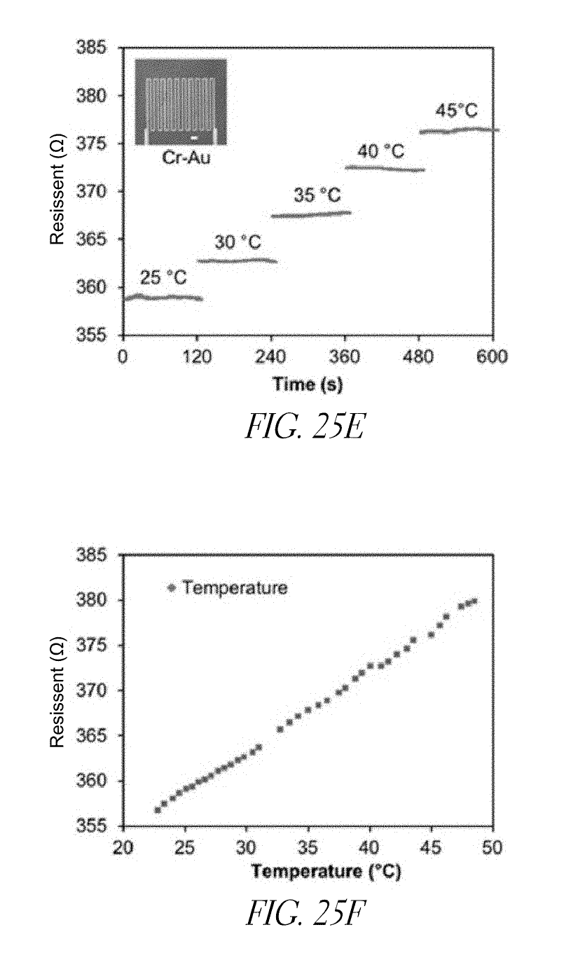

As aforementioned, Au microelectrodes offer excellent biocompatibility and a wide operational potential window owing to their high stability. Au is an excellent electrode material for Pb, Cu, and Hg stripping, although other materials may be used. In order to investigate the relationship between the concentration of heavy metals and the response of SWASV, the voltammograms are recorded using a 0.01 M acetate buffer solution (pH 4.6) containing 50 mM NaCl (to mimic human sweat) with an addition of 50-100 μg/L heavy metals after every trial. As illustrated in FIG. 25a, three distinct peaks near −0.2, 0.2, and 0.6 V which correspond to oxidation of Pb, Cu, and Hg, respectively, are observed for a Au microelectrode.

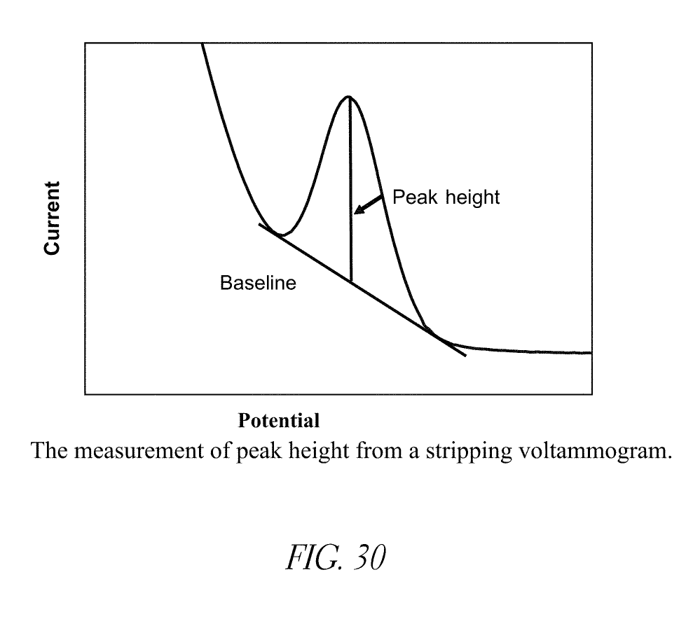

A linear relationship between the peak height (current amplitude measured from the baseline as illustrated in FIG. 30) and analyte concentration with sensitivities of 1.4, 4.1, and 2.9 nA·L/μg for Pb, Cu, and Hg, respectively, is demonstrated in FIG. 25b. No distinct oxidation peak of Cd was observed in experiments using Au microelectrodes. In addition, although it is also possible to obtain the oxidation peak for Zn using Au microelectrodes, Zn stripping results in strong hydrogen evolution because Zn deposits and strips at a very negative potential (close to the hydrogen evolution potential) compared to other trace metals. On the other hand, Bi electrodes offer excellent biocompatibility with high sensitivity, and low sensitivity to dissolved oxygen. Despite these advantages, Bi electrodes are not ideal for stripping Cu and Hg due to its relatively lower oxidation potential (˜−0.3 V). Hence, in this example, a Bi microelectrode is only utilized for Cd, Pb, and Zn detection.

FIG. 25c shows three distinct oxidation peaks for Zn, Cd, and Pb near −1.2, −0.9, and −0.6 V, respectively, from a Bi microelectrode. The corresponding sensitivities for Zn, Cd, and Pb are 10.4, 7.1, and 5.4 nA·L/μg, respectively (FIG. 25d). Note that high current at a very negative potential before the zinc peak is observed due to the hydrogen evolution. FIGS. 25e and 25f illustrate the linear response of a temperature sensor in physiological skin temperature range with a sensitivity of ˜0.24%/° C. (normalized to the resistance at 20° C.).

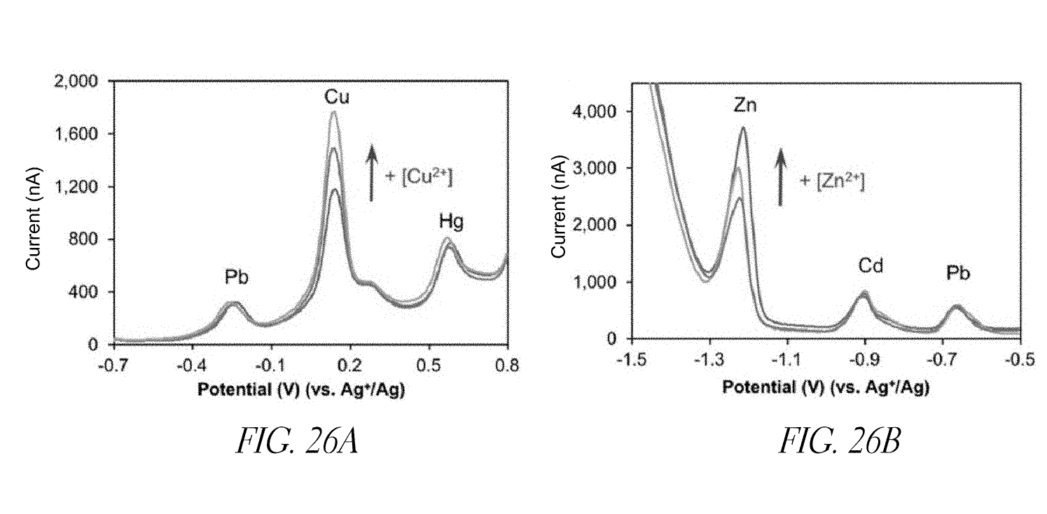

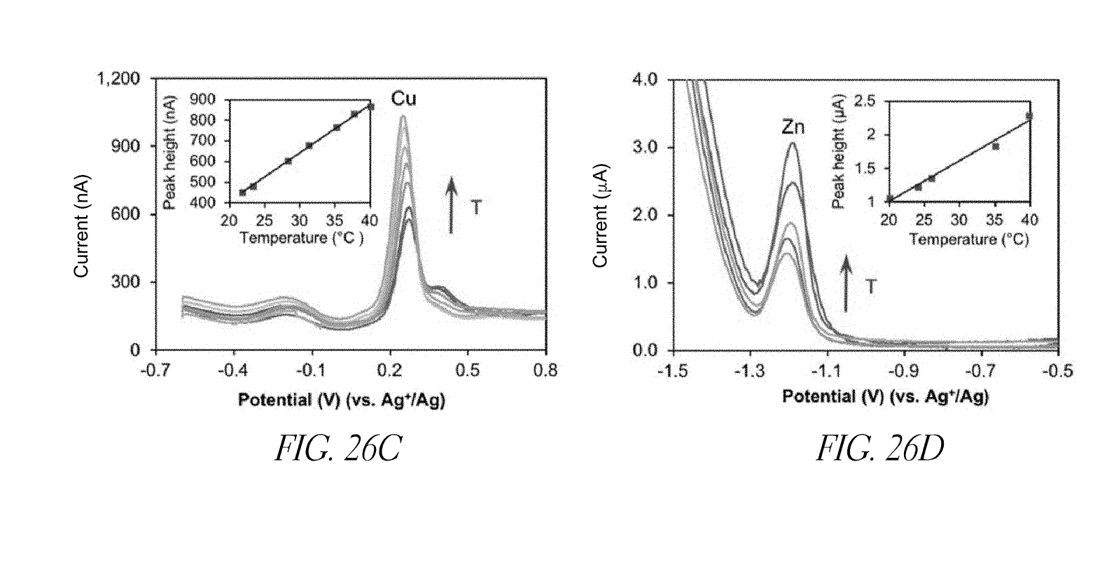

The selectivity of the Bi and Au based microsensors are advantageous for the analysis in biofluids. Given the relatively high concentration of sweat Cu and Zn (on the order of hundreds μg/L), an interference study on Au and Bi based microsensors is implemented by varying Cu and Zn concentrations, respectively. As illustrated in FIG. 26a, while the Cu concentration increases from 200 to 250 and 300 μg/L, the Cu current peak significantly increases while the current peaks of Pb and Hg (with a significantly lower concentration of 100 μg/L) remain unchanged. Similarly, while the Zn concentration is raised from 200 to 250 and 300 μg/L, the oxidation peak of Zn increases linearly with its concentration while there are no clear interferences on the peaks of Cd and Pb (with a concentration of 100 μg/L) (FIG. 26b, which illustrates the graph for Cu, which is above the graph for Hg, which is above the graph for Pb). Likewise, when varying concentrations of Cd, Pb, and Hg, no obvious interferences for the measurements of the other metals were observed on the Bi and Au microelectrodes. Such high selectivity of stripping analysis among the target heavy metals is advantageous for the on-body test given the high complexity of human body fluids.

Another significant aspect is the influence of temperature on the responses of biosensors. Skin temperature and environmental temperature can have direct influence on metabolite sensors; hence, temperature compensation advantageously ensures accurate readings of the sensors. To this end, SWASV responses of the microsensors are investigated by gradually increasing the temperature of the solution containing Cu(II) and Zn(II) from 20 to 40° C. As shown in FIG. 26c (which illustrates the graph for Zn, which is above the graph for Cd, which is above the graph for Pb) and 26d, in this example responses of both Au and Bi electrodes increase almost linearly with increasing solution temperature over the range with a slope of 5.5%/° C. and 6%/° C., respectively, relative to the peak height at 20° C. The increase in the sensors' responses reflects the elevated mass transport and electrochemical deposition and stripping kinetics.

The integration of a temperature sensor into the microsensor array enables real-time temperature compensation to ensure an accurate and a reliable heavy metal detection. The repeatability of Au and Bi based microsensors for stripping analysis of Cu and Zn was examined by recording the stripping voltammograms under the same condition mentioned above in a solution containing 150 μg/L Cu and Zn, respectively.

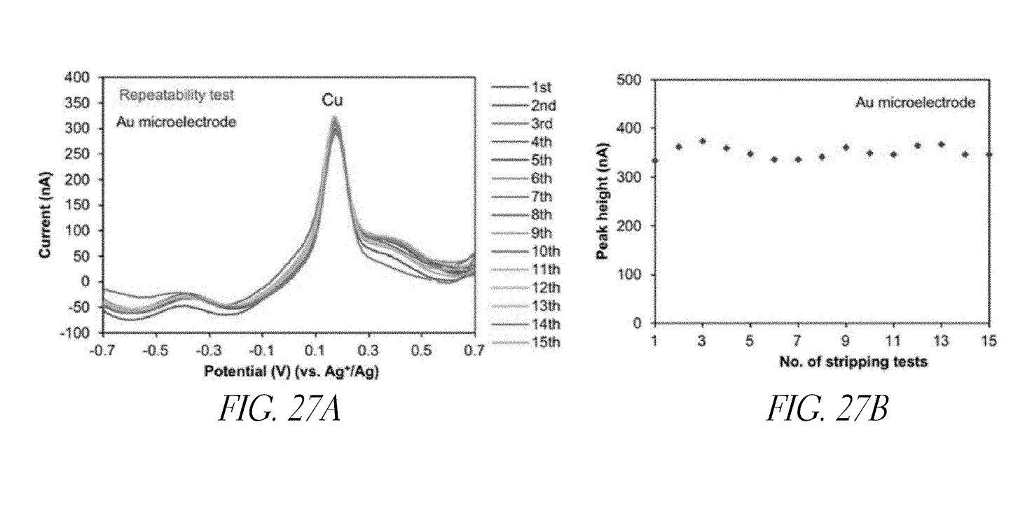

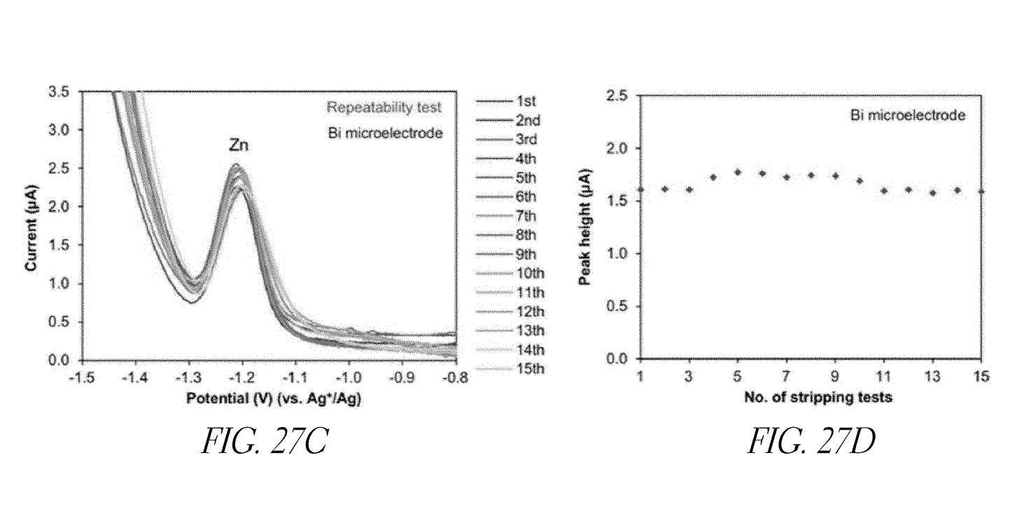

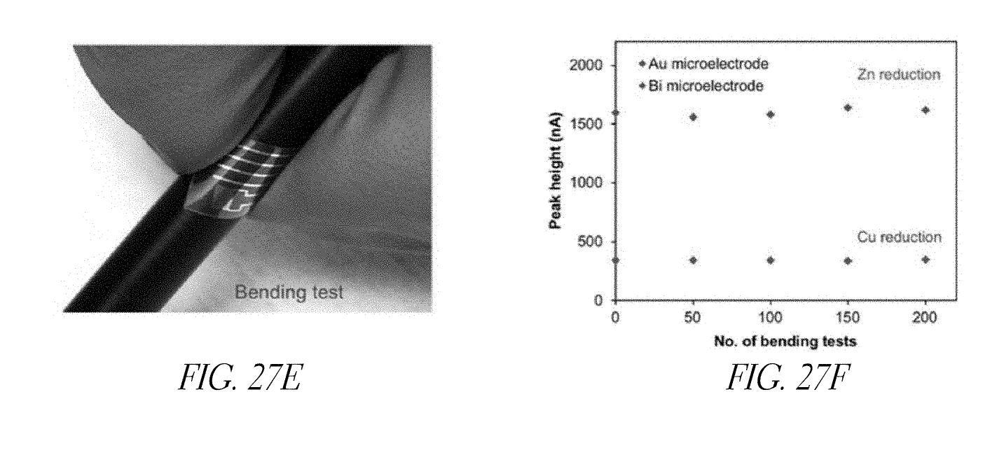

FIGS. 27a and 27b illustrate an example of 15 continuous stripping voltammograms and corresponding peak height for Cu detection using the same Au microelectrode. The relative standard deviation (RSD) of the peak measurements in this example is 3.6%. Similarly, FIGS. 27c and 27d illustrate the stripping voltammograms and corresponding peak height for Zn detection using a Bi microelectrode with a 4.4% RSD for the peak height measurements. It should be noted that the Au-based sensors remain stable even after 100 times measurements while the performance of Bi-based sensors gradually decreases after 15 times test due to the consumption of Bi film.

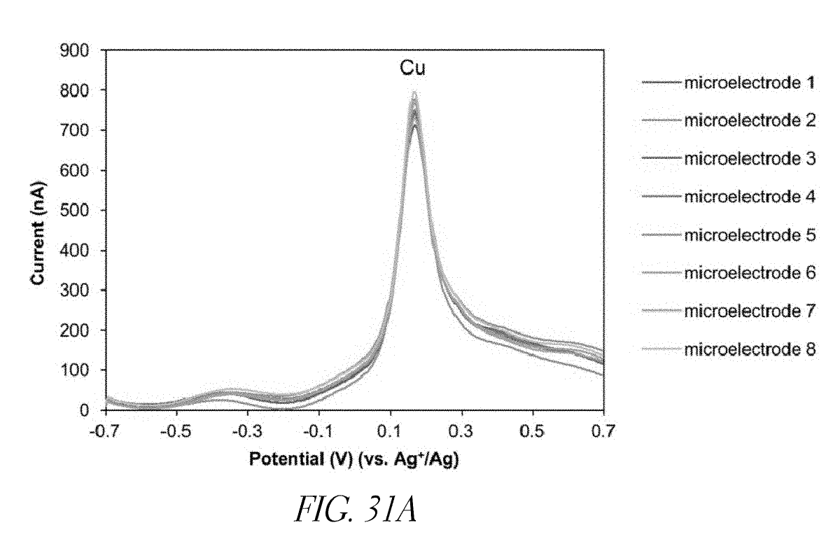

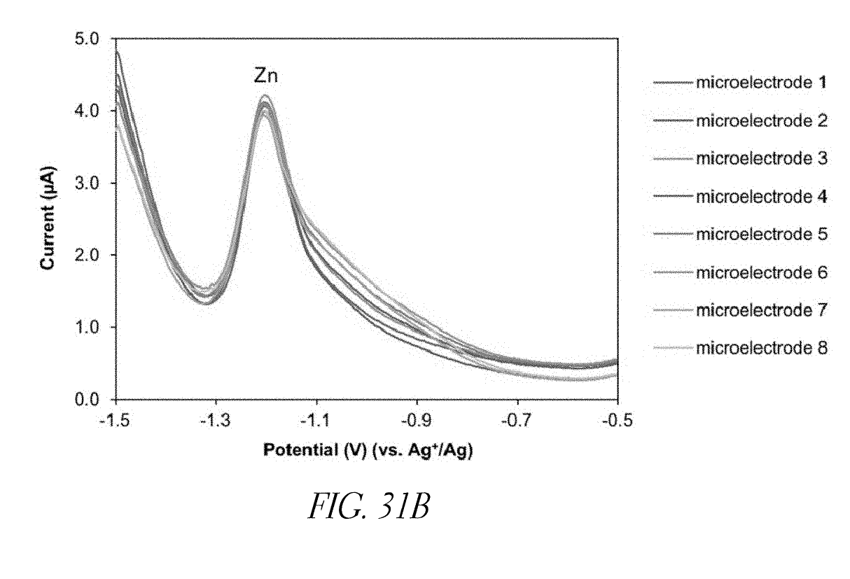

In addition, the reproducibility of different microsensor arrays for heavy metal analysis was examined. As demonstrated in FIGS. 31a-31b, the corresponding RSDs for Cu and Zn analysis using eight different microsensor arrays are 4.6% and 3.9%, respectively. Unlike the most common type of electroplated thick Bi film electrode, the Bi microelectrode displays excellent repeatability due to the high uniformity of the evaporated Bi film. The use of micro/nanoelectrodes can greatly enhance the mass transport rate due to small size. Additionally, the magnetic stirring commonly used for SWASV of regular macroscale electrodes to obtain repeatable and reliable data is avoided in our study. This represents a significant advantage for the use of microelectrodes on wearable sensing devices.

The wearable microsensor array preferably are able to withstand mechanical deformation during vigorous physical exercise. The flexibility was investigated by monitoring the peak heights of stripping performance of the sensor array after mechanical bending (radii of curvature is 3.2 mm) (FIG. 27e). As demonstrated in FIG. 27f, no obvious variations (relative standard deviations are 1% and 1.9% for Cu and Zn detection, respectively) are observed for the stripping data even after 200 times bending tests in one example. This indicates that the flexible microsensor array is robust enough for on-body test to endure the excess deformation during physical exercise.

A stable and a reliable performance of biosensors in biofluids is desirable for practical usage. To demonstrate such capability of the microsensor array (not only for on-body real-time monitoring but also for off-body analysis of different biofluids), sweat and urine samples are collected from volunteer subjects for off-body measurements. The physiological levels of heavy metals in human sweat and urine are relatively low (<1 mg/L). Specifically, human sweat contains 100-1000 μg/L of Zn and Cu while the concentrations of Pb, Cd, and Hg usually fall below 100 μg/L. Because of the relatively high concentrations of free Cu and Zn ions, off-body measurements showed visible oxidation peaks in sweat and urine samples of all the subjects. For Cu stripping, 15 seconds deposition time was used on the Au microelectrodes to minimize peak distortion and electrode fouling. In addition, the permselective/protective Nafion coating is found to be beneficial in addressing the challenge of biofouling due to the surface-active compounds in complex human biofluids. It helps to enhance oxidation peaks of targeted trace metals and allows direct detection in human sweat and urine samples.

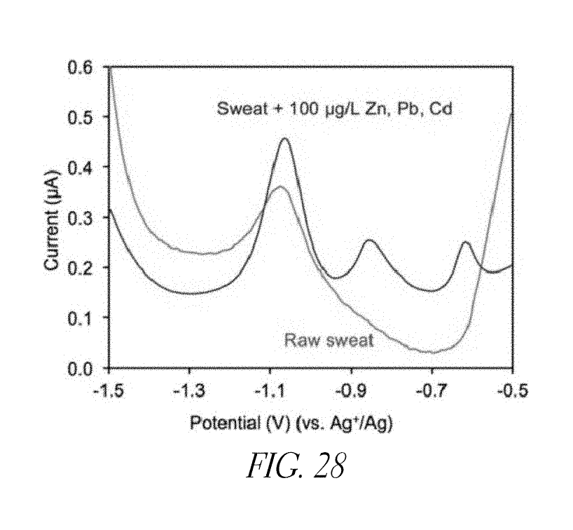

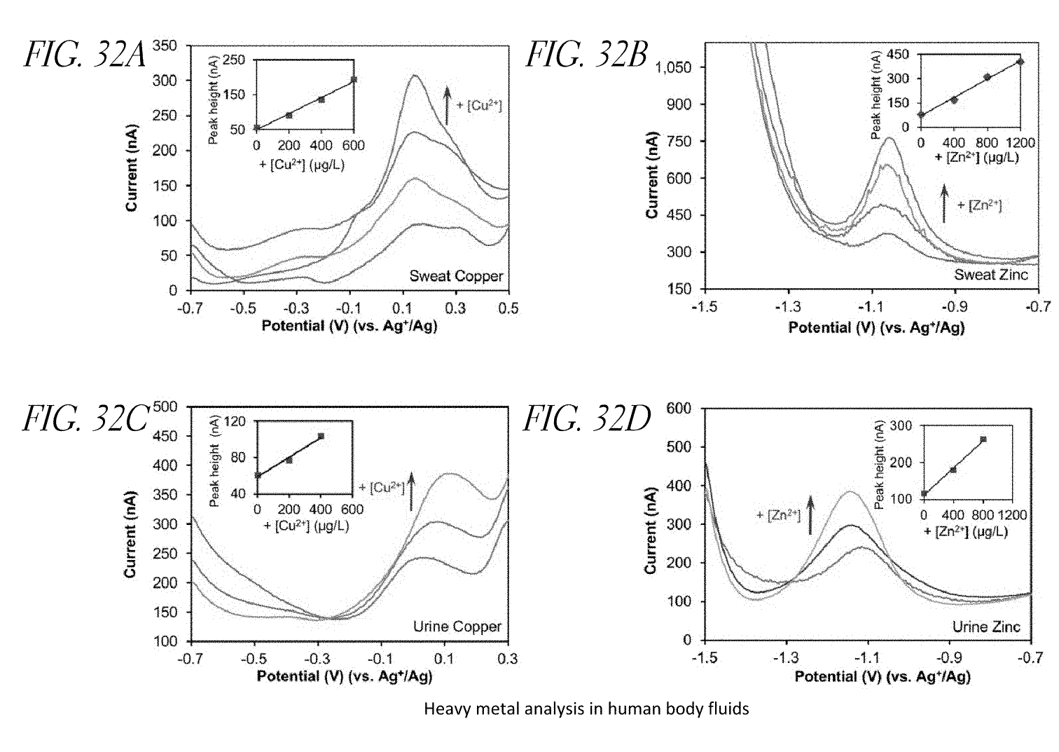

Cu and Zn levels in sweat (FIGS. 32a and 32b) and urine samples (FIGS. 32c and 32d) were determined by manual addition of target metal and back-calculating sweat concentrations as detailed in Experimental Section. Such a calibration method could minimize the potential influence of Cu-Zn intermetallic compounds formed on Bi electrodes (due to the high Cu levels in biofluids). Sweat samples were collected from subjects during a constant load indoor cycling exercise, while human urine samples were obtained from volunteer subjects. The measured concentrations of Cu and Zn in sweat and urine are consistent with earlier findings reported in the literature. Concentrations of Cu and Zn in biofluids were also validated with inductively coupled plasma mass spectrometry (ICP-MS). Table 3 illustrates the comparison between the measured concentrations of Cu and Zn in sweat and urine samples by using the microsensor array and by the ICP-MS method. No significant difference was observed between these two methods. This confirms that the microsensor array can be used for accurate measurement of heavy metals in sweat and urine. Although no obvious oxidation peaks of Pb, Cd, or Hg are observed due to their low concentration in sweat and urine of normal subjects, it is possible to detect them using the microelectrodes in those who are subjected to heavy exposure. It has been reported that Pb and Cd can reach 200-300 μg/L in sweat for the subjects undergoing heavy exposure. FIG. 28 depicts the sensitivity of microsensors when heavy metals reach toxic levels in raw sweat (with the addition of 200 μg/L target analytes including Zn, Pb, and Cd).

TABLE 3
Measurement Results for Cu and Zn Content in Human
Sweat and Urine Samples

| | Measurement results from | Measurement results from |
|---|---|---|
| Sample | the microsensor arrays (μg/L) | ICP-MS (μg/L) |
| sweat Cu | 249 | 267 |
| sweat Zn | 274 | 290 |
| urine Cu | 571 | 601 |
| urine Zn | 624 | 634 |

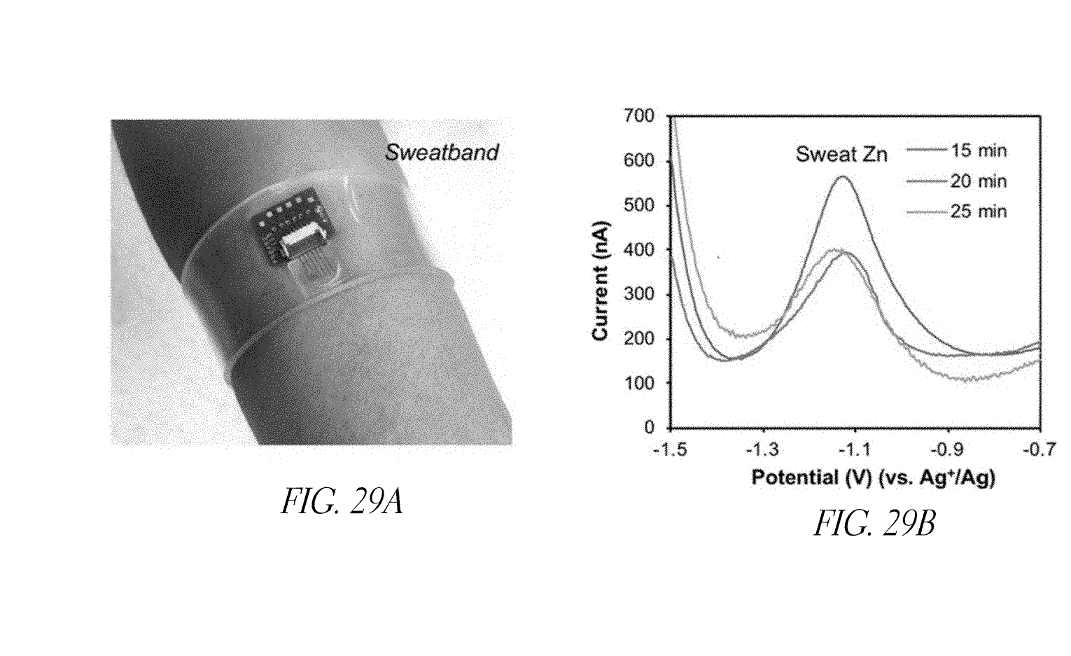

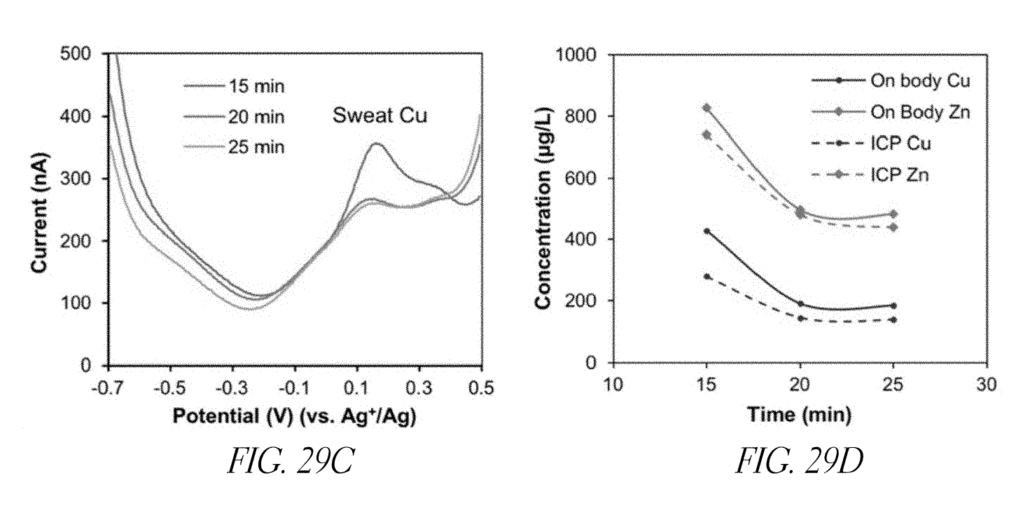

On-body heavy metals monitoring was performed during a constant-load exercise on a cycle ergometer. The protocol involved a 5 minute ramp-up, 30 minute cycling at 150 W, and a 5 minute cool-down. The microsensor arrays were packaged in a wristband (FIG. 29a) which could be comfortably worn by the subject. During the exercise, the skin temperature, sweat Cu, and sweat Zn were monitored by using an electrochemical potentiostat. FIGS. 29b and 29c illustrate the calibrated stripping voltammograms for heavy metal detection recorded by the microsensor array at different time during the exercise of a volunteer subject. The current readings were calibrated according to the real-time temperature information (stabilized at ˜34° C. during the measurement period). It can be seen that the initial concentrations of sweat copper and sweat zinc are relatively high. As exercise continues, Cu and Zn concentrations decrease and gradually reach stable levels during the rest of the exercise. This general trend is also consistent with the ICP-MS data obtained from collected sweat samples in every 5 minute period (FIG. 29d). The slightly higher value from the on-body measurement is likely due to enhanced diffusion in the sweat sample during the deposition period caused by the subject's vigorous motion.

Thus, a wearable and flexible microsensor array is disclosed that can perform simultaneous and selective detection of multiple heavy metals (e.g., Zn, Cd, Pb, Cu, and Hg) noninvasively. The flexible microsensor arrays display very good repeatability and stability for heavy metal analysis. A temperature sensor is utilized for real-time compensation of the signals to ensure accurate and reliable measurements. The microsensor array has been successfully used to accurately and selectively monitor heavy metal levels in human body fluids such as sweat and urine. The disclosed microsensor array device greatly expands the panel of analytes for noninvasive wearable biosensing. For example, the microsensor array device may be used to monitor heavy metal exposure and aid in related clinical investigations.

### Experimental Section With Respect To Heavy Metals

Example Materials. Moisture-resistant polyethylene terephthalate (PET), 100 μm thick, zinc, cadmium, lead, copper, and mercury standard AAS solutions (1000 mg/L in nitric acid), acetate buffer, sodium chloride (NaCl), and Nafion 117 solution (5 wt %).

Fabrication of Electrode Arrays. An example fabrication process of the electrode arrays is illustrated in FIG. 24. The sensor arrays on PET were patterned by photolithography using positive photoresist (Shipley Microposit S1818) followed by 30/100 nm Cr/Au deposited via electron beam (e-beam) evaporation and lift-off in acetone. A 500 nm parylene C insulation layer was then deposited in a SCS Labcoter 2 Parylene Deposition System. Subsequently, photolithography was used to define the electrode area (100 μm×1200 μm) followed by O₂ plasma etching for 450 s at 300 W to completely remove parylene at the defined electrode areas. E-beam evaporation was then performed to pattern 180 nm Ag on the electrode areas followed by lift-off in acetone. Photolithography and e-beam evaporation were used to pattern 300 nm Bi on the electrode areas followed by lift-off in acetone. One microliter of Nafion 117 solution was then drop casted onto the microsensor array and dried for 2 hours. A polydimethylsiloxane (PDMS) well (6 mm diameter and 1 mm thickness) was fabricated using a soft lithography process. It was bonded to the flexible PET substrate using O₂ plasma etching treatment on the PDMS surface for 90 s at 90 W. The PDMS well allows the accumulation of sufficient sweat volume for stripping analysis. The microelectrodes array was connected to the potentiostat through an interface consisting of a flexible printed circuit connector.

Characterizations of the Microsensor Arrays. Square wave anodic stripping voltammetry (SWASV) was employed to characterize the electrochemical stripping of heavy metals on the microsensor arrays. In order to evaluate the performance of Bi electrodes, a deposition potential of −1.5 V (vs Ag+/Ag) was applied for 180 s, followed by a SWASV scan to a final potential of −0.5 V (vs Ag+/Ag) at a frequency of 60 Hz, an amplitude of 40 mV, and a potential step of 4 mV in 0.01 M acetate buffer (pH 4.6) containing 50 mM NaCl. In order to evaluate the performance of Au electrodes, a deposition potential of −0.7 V (vs Ag+/Ag) was applied for 120 s (15 s for repeatability tests and detection in biofluids), followed by a SWASV scan to a final potential of 0.8 V (vs Ag+/Ag) at a frequency of 60 Hz, an amplitude of 40 mV, and a potential step of 4 mV in 0.01 M acetate buffer (pH 4.6) containing 50 mM NaCl. Mechanical deformation was tested by repeatedly bending (radii of curvature is 3.2 mm) the microelectrodes array for 200 times.

Off-Body Calibration and Validation in Human Sweat and Urine. Sweat samples were collected directly from the forehead and the arm of volunteer subjects during their constant load (150 W) cycling exercise. The subjects' skin was cleaned with alcohol swabs and gauze before the exercise and after every sweat collection. Urine samples were collected from the same volunteer subjects. And 50 μL human sweat and urine samples were used for the off-body measurement. The level of heavy metals was estimated by SWASV through standard addition (1-2 μL each time) of 100 mg/L Zn or Cu standard solutions. It should be noted that, in some cases, the peak positions slightly shifted after the standard addition due to the greatly changed heavy metal concentrations. The measurement results of biofluid heavy metal levels from the microsensors were compared with the data measured directly through an ICP Optima 7000 DV instrument.

Real Time On-Body Heavy Metal Analysis. On-body evaluation of the microsensor arrays was performed in compliance with the protocol that was approved by the institutional review board at the University of California, Berkeley (CPHS 2014-08-6636). Five healthy subjects (all males), aged 20-40, were used. An electronically braked leg-cycle ergometer (Kettler E3 Upright Ergometer Exercise Bike) was used for cycling trials. The subjects' skin was cleaned with alcohol swabs and gauze before sensors were worn on-body. A constant workload cycling regimen was used in which subjects were cycling at 50 W with 50 W increments every 150 s up to 150 W, and then cycling at 150 W for 30 min. The 5 minute cool down section involved cycling with decreased power output by 50 W every 150 s. During the exercise, on-body analysis was recorded using a Gamry electrochemical potentiostat (PCI4/G300). The on-body measurement results were calibrated using the measured skin/environment temperature at the same time. Such calibration eliminated the errors of stripping signals resulted from temperature variations. The heavy metal concentrations from on-body tests were roughly estimated using a coefficient factor obtained from an off-body standard addition method shown in FIG. 32 (based on the sweat sample collected from previous running test of the same subject). Three sweat samples were also collected in a 5 minute period (10-15 min, 15-20 min, 20-25 min) followed by skin cleaning during the same exercise and tested with ICP-MS.

### REFERENCES CITED

Kim, D. H. et al. Epidermal electronics. Science 333, 838-843 (2011).

Lipomi, D. J. et al. Skin-like pressure and strain sensors based on transparent elastic films of carbon nanotubes. Nature Nanotech. 6, 788-792 (2011).

Takei, K. et al. Nanowire active-matrix circuitry for low-voltage macroscale artificial skin. Nature Mater. 9, 821-826 (2010).

Wu, W. et al. Piezoelectricity of single-atomic-layer MoS2 for energy conversion and piezotronics. Nature 514, 470-474 (2014).

McAlpine, M. C., Ahmad, H., Wang, D., Heath, J. R. Highly ordered nanowire arrays on plastic substrates for ultrasensitive flexible chemical sensors. Nature Mater. 6, 379-384 (2007).

Kaltenbrunner, M. et al. An ultra-lightweight design for imperceptible plastic electronics. Nature 499, 458-463 (2013).

Xu, S. et al. Soft microfluidic assemblies of sensors, circuits, and radios for the skin. Science 344, 70-74 (2014).

Wang, C. et al. User-interactive electronic skin for instantaneous pressure visualization. Nature Mater. 12, 899-904 (2013).

Liang, J., Li, L., Niu, X., Yu, Z., Pei, Q. Elastomeric polymer light-emitting devices and displays. Nature Photon. 7, 817-824 (2013).

Kim, D. H., Ghaffari, R., Rogers, J. A. Flexible and stretchable electronics for biointegrated devices. Annu. Rev. Biomed. Eng. 14, 113-128 (2012).

Patel, S., Park, H., Bonato, P., Chan, L., Rodgers, M. A review of wearable sensors and systems with application in rehabilitation. J. Neuroeng. Rehab. 9, 21 (2012).

Bandodkar, A. J., Wang, J. Non-invasive wearable electrochemical sensors: a review. Trends Biotechnol. 32, 363-371 (2014).

Sonner, Z. et al. The microfluidics of the eccrine sweat gland, including biomarker partitioning, transport, and biosensing implications. Biomicrofluidics 9, 031301 (2015).

Schazmann, B. et al. A wearable electrochemical sensor for the real-time measurement of sweat sodium concentration. Anal. Methods 2, 342-348 (2010).

Jia, W. et al. Electrochemical tattoo biosensors for real-time noninvasive lactate monitoring in human perspiration. Anal. Chem. 85, 6553-6560 (2013).

Coyle, S. et al. BIOTEX—biosensing textiles for personalised healthcare management. IEEE Trans. Inf. Technol. Biomed. 14, 364-370 (2010).

Bandodkar, A. J. et al. Epidermal tattoo potentiometric sodium sensors with wireless signal transduction for continuous non-invasive sweat monitoring. Biosens. Bioelectron. 54, 603-609 (2014).

Rose, D. P. et al. Adhesive RFID sensor patch for monitoring of sweat electrolytes. IEEE Transact. Biomed. Eng. 62, 1457-1465 (2015).

Boysen, T. C., Yanagawa, S., Sato, F., Sato, K. A modified anaerobic method of sweat collection. J. Appl. Physiol. 56, 1302-1307 (1984).

Speedy, D. B., Noakes, T. D., Schneider, C. Exercise-associated hyponatremia: a review. Emerg. Med. 13, 17-27 (2001).

Talary, M. S., Dewarrat, F., Huber, D., Caduff, A. In vivo life sign application of dielectric spectroscopy and non-invasive glucose monitoring. J. Non-Cryst. Solids 353, 4515-4517 (2007).

Derbyshire, P. J., Barr, H., Davis, F., Higson, S. P. Lactate in human sweat: a critical review of research to the present day. J. Physiol. Sci. 62, 429-440 (2012).

Webb, R. C. et al. Ultrathin conformal devices for precise and continuous thermal characterization of human skin. Nature Mater. 12, 938-944 (2013).

Sprigle, S., Linden, M., McKenna, D., Davis, K., Riordan, B. Clinical skin temperature measurement to predict incipient pressure ulcers. Adv. Skin Wound Care 14, 133-137 (2001).

Zhu, J., Qin, Y., Zhang, Y. Preparation of all solid-state potentiometric ion sensors with polymer-CNT composites. Electrochem. Commun. 11, 1684-1687 (2009).

Jobst, G. et al. Thin-film microbiosensors for glucose-lactate monitoring. Anal. Chem. 68, 3173-3179 (1996).

Nadel, E. R., Bullard, R. W., Stolwijk, J. A. Importance of skin temperature in the regulation of sweating. J. Appl. Physiol. 31, 80-87 (1971).

Cage, G. W., Wolfe, S. M., Thompson, R. H., Gordon, R. S. Jr. Effects of water intake on composition of thermal sweat in normal human volunteers. J. Appl. Physiol. 29, 687-690 (1970).

Patterson, M. J., Galloway, S. D. R., Nimmo, M. A. Variations in regional sweat composition in normal human males. Exp. Physiol. 85, 869-875 (2000).

Messonnier, L. A. et al. Lactate kinetics at the lactate threshold in trained and untrained men. J. Appl. Physiol. 114, 1593-1602 (2013).

Buono, M. J., Lee, N. V., Miller, P. W. The relationship between exercise intensity and the sweat lactate excretion rate. J. Physiol. Sci. 60, 103-107 (2010).

Barr, S. I. Effects of dehydration on exercise performance. Can. J. Appl. Physiol. 24, 164-172 (1999).

Morgan, R. M., Patterson, M. J., Nimmo, M. A. Acute effects of dehydration on sweat composition in men during prolonged exercise in the heat. Acta Physiol. Scand. 182, 37-43 (2004).

Young, V.; Garza, C. Dietary Reference Intakes for Calcium, Phosphorus, Magnesium, Vitamin D and Fluoride. Standing Committee on the Scientific Evaluation of Dietary Reference Intakes, Food and Nutritional Board, Institute of Medicine; National Academy Press: Washington, D.C., 1997.

Robertson, W. G.; Marshall, R. W.; Bowers, G. N. Ionized Calcium in Body Fluids. CRC Crit. Rev. Clin. Lab. Sci. 1981, 15, 85-125.

Relman, R. S. Metabolic Consequences of Acid-Base Disorders.Kidney Int. 1972, 1, 347-359.

Maalouf, N. M.; Cameron, M. A.; Moe, O. W.; Sakhaee, K. Metabolic Basis for Low Urine pH in Type 2 Diabetes. Clin. J. Am. Soc. Nephrol. 2010, 5, 1277-1281.

Schmid-Wendtner, M. H.; Korting, H. C. The pH of the Skin Surface and its Impact on the Barrier Function. Skin Pharmacol. Physiol. 2006, 19, 296-302.

Bandodkar, A. J.; Jeerapan, I.; Wang, J. Wearable Chemical Sensors: Present Challenges and Future Prospects. ACS Sens. 2016, 1, 464-482.

Kim, D. H.; Lu, N. S.; Ma, R.; Kim, Y. S.; Kim, R. H.; Wang, S. D.; Wu, J.; Won, S. M.; Tao, H.; Islam, A.; Yu, K. J.; Kim, T. I.; Chowdhury, R.; Ying, M.; Xu, L. Z.; Li, M.; Chung, H. J.; Keum, H.; McCormick, M.; Liu, P.; Zhang, Y. W.; Omenetto, F. G.; Huang, Y. G.; Coleman, T.; Rogers, J. Epidermal Electronics. Science 2011, 333, 838-843.

Wang, C.; Hwang, D.; Yu, Z.; Takei, K.; Park, J.; Chen, T.; Ma, B.; Javey, A. User-Interactive Electronic Skin for Instantaneous Pressure Visualization. Nat. Mater. 2013, 12, 899-904.

Schwartz, G.; Tee, B. C. K.; Mei, J.; Appleton, A. L.; Kim, D. H.; Wang, H.; Bao, Z. Flexible Polymer Transistors with High Pressure Sensitivity for Application in Electronic Skin and Health Monitoring. Nat. Commun. 2013, 4, 1859.

Xu, S.; Zhang, Y.; Jia, L.; Mathewson, K. E.; Jang, K.; Kim, J.; Fu, H.; Huang, X.; Chava, P.; Wang, R.; Bhole, S.; Wang, L.; Na, Y. J.; Guan, Y.; Flavin, M.; Han, Z.; Huang, Y.; Rogers, J. A. Soft Microfluidic Assemblies of Sensors, Circuits, and Radios for the Skin. Science 2014, 344, 70-74.

Kaltenbrunner, M.; Sekitani, T.; Reeder, J.; Yokota, T.; Kuribara, K.; Tokuhara, T.; Drack, M.; Schwodiauer, R.; Graz, I.; Bauer Gogonea, S.; Bauer, S.; Someya, T. An Ultra-Lightweight Design for Imperceptible Plastic Electronics. Nature 2013, 499, 458-463.

Tee, B. C. K.; Chortos, A.; Berndt, A.; Nguyen, A. K.; Tom, A.; McGuire, A.; Lin, Z. C.; Tien, K.; Bae, W.; Wang, H.; Mei, P.; Chou, H.; Cui, B.; Deisseroth, K.; Ng, T. N.; Bao, Z. A Skin-Inspired Organic

Takei, K.; Honda, W.; Harada, S.; Arie, T.; Akita, S. Flexible and Wearable Human-Interactive Health-Monitoring Devices. Adv. Health-care Mater. 2015, 4, 487-500.

Chen, K.; Gao, W.; Emaminejad, S.; Kiriya, D.; Ota, H.; Nyein H. Y. Y.; Takei, K.; Javey, A. Printed Carbon Nanotube Electronics and Sensor Systems. Adv. Mater. 2016, 28, 4397-4414.

Huang, X.; Liu, Y. H.; Chen, K. L.; Shin, W. J.; Lu, C. J.; Kong, G. W.; Patnaik, D.; Lee, S. H.; Cortes, J. F.; Rogers, J. A. Stretchable, Wireless Sensors and Functional Substrates for Epidermal Characterization of Sweat. Small 2014, 10, 3083-3090.

Curto, V. F.; Fay, C.; Coyle, S.; Byrne, R.; O'Toole, C.; Barry, C.; Hughes, S.; Moyna, N.; Diamond, D.; Benito-Lopez, F. Real-time Sweat pH Monitoring Based on a Wearable Chemical Barcode Micro-Fluidic Platform Incorporating Ionic Liquids. Sens. Actuators, B 2012, 171-172, 1327-1334.

Sonner, Z.; Wilder, E.; Heikenfeld, J.; Kasting, G.; Beyette, F.; Swaile, D.; Sherman, F.; Joyce, J.; Hagen, J.; Kelley-Loughnane, N.; Naik, R. The Microfluidics of the Eccrine Sweat Gland, Including Biomarker Partitioning, Transport, and Biosensing Implications. Biomicrofluidics 2015, 9, 031301.

Bandodkar, A. J.; Hung, V. W. S.; Jia, W.; Valdes-Ramirez, G.; Windmiller, J. R.; Martinez, A. G.; Ramirez, J.; Chan, G.; Kerman, K.; Wang, J. Tattoo-based Potentiometric Ion-Selective Sensors for Epidermal pH Monitoring. Analyst 2013, 138, 123-128.

Bandodkar, A. J.; Molinnus, D.; Mirza, O.; Guinovart, T.; Windmiller, J. R.; Valdes-Ramirez, G.; Andrade, F. J.; Schoning, M. J.; Wang, J. Epidermal Tattoo Potentiometric Sodium Sensors with Wireless Signal Transduction for Continuous Noninvasive Sweat Monitoring. Biosens. Bioelectron. 2014, 54, 603-609.

Jia, W.; Bandodkar, A. J.; Valdes-Ramirez, G.; Windmiller, J. R.; Yang, Z.; Ramirez, J.; Chan, G.; Wang, J. Electrochemical Tattoo Biosensors for Real-Time Noninvasive Lactate Monitoring in Human Perspiration. Anal. Chem. 2013, 85, 6553-6560.

Rose, D. P.; Ratterman, M.; Griffin, D. K.; Hou, L.; Kelley-Loughnane, N.; Naik, R. K.; Hagen, J. A.; Papautsky, I.; Heikenfeld, J. Adhesive RFID Sensor Patch for Monitoring of Sweat Electrolytes. IEEE Trans. Biomed. Eng. 2014, 62, 1457-1465.

Schazmann, B.; Morris, D.; Slater, C.; Beirne, S.; Fay, C.; Reuveny, R.; Moyna, N.; Diamond, D. A Wearable Electrochemical Sensor for the Real-Time Measurement of Sweat Sodium Concentration. Anal. Methods 2010, 2, 342-348.

Kim, J.; Araujo, W. R.; Samek, I. A.; Bandodkar, A. J.; Jia, W.; Brunetti, B.; Paixao, T. R. L. C.; Wang, J. Wearable Temporary Tattoo Sensor for Real-time Trace Metal Monitoring in Human Sweat. Electrochem. Commun. 2015, 51, 41-45.

Gao, W.; Nyein, H. Y. Y.; Shahpar, Z.; Fahad, H. M.; Chen, K.; Emaminejad, S.; Gao, Y.; Tai, L.-C.; Ota, H.; Wu, E.; Bullock, J.; Zeng, Y.; Lien, D.-H.; Javey, A. Wearable Microsensor Array for Multiplexed Heavy Metal Monitoring of Body Fluids. ACS Sens. 2016, DOI: 10.1021/acssensors.6b00287.

Lee, H.; Choi, T. K.; Lee, Y. B.; Cho, H. R.; Ghaffari, R.; Wang, L.; Choi, H. J.; Chung, T. D.; Lu, N.; Hyeon, T.; Choi, S. H.; Kim, D. H. A Graphene-Based Electrochemical Device with Thermoresponsive Microneedles for Diabetes Monitoring and Therapy. Nat. Nanotechnol. 2016, 11, 566-572.

Shortreed, M.; Kopelman, R.; Kuhn, M.; Hoyland, B. Fluorescent Fiber-Optic Calcium Sensor for Physiological Measurements. Anal. Chem. 1996, 68, 1414-1418.

Shalom, S.; Strinkovski, A.; Peleg, G.; Druckmann, S.; Krauss, A.; Lewis, A.; Linial, M.; Ottolenghi, M. An Optical Submicrometer Calcium Sensor with Conductance Sensing Capability. Anal. Biochem. 1997, 244, 256-259.

Bülmann, P.; Pretsch, E.; Bakker, E. Carrier-Based Ion-Selective Electrodes and Bulk Optodes. 2. Ionophores for Potentiometric and Optical Sensors. Chem. Rev. 1998, 98, 1593-1687.

Prestipino, G.; Falugi, C.; Falchetto, R.; Gazzotti, P. The Ionophore ETH 129 as Ca Translocator in Artificial and Natural Membranes. Anal. Biochem. 1993, 210, 119-122.

Bedlechowicz, I.; Sokalski, T.; Lewenstamb, A.; Majzurawska, M. Calcium Ion-Selective Electrodes under Galvanostatic Current Control. Sens. Actuators, B 2005, 108, 836-839.

Lindfors, T.; Ivaska, A. Calcium-Selective Electrode Based on Polyaniline Functionalized with Bis[4-(1,1,3,3-tetramethylbutyl)-phenyl]phosphate. Anal. Chim. Acta 2001, 437, 171-182.

Gao, W.; Emaminejad, S.; Nyein, H. Y. Y.; Challa, S.; Chen, K.; Peck, A.; Fahad, H. M.; Ota, H.; Shiraki, H.; Kiriya, D.; Lien, D.-H.; Brooks, G. A.; Davis, R. W.; Javey, A. Fully Integrated Wearable Sensor Arrays for Multiplexed In Situ Perspiration Analysis. Nature 2016, 529, 509-514.

Tiwari, A.; Patra, H. K.; Turner, A. P. F. Advanced Bioelectronic Materials; Scrivener: Beverly, Mass., 2015; p 107.

Guinovart, T.; Crespo, G. A.; Rius, F. X.; Andrade, F. J. A Reference Electrode Based on Polyvinyl Butyral (PVB) Polymer for Decentralized Chemical Measurements. Anal. Chim. Acta 2014, 821, 72-80.

Baker, L. B.; Stofan, J. R.; Lukaski, H. C.; Horswill, C. A. Exercise-Induced Trace Mineral Element Concentration in Regional versus Whole-Body Wash-Down Sweat. Int. J. Sport Nutr. Exercise Metab. 2011, 21, 233-239.

Katz, S.; Schoni, M. H.; Bridges, M. A. The Calcium Hypothesis of Cystic Fibrosis. Cell Calcium 1984, 5, 421-440.

Verde, T.; Shephard, R. J.; Corey, P.; Moore, R. Sweat Composition in Exercise and in Heat. J. Appl. Physiol. 1982, 53, 1540-1545.

Shirreffs, S. M.; Maughan, R. J. Whole Body Sweat Collection in Humans: An Improved Method with Preliminary Data on Electrolyte Content. J. Appl. Physiol. 1997, 82, 336-341.

Ansaldi, A.; Epstein, S. I. Calcium Ion-Selective Electrode in which a Membrane Contacts Graphite directly. Anal. Chem. 1973, 45, 595-596.

Kent, P.; Bunce, S. C.; Bailey, R. A.; Aikens, D. A. Interference by Polyamines in the Measurement of Magnesium Ion at Physiological pH with the Divalent Cation-Selective Electrode. Anal. Biochem. 1974, 62, 75-80.

Matousek, J. L.; Campbell, K. L. A Comparative Review of Cutaneous pH. Adv. Vet. Dermatol. 2002, 13, 293-300.

Suzuki, H.; Shiroishi, H. Microfabricated Liquid Junction Ag/AgCl Reference Electrode and Its Application to a One-Chip Potentiometric Sensor. Anal. Chem. 1999, 71, 5069-5075.

Sprigle, S.; Linden, M.; McKenna, D.; Davis, K.; Riordan, B. Clinical Skin Temperature Measurement to Predict Incipient Pressure Ulcers. Adv. Skin Wound Care 2001, 14, 133-137.

Kim, D. H.; Lu, N. S.; Ma, R.; Kim, Y. S.; Kim, R. H.; Wang, S. D.; Wu, J.; Won, S. M.; Tao, H.; Islam, A.; Yu, K. J.; Kim, T. I.; Chowdhury, R.; Ying, M.; Xu, L. Z.; Li, M.; Chung, H. J.; Keum, H.; McCormick, M.; Liu, P.; Zhang, Y. W.; Omenetto, F. G.; Huang, Y. G.; Coleman, T.; Rogers, J. A. Epidermal Electronics. Science 2011, 333, 838-843.

Bandodkar, A. J.; Wang, J. Non-invasive Wearable Electro-chemical Sensors: A Review. Trends Biotechnol. 2014, 32, 363-371.

Schwartz, G.; Tee, B. C. K.; Mei, J.; Appleton, A. L.; Kim, D. H.; Wang, H.; Bao, Z. Flexible Polymer Transistors with High Pressure Sensitivity for Application in Electronic Skin and Health Monitoring. Nat. Commun. 2013, 4, 1859.

Kaltenbrunner, M.; Sekitani, T.; Reeder, J.; Yokota, T.; Kuribara, K.; Tokuhara, T.; Drack, M.; Schwodiauer, R.; Graz, I.; Bauer Gogonea, S.; Bauer, S.; Someya, T. An Ultra-Lightweight Design for Imperceptible Plastic Electronics. Nature 2013, 499, 458-463.

Takei, K.; Takahashi, T.; Ho, J. C.; Ko, H.; Gillies, A. G.; Leu, P. W.; Fearing, R. S.; Javey, A. Nanowire Active-Matrix Circuitry for Low-Voltage Macroscale Artificial Skin. Nat. Mater. 2010, 9, 821-826.

Hammock, M. L.; Chortos, A.; Tee, B. C. K.; Tok, J. B. H.; Bao, Z. The Evolution of Electronic Skin (e-skin): A Brief History, Design Considerations, and Recent Progress. Adv. Mater. 2013, 25, 5997-6037.

Wang, C.; Hwang, D.; Yu, Z.; Takei, K.; Park, J.; Chen, T.; Ma, B.; Javey, A. User-Interactive Electronic Skin for Instantaneous Pressure Visualization. Nat. Mater. 2013, 12, 899-904.

Xu, S.; Zhang, Y.; Jia, L.; Mathewson, K. E.; Jang, K.; Kim, J.; Fu, H.; Huang, X.; Chava, P.; Wang, R.; Bhole, S.; Wang, L.; Na, Y. J.; Guan, Y.; Flavin, M.; Han, Z.; Huang, Y.; Rogers, J. A. Soft Microfluidic Assemblies of Sensors, Circuits, and Radios for the Skin. Science 2014, 344, 70-74.

Takei, K.; Honda, W.; Harada, S.; Arie, T.; Akita, S. Flexible and Wearable Human-Interactive Health-Monitoring Devices. Adv. Health-care Mater. 2015, 4, 487-500.

Fan, F. R.; Lin, L.; Zhu, G.; Wu, W. Z.; Zhang, R.; Wang, Z. L. Transparent Triboelectric Nanogenerators and Self-Powered Pressure Sensors Based on Micropatterned Plastic Films. Nano Lett. 2012, 12, 3109-3114.

Chen, K.; Gao, W.; Emaminejad, S.; Kiriya, D.; Ota, H.; Nyein, H. Y. Y.; Takei, K.; Javey, A. Printed Carbon Nanotube Electronics and Sensor Systems. Adv. Mater. 2016, DOI: 10.1002/adma.201504958.

Ota, H.; Emaminejad, S.; Gao, Y.; Zhao, A.; Wu, E.; Challa, S.; Chen, K.; Fahad, H. M.; Jha, A. K.; Kiriya, D.; Gao, W.; Shiraki, H.; Morioka, K.; Ferguson, A. R.; Healy, K. E.; Davis, R. W.; Javey, A. Application of 3D Printing for Smart Objects with Embedded Electronic Sensors and Systems. Adv. Mater. Technol. 2016, 1, 1600013.

Schazmann, B.; Morris, D.; Slater, C.; Beirne, S.; Fay, C.; Reuveny, R.; Moyna, N.; Diamond, D. A Wearable Electrochemical Sensor for the Real-Time Measurement of Sweat Sodium Concentration. Anal. Methods 2010, 2, 342-348.

Jia, W.; Bandodkar, A. J.; Valdes-Ramirez, G.; Windmiller, J. R.; Yang, Z.; Ramirez, J.; Chan, G.; Wang, J. Electrochemical Tattoo Biosensors for Real-Time Noninvasive Lactate Monitoring in Human Perspiration. Anal. Chem. 2013, 85, 6553-6560.

Bandodkar, A. J.; Molinnus, D.; Mirza, O.; Guinovart, T.; Windmiller, J. R.; Valdes-Ramirez, G.; Andrade, F. J.; Schoning, M. J.; Wang, J. Epidermal Tattoo Potentiometric Sodium Sensors with Wireless Signal Transduction for Continuous Non-Invasive Sweat Monitoring. Biosens. Bioelectron. 2014, 54, 603-609.

Sonner, Z.; Wilder, E.; Heikenfeld, J.; Kasting, G.; Beyette, F.; Swaile, D.; Sherman, F.; Joyce, J.; Hagen, J.; Kelley-Loughnane, N.; Naik, R. The Microfluidics of the Eccrine Sweat Gland, Including Biomarker Partitioning, Transport, and Biosensing Implications. Biomicrofluidics 2015, 9, 031301.

Gonzalo-Ruiz, J.; Mas, R.; de Haro, C.; Cabruja, E.; Camero, R.; Alonso-Lomillo, M. A.; Muñoz, F. J. Integration of Biosensors and Drug Delivery Technologies for Early Detection and Chronic Management of Illness. Biosens. Bioelectron. 2009, 24, 1788-1791.

Huang, X.; Liu, Y. H.; Chen, K. L.; Shin, W. J.; Lu, C. J.; Kong, G. W.; Patnaik, D.; Lee, S. H.; Cortes, J. F.; Rogers, J. A. Stretchable, Wireless Sensors and Functional Substrates for Epidermal Characterization of Sweat. Small 2014, 10, 3083-3090.

Kim, J.; Imani, S.; de Araujo, W. R.; Warchall, J.; Valdes-Ramírez, G.; Paixao, T. R. L. C.; Mercier, P.; Wang, J. Wearable Salivary Uric Acid Mouthguard Biosensor with Integrated Wireless Electronics. Biosens. Bioelectron. 2015, 74, 1061-1068.

Yao, H.; Shum, A. J.; Cowan, M.; Landesmaki, I.; Parviz, B. A. A Contact Lens with Embedded Sensor for Monitoring Tear Glucose Level. Biosens. Bioelectron. 2011, 26, 3290-3296.

Chu, M. X.; Kudo, H.; Shirai, T.; Miyajima, K.; Saito, H.; Morimoto, N.; Yano, K.; Iwasaki, Y.; Akiyoshi, K.; Mitsubayashi, K. A Soft and Flexible Biosensor Using a Phospholipid Polymer for Continuous Glucose Monitoring. Biomed. Microdevices 2009, 11, 837-842.

Bandodkar, A. J.; Jia, W.; Yardimci, C.; Wang, X.; Ramirez, J.; Wang, J. Tattoo-Based Noninvasive Glucose Monitoring: A Proof-of-Concept Study. Anal. Chem. 2015, 87, 394-398.

Gao, W.; Emaminejad, S.; Nyein, H. Y. Y.; Challa, S.; Chen, K.; Peck, A.; Fahad, H. M.; Ota, H.; Shiraki, H.; Kiriya, D.; Lien, D.-H.; Brooks, G. A.; Davis, R. W.; Javey, A. Fully Integrated Wearable Sensor Arrays for Multiplexed In Situ Perspiration Analysis. Nature 2016, 529, 509-514.

Lee, H.; Choi, T. K.; Lee, Y. B.; Cho, H. R.; Ghaffari, R.; Wang, L.; Choi, H. J.; Chung, T. D.; Lu, N.; Hyeon, T.; Choi, S. H.; Kim, D. H. A Graphene-B ased Electrochemical Device with Thermoresponsive Microneedles for Diabetes Monitoring and Therapy. Nat. Nanotechnol. 2016, DOI: 10.1038/nnano.2016.38.

Cohn, J. R.; Emmett, E. A. The Excretion of Trace Metals in Human Sweat. Ann. Clin. Lab Sci. 1978, 8, 270-275.

Sears, M. E.; Kerr, K. J.; Bray, R. I. Arsenic, Cadmium, Lead, and Mercury in Sweat: A Systematic Review. J. Environ. Public Health 2012, 2012, 184745.

Mudgal, V.; Madaan, N.; Mudgal, A.; Singh, R. B.; Mishra, S. Effect of Toxic Metals on Human Health. Open Nutraceuticals J. 2010, 3, 94-99.

Fraga, C. G. Relevance, Essentiality and Toxicity of Trace Elements in Human Health. Mol. Aspects Med. 2005, 26, 235.

Schaefer, M.; Schellenberg, M.; Merle, U.; Weiss, K. H.; Stremmel, W. Wilson Protein Expression, Copper Excretion and Sweat Production in Sweat Glands of Wilson Disease Patients and Controls. BMC Gastroenterol. 2008, 8, 29.

Hohnadel, D. C.; Sunderman, F. W., Jr.; Nechay, M. W.; McNeely, M. D. Atomic Absorption Spectrometry of Nickel, Copper, Zinc, and Lead in Sweat Collected from Healthy Subjects during Sauna Bathing. Clin. Chem. 1973, 19, 1288-1292.

Munro, S.; Ebdon, L.; McWeeny, D. J. J. Application of Inductively Coupled Plasma Mass Spectrometry (ICP-MS) for Trace Metal Determination in Foods. J. Anal. At. Spectrom. 1986, 1, 211-219.

Wang, J. Stripping Analysis: Principles, Instrumentation, and Applications; VCH Publishers: Deerfield Beach, Fla., 1985.

Stauber, J. L.; Florence, T. M. The Determination of Trace Metals in Sweat by Anodic Stripping Voltammetry. Sci. Total Environ. 1987, 60, 263-271.

de Souza, A. P. R.; Lima, A. S.; Salles, M. O.; Nascimento, A. N.; Bertotti, M. The Use of a Gold Disc Microelectrode for the Determination of Copper in Human Sweat. Talanta 2010, 83, 167-170.

Crew, A.; Cowell, D. C.; Hart, J. P. Development of an Anodic Stripping Voltammetric Assay, Using a Disposable Mercury-Free Screen-Printed Carbon Electrode, for the Determination of Zinc in Human Sweat. Talanta 2008, 75, 1221-1226.

Hutton, E. A.; Ogorevc, B.; Hocevar, S. B.; Smyth, M. R. Bismuth Film Microelectrode for Direct Voltammetric Measurement of Trace Cobalt and Nickel in Some Simulated and Real Body Fluid Samples. Anal. Chim. Acta 2006, 557, 57-63.

Kim, J.; de Araujo, W. R.; Samek, I. A.; Bandodkar, A. J.; Jia, W.; Brunetti, B.; Paixao, T. R.L.C.; Wang, J. Wearable Temporary Tattoo Sensor for Real-Time Trace Metal Monitoring in Human Sweat. Electrochem. Commun. 2015, 51, 41-45.

Wang, J. Stripping Analysis at Bismuth Electrodes: A Review. Electroanalysis 2005, 17, 1341-1346.

Bonfil, Y.; Brand, M.; Kirowa-Eisner, E. Characteristics of Subtractive Anodic Stripping Voltammetry of Pb and Cd at Silver and Gold Electrodes. Anal. Chim. Acta 2002, 464, 99-114.

Wang, J.; Deo, R. P.; Thongngamdee, S.; Ogorevc, B. Effect of Surface-Active Compounds on the Stripping Voltammetric Response of Bismuth Film Electrodes. Electroanalysis 2001, 13, 1153-1156.

Pei, X.; Kang, W.; Yue, W.; Bange, A.; Heineman, W. R.; Papautsky, I. Disposable Copper-Based Electrochemical Sensor for Anodic Stripping Voltammetry. Anal. Chem. 2014, 86, 4893-4900.

Soleymani, L.; Fang, Z.; Lam, B.; Xiaomin Bin, X.; Vasilyeva, E. Ross, A. J.; Sargent, E. H.; Kelley, S. O. Hierarchical Nanotextured Microelectrodes Overcome the Molecular Transport Barrier to Achieve Rapid, Direct Bacterial Detection. ACS Nano 2011, 5, 3360-3366.

Omokhodion, F. O.; Howard, J. M. Lead in Sweat and its Relationship to Salivary and Urinary Levels in Normal Healthy Subjects. Sci. Total Environ. 1991, 103, 123-128.

The methods and processes described herein may have fewer or additional steps or states and the steps or states may be performed in a different order. Not all steps or states need to be reached. The methods and processes described herein may be embodied in, and fully or partially automated via, software code modules executed by one or more general purpose computers, microcontrollers, and/or other processing devices. The code modules may be stored in any type of computer-readable medium or other computer storage device. Some or all of the methods may alternatively be embodied in whole or in part in specialized computer hardware. The systems described herein may optionally include displays, user input devices (e.g., touchscreen, keyboard, mouse, voice recognition, etc.), network interfaces, etc.

The results of the disclosed methods may be stored in any type of computer data repository, such as relational databases and flat file systems that use volatile and/or non-volatile memory (e.g., magnetic disk storage, optical storage, EEPROM and/or solid state RAM).

The various illustrative logical blocks, modules, routines, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

While the phrase “click” may be used with respect to a user selecting a control, menu selection, or the like, other user inputs may be used, such as voice commands, text entry, gestures, etc. User inputs may, by way of example, be provided via an interface, such as via text fields, wherein a user enters text, and/or via a menu selection (e.g., a drop down menu, a list or other arrangement via which the user can check via a check box or otherwise make a selection or selections, a group of individually selectable icons, etc.). When the user provides an input or activates a control, a corresponding computing system may perform the corresponding operation. Some or all of the data, inputs and instructions provided by a user may optionally be stored in a system data store (e.g., a database), from which the system may access and retrieve such data, inputs, and instructions. The notifications and user interfaces described herein may be provided via a Web page, a dedicated or non-dedicated phone application, computer application, a short messaging service message (e.g., SMS, MMS, etc.), instant messaging, email, push notification, audibly, and/or otherwise.

The user terminals described herein may be in the form of a mobile communication device (e.g., a cell phone), laptop, tablet computer, interactive television, game console, media streaming device, head-wearable display, networked watch, etc. The user terminals may optionally include displays, user input devices (e.g., touchscreen, keyboard, mouse, voice recognition, etc.), network interfaces, etc.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A wearable biometric monitoring system comprising:
a first sensor configured to sense a first sweat analyte;
a second sensor configured to sense a second sweat analyte at substantially the same time as the first sensor is measuring the first sweat analyte;
a signal conditioner coupled to the first sensor and the second sensor, the signal conditioner configured receive and condition sensor signals from the first sensor and the second sensor, the signal conditioner comprising one or more amplifiers and one or more filters; and
an interface configured to transmit information corresponding to the conditioned sensor signals to a remote computing device.

2. The wearable biometric monitoring system as defined in claim 1, further comprising a temperature sensor configured to measure skin temperature of a wearer of the biometric system.

3. The wearable biometric monitoring system as defined in claim 1, further comprising a temperature sensor configured to measure skin temperature of a wearer of the biometric system, wherein the wearable biometric monitoring system is configured to compensate, in real-time, one or more sensor readings.

4. The wearable biometric monitoring system as defined in claim 1, further comprising a temperature sensor configured to measure skin temperature of a wearer of the biometric system, wherein the wearable biometric monitoring system is configured to linearly compensate, in real-time, one or more sensor readings.

5. The wearable biometric monitoring system as defined in claim 1, further comprising a temperature sensor configured to determine skin temperature of a wearer of the wearable biometric monitoring system, wherein a processor or circuit is configured to calibrate a response of at least one of the first sensor or the second sensor based on the determined skin temperature.

6. The wearable biometric monitoring system as defined in claim 1, further comprising a processor or circuit configured to calibrate a response of at least one of the first sensor or the second sensor based on at least one of a pH of a wearer's sweat or temperature.

7. The wearable biometric monitoring system as defined in claim 1, further comprising a third sensor configured to measure a third sweat analyte and a fourth sensor configured to measure a fourth sweat analyte at substantially the same time as the first sensor is measuring the first sweat analyte.

8. The wearable biometric monitoring system as defined in claim 7, wherein the first sweat analyte comprises glucose, the second sweat analyte comprises lactate, the third sweat analyte comprises sodium, and the fourth sweat analyte comprises potassium.

9. The wearable biometric monitoring system as defined in claim 7, wherein the first sweat analyte comprises a first metabolite, the second sweat analyte comprises a second metabolite different from the first metabolite, the third sweat analyte comprises a first electrolyte, and the fourth sweat analyte comprises a second electrolyte different from the first electrolyte.

10. The wearable biometric monitoring system as defined in claim 7, wherein the first sweat analyte comprises a metabolite, the second sweat analyte comprises an electrolyte, the third sweat analyte comprises a protein, and the fourth sweat analyte comprises a heavy metal.

11. The wearable biometric monitoring system as defined in claim 1, wherein the first sweat analyte comprises a metabolite, an electrolyte, a protein, or a heavy metal.

12. The wearable biometric monitoring system as defined in claim 1, comprising one or more sensors comprising glucose oxidase (GOx) and/or lactate oxidase (LOx) immobilized within a chitosan film.

13. The wearable biometric monitoring system as defined in claim 1, comprising an Ag/AgCl shared reference electrode for the first sensor and the seconds sensor.

14. The wearable biometric monitoring system as defined in claim 1, comprising one or more sensors configured to sense pH and Ca²⁺.

15. The wearable biometric monitoring system as defined in claim 1, comprising a Ca²⁺ sensor having a polystyrene sulfonate layer, an Au layer, and a PET layer.

16. The wearable biometric monitoring system as defined in claim 1, comprising a pH sensor having a PVB layer, an Au layer, and a PET layer.

17. The wearable biometric monitoring system as defined in claim 1, comprising one or more sensors configured to sense Zn, Cd, Pb, Cu, and/or Hg.

18. The wearable biometric monitoring system as defined in claim 1, comprising a heavy metal sensor having an Au or Bi layer and a PET layer.

19. The wearable biometric monitoring system as defined in claim 1, wherein the first sweat analyte comprises a metabolite and the second sweat analyte comprises an electrolyte.

20. The wearable biometric monitoring system as defined in claim 1, wherein the first sweat analyte comprises at least one of a metabolite or a protein, and the second sweat analyte comprises at least one of an electrolyte or a heavy metal.

21. The wearable biometric monitoring system as defined in claim 1, wherein the first sweat analyte comprises one of a metabolite, a protein, an electrolyte, or a heavy metal, and wherein the second sweat analyte comprises a different one of a metabolite, a protein, an electrolyte, or a heavy metal.

22. The wearable biometric monitoring system as defined in claim 1, wherein each sensor of a plurality of sensors has its own dedicated signal conditioning path, wherein the plurality of sensors includes the first sensor and the second sensor.

23. The wearable biometric monitoring system as defined in claim 1, further comprising a multiplexer configured to selectively couple the signal conditioner to one of a plurality of sensors, wherein the plurality of sensors includes the first sensor and the second sensor.

24. The wearable biometric monitoring system as defined in claim 1, wherein the first sensor and the second sensor are plastic-based sensors and the signal conditioner, the first and second sensors, the interface, and a battery, are mounted on a flexible substrate.

25. The wearable biometric monitoring system as defined in claim 1, wherein the first sensor and the second sensor are plastic-based sensors and the signal conditioner, the first and second sensors, and the interface are mounted on a mechanically flexible polyethylene terephthalate (PET) substrate.

26. The wearable biometric monitoring system as defined in claim 1, wherein the first sensor and the second sensor are plastic-based sensors and the signal conditioner, the first and second sensors, and the interface are mounted on together on a single flexible circuit board.

27. The wearable biometric monitoring system as defined in claim 1, wherein the first and second sensors are mounted on a flexible substrate configured to be worn around a wrist, arm, ankle, or leg of a wearer.

28. The wearable biometric monitoring system as defined in claim 1, wherein the first and second sensors are mounted on a flexible substrate configured to be worn so as to be in contact with a head or chest of a wearer.

29. The wearable biometric monitoring system as defined in claim 1, wherein the wearable biometric monitoring system is configured to detect when a wearer is dehydrated based at least in part on the sensor signals.

30. The wearable biometric monitoring system as defined in claim 1, wherein the wearable biometric monitoring system is configured to detect a likelihood or presence of hyponatremia, hypokalemia, muscle cramps, ischemia, and/or pressure ulcers of a wearer based at least in part on the sensor signals.

31. The wearable biometric monitoring system as defined in claim 1, wherein the interface comprises a wireless interface configured to communicate with an application hosted on a data aggregation device configured with a display.

32. The wearable biometric monitoring system as defined in claim 1, wherein the interface comprises a wireless interface configured to communicate with a remote system.

33. The wearable biometric monitoring system as defined in claim 1, wherein the first and second sensors are configured to autonomously generate current signals proportional to an amount of a metabolite between a working electrode and a reference electrode.

34. The wearable biometric monitoring system as defined in claim 1, wherein the first sensor comprises an amperometric glucose sensor that utilizes glucose oxidase (GOx) and the second sensor comprises a lactate sensor that utilizes lactate oxidase (LOx), the GOx and the LOx immobilized within a chitosan film on working electrodes.

35. The wearable biometric monitoring system as defined in claim 1, wherein the signal conditioner and interface comprise silicon integrated circuits.

36. The wearable biometric monitoring system as defined in claim 1, further comprising a display configured to display analysis of sensor signals.

37. The wearable biometric monitoring system as defined in claim 1, further comprising a wireless interface configured to communicate sensor readings to a mobile device, the mobile device hosting an application configured to display information related to the sensor readings.

38. The wearable biometric monitoring system as defined in claim 1, further comprising a wireless interface configured to communicate sensor readings to a mobile device, the mobile device hosting an application configured to upload the sensor readings to a cloud-based system and/or to transmit the sensor readings to one or more recipients.

39. The wearable biometric monitoring system as defined in claim 1, further comprising a wireless interface configured to communicate sensor readings to a mobile device, the mobile device hosting an application configured to detect a user condition and to generate a corresponding alert.

40. A wearable biometric monitoring system comprising:
a flexible substrate;
a plurality of sweat analyte sensors affixed to the flexible substrate, the plurality of sweat analyte sensors configured to sense a plurality of different sweat analytes of a wearer at substantially the same time, the plurality of sweat analyte sensors comprising at least a first sweat analyte sensor configured to sense a metabolite and a second sweat analyte configured to sense an electrolyte;
a temperature sensor configured to measure skin temperature of the wearer;
a signal conditioner affixed to the flexible substrate, the signal conditioner coupled to the plurality of sweat analyte sensors, the signal conditioner configured receive and condition sensor signals from the plurality of sweat analyte sensors, the signal conditioner comprising one or more amplifiers and one or more filters;
an analog and digital converter configured to convert the conditioned sensor signals from an analog domain to a digital domain, and a digital processor configured to digitally process the converted sensor signals in the digital domain, the analog and digital converter and the digital processor affixed to the flexible substrate;
an interface configured to transmit information corresponding to the conditioned sensor signals to a remote computing device, the interface affixed to the flexible substrate; and
a battery configured to power at least portions of the wearable biometric monitoring system.

41. The wearable biometric monitoring system as defined in claim 40, the wearable biometric monitoring system configured to compensate, in real-time, one or more sensor readings.

42. The wearable biometric monitoring system as defined in claim 40, wherein the plurality of sweat analyte sensors comprises a glucose sensor, a lactate sensor, a sodium sensor, and a potassium sensor.

43. The wearable biometric monitoring system as defined in claim 40, wherein the plurality of sweat analyte sensors comprises a protein sensor and/or a heavy metal sensor.

44. The wearable biometric monitoring system as defined in claim 40, wherein the plurality of sweat analyte sensors comprises a pH sensor and/or a Ca²⁺ sensor.

45. The wearable biometric monitoring system as defined in claim 40, comprising one or more sensors configured to sense Zn, Cd, Pb, Cu, and/or Hg.

46. The wearable biometric monitoring system as defined in claim 40, wherein the plurality of sweat analyte sensors comprises plastic-based sensors.

47. The wearable biometric monitoring system as defined in claim 40, wherein the flexible substrate comprises a flexible polyethylene terephthalate (PET) substrate.

48. The wearable biometric monitoring system as defined in claim 40, wherein the wearable biometric monitoring system is configured to be bendable into a band shape.

49. The wearable biometric monitoring system as defined in claim 40, wherein the wearable biometric monitoring system is configured to be worn around a wrist, an arm, or a head of a wearer.

50. The wearable biometric monitoring system as defined in claim 40, wherein the wearable biometric monitoring system is configured to detect when a wearer is dehydrated based at least in part on the sensor signals.

51. The wearable biometric monitoring system as defined in claim 40, wherein the wearable biometric monitoring system is configured to detect a likelihood or presence of hyponatremia, hypokalemia, muscle cramps, ischemia, and/or pressure ulcers of a wearer based at least in part on the sensor signals.

52. The wearable biometric monitoring system as defined in claim 40, wherein the interface comprises a wireless interface configured to communicate with an application hosted on a data aggregation device configured with a display.

53. The wearable biometric monitoring system as defined in claim 40, wherein the interface comprises a wireless interface configured to wirelessly communicate with another system.

54. The wearable biometric monitoring system as defined in claim 40, wherein at least a first of the plurality of sweat analyte sensors comprises sensors is configured to autonomously generate current signals proportional to an amount of a first metabolite between a working electrode and a reference electrode of the first sweat analyte sensor.

55. The wearable biometric monitoring system as defined in claim 40, wherein the plurality of sweat analyte sensors comprises an amperometric glucose sensor that utilizes glucose oxidase (GOx) and a lactate sensor that utilizes lactate oxidase (LOx), the GOx and the LOx immobilized within a chitosan film on working electrodes.

56. The wearable biometric monitoring system as defined in claim 40, wherein the signal conditioner and interface comprise silicon integrated circuits.

57. The wearable biometric monitoring system as defined in claim 40, further comprising a display configured to display an analysis of sensor signals.

58. The wearable biometric monitoring system as defined in claim 40, further comprising a microprocessor configured to compensate, in real-time, a response of at least one of the first sweat analyte sensor or the second sweat analyte sensor based on the measured skin temperature.

59. The wearable biometric monitoring system as defined in claim 40, further comprising a processor or circuit configured to calibrate a response of at least one of the first sweat analyte sensor or the second sweat analyte sensor based on the measured skin temperature.

60. The wearable biometric monitoring system as defined in claim 40, further comprising a processor or circuit configured to calibrate a response of at least one of the first sweat analyte sensor or the second sweat analyte sensor based on at least one of a pH of a wearer's sweat or the measured skin temperature.

61. The wearable biometric monitoring system as defined in claim 40, wherein each sensor of the plurality of sensors has its own dedicated signal conditioning path.

62. The wearable biometric monitoring system as defined in claim 40, further comprising a multiplexer configured to selectively couple the signal conditioner to one of the plurality of sensors.

63. A method of fabricating a sweat analyte sensing system, the method comprising:
patterning a flexible substrate with a sweat analyte sensor array;
depositing a metal on the sweat analyte sensor array;
depositing an insulating layer on the sweat analyte sensor array;
defining electrode areas using photolithography and etching of the insulator;
patterning a metal on the electrode areas; and
forming reference electrodes corresponding to the electrode areas.

64. A wearable biometric monitoring system comprising:
a first sensor configured to sense a first analyte;
a second sensor configured to sense a second analyte;
a signal conditioner coupled to the first sensor and the second sensor, the signal conditioner configured receive and condition sensor signals from the first sensor and the second sensor, the signal conditioner comprising one or more amplifiers and one or more filters;
an interface configured to transmit information corresponding to the conditioned sensor signals to a remote computing device.
